SEQUENCE LISTING (25 sequences)

SEQ ID NO: 1 - DNA, 4956 bp, synthetic construct
  source 1..4956: mol_type = other DNA
atgcatagagacaatcctgtcttccacctctccccccggggaaaactccgttgcctgatcgccggcctgctgctggcgccccacctgccccaggctttcgccggcgggctggaggcggccggcgggccgggcggcacgccgcaactgctcaaccagggcggcgtgcccatcgtcaatatcgtcgcgccgaacgccgccggcctgtcgcacaaccagttcctcgactacaacgtcgaccgccagggcctggtcctcaacaatgcgctgcaagccggcgcctcgcagctcgccggacaactggcggccaacccgcagctgcgcggcgacgccgccagtgccatcctcaacgaagtgatcagccgcaacgcctcggcgatcaacggcccgcaggaaatcttcggccaggccgccgactacatcctggccaaccccaacggcatctcggtgaacggcggcagcttcatcaacacgccccatgccagcctgctggtcggccgtccggaactggccgacggcaagctgcaggcgctgaacaccaacgacgccgtcggcgccctgcagatccagggccagggcctgagcaaccgggatggcagcatcgccctgctggcgccgcgagtcgacagccaaggcaagatcgaggcgtcggccgagctcgacctgaccgtcggtcgcaaccgcatcgactacccgagcggcaaggtcgagcgcgacccgtccggcgacgtccgtccgggcgagcggcgcatcgacgccagcctgttcggcgccatgcaggccggacgcatcaacatcctcagcaccgccgagggcgccggcgtgcgcgtcggcccggtcgggatcgacggcagggacggcgtcgacctgcgctccgccggcgacctgtcgatcagcggccaggcgcttcccgacaacagcctgaacgccttgcgcgcggcgatccgcagcgacagcggcaacgtcggcctgcatgcccgcggcgacctgagcctggccgccgccgacgtcagcggcggcagggtcgacctcaagtccgggcgcaacctgaccctgggcagcgtggaaagccgcaacctgcgggaaagccgcgagcgctggagcaacagcaccatcggcatcacctgggaaacctacgaccgcacgcgtaccgtcaccgattcgaagcagcacggcagccggatcgatgcccgcgccgacgcgagcctggccgcgcggggcgacagcgaactgagggcggcgacggtcaaggccggcgctaccctgaaagtctccagcggcggcgatacccgtcttctcgccgccaccgagacccgcaccgaacgcgaccagggcgcgcaccgcaagcacctgtggaaagccaactgggacaagggcagcagcgagcaacgcagcgtggccagcagcctcgaaggcgcgcgggtcgaactcggcggcggccggcgcctgaatctggaaggcgccgacgtggccagccgcggcgacctcgacctgcaagccaagagcgtcgacatcggcagcgccagccgcagccacagcagccgcgacaacagctactccggcgacctggtcggcggcagcttcttcggcagccacggcgatggcgacagcggcaagaccctgcaacagggcagccgggtcaaggccgacggcgcactgacggtgaccgccgatgcggtcgaggtgcgcggcagccaggtacgcggcgcgcgcaaggccgaggtagtcagcgggaaaggctcgctgcgcatcgacggcgtggaggaaaccgcccacagcaacagctacagcaaggacagcaagttcttcggcatcgccaaggacgagagtcgccagcgcagcaaggacagcagcaaccgcgccagcgaagtacgctcggacagcaacctgaccctgcgcagcgccgccgacatcgccattcgcggctcccgggtcgaggctggcggcgccctcgccgccgaggccaagggcaacctggagatagcctcggcgcaggagcgccacgacggcaacgacagccgccacacccgcggcttcgacgcctatgccggcgagcagaccccaggcagccgccaataccgcgccggggtgcgctaccaggaccagcggaccagcgtccgccgcgaggaaacccgcaacagcggctccagcctgggcggcgcctccctggccgtgaaggccggcggcgatctcaccgtgaagggggccgagctgaaggccagcgcgggcgacgccagcctctccgggaagaacgtcgccctgctcgccgagcaggacagcaagacccgcagcagcgaacagaccaccaccggcggcggcttctactataccggcggcctggaccgtgccggcagcggtatcgaagtcggccaccagcggatcgacgagaacgacgccgaaagccatgcgcgcaccagccaggtgaacgcgacgggcaatctcaggatcgacgccgcccagggcagcctgacgacgcagggcgcgcgcctggaggccggcgacagcctggcggtcgccgccggcacggtcgacaaccaggccgcccgcgacagccagagcagccagcgccacgacagcggctggagcggcgacatcggcgccaacctcgagtaccgcggcatcgcccggccgatcgagaaggcggtcgaaggcgtcgcccagcgcaaggtccaccagcccggcctgctcgacaacctggagcagccgaacgtcggcgtcgacctcgagatcagccaccgcgacagccgcggcgagcaacaggcgagccaggcgcaggtcagcagcttcgcgggcggccaggtcgaactgaaggtcggcgacgccctgcgggacgagggcacccgttaccaggctcgcagcggaggcctcctcatcgacgccgccaggcatgacgccagggcggcggagaacacctccggcagccatgagcagagcctcgacgccaaggtcggcgggcgcctctacaccaccaccggccaggacctgaacctgcgcctgagcggcattggcggcagcagcgagaacagcgccagccagaccaccgcggtggtcggcgaatacgccgcgaagcagggcgtcgagatccgcctcggcggagacggcctctaccagggcagccgcttcgacggcggcgaagccggggtcaggctcagcgccggcggcaacctggccctggaacaggccaacgaccggcagagcgccagcagcgccagcctgcgtggcgacgccgcgttgagcggcggcatggcccccagcgccaacggcaaagggctgaacgccagcgccggcctgcaactcgaccacaaggccggcgacagccgggacagccaggcgcgggtcgccgacatccaggccaagggcgcggtggagctgcgcagcggcggcgatctggtcctgcaaggcagcaatatcggcagcgcggcagcgaagaccggcgacatagtcctggccgccggcggcaagctcgacctgcaggccgcccgcgatagccaccgggccgggggaaacaacctcggcggcggcttcagcctaggcggcggcagcgttcgcgacgccgaaacgagcagcaagaacggcagcgtcagcggcaacttcaacatcggccgggtcgacgaggaacgtcacgcgctgaacggcggcaacctgcacagcgcgaccaaggccagcctttccagcgccgcagacgacgccaccgcggtacgcctgcaaggcactcgcatcgaagccgcccaggtcagcctcgaagccggcaacggcggcattctccaggagtccgccgaatccagcgagcggcgcgacaactggggcgtgctgctcggtgccggggccaacggcggcaagaccaccggcgcgccgagcgactaccggagcgactatgccgtccaggcccgcgccaaggtcgatgtcgatgtcctgcgcagccagacccagggcgacagcgtcatccaggccgaccgggtaatcctggcgagccagggcgacacccgcctggagggcgcgcgcatcgacgcggcacaggtggacgggcgcatcggcggcgacctgcgggtggagagccgccaggaccgcgcggagggcgtgaaggtcaacgtcgacgcgcgcctgggcgtggagaagaaccagcccggcctggtgaacaagctggcgagcaagaccggaccgttgaaggacaagctggaaaccaaggccgagaatgctttcgacaagcaccgcggcaagttggagaacggcatcgaccgtaatgtcgagcggctcggcaaggccggggacaacctcctcgccaaagccgaaaaggccaaggagcgcctgggcgagaagctggtccgcagcggcagctacgaggtcaacccggagccgcgcggcgccttcgccagcaagctggacagggccaggggctatctggcggagaaaggcgaagcgctcggcgaccggctgtccggcctcaagcagcgcctgtcgccgaacaagaccggtagctatgtggtgaacgacaagcagacggccggcgccaaggtcggcaatgccgccgagaacgtgctgttcggcgacaagagcggcgaagcctcggtaaccccgacgctgtacctggacgtcagccacgtcagccgcaactacgtcaccgaggcctccggcatcaccggcaggcagggcgtgaacctgcaggtgggcgcagcgacccagctgaccggcgcacggatcagcgccagcgacggcaaggtcgacctcggcggctcgcgcgtggaaacccgcgccctggccggcaaggactaccgcgccgatctcggcctgaacgtctccaggtcgccggtggacctggccttcggtatcaaggacgagttcagccaggagcacgaccaggcgacccgcgacgaccaggccttcaacctcggcgccctgcgcgtcggcggacgcaaccgcgaccagcagttgcaggccggcatcgagcagaaggccgactga

SEQ ID NO: 2 - protein, 1651 aa, synthetic construct
MHRDNPVFHLSPRGKLRCLIAGLLLAPHLPQAFAGGLEAAGGPGGTPQLLNQGGVPIVNIVAPNAAGLSHNQFLDYNVDRQGLVLNNALQAGASQLAGQLAANPQLRGDAASAILNEVISRNASAINGPQEIFGQAADYILANPNGISVNGGSFINTPHASLLVGRPELADGKLQALNTNDAVGALQIQGQGLSNRDGSIALLAPRVDSQGKIEASAELDLTVGRNRIDYPSGKVERDPSGDVRPGERRIDASLFGAMQAGRINILSTAEGAGVRVGPVGIDGRDGVDLRSAGDLSISGQALPDNSLNALRAAIRSDSGNVGLHARGDLSLAAADVSGGRVDLKSGRNLTLGSVESRNLRESRERWSNSTIGITWETYDRTRTVTDSKQHGSRIDARADASLAARGDSELRAATVKAGATLKVSSGGDTRLLAATETRTERDQGAHRKHLWKANWDKGSSEQRSVASSLEGARVELGGGRRLNLEGADVASRGDLDLQAKSVDIGSASRSHSSRDNSYSGDLVGGSFFGSHGDGDSGKTLQQGSRVKADGALTVTADAVEVRGSQVRGARKAEVVSGKGSLRIDGVEETAHSNSYSKDSKFFGIAKDESRQRSKDSSNRASEVRSDSNLTLRSAADIAIRGSRVEAGGALAAEAKGNLEIASAQERHDGNDSRHTRGFDAYAGEQTPGSRQYRAGVRYQDQRTSVRREETRNSGSSLGGASLAVKAGGDLTVKGAELKASAGDASLSGKNVALLAEQDSKTRSSEQTTTGGGFYYTGGLDRAGSGIEVGHQRIDENDAESHARTSQVNATGNLRIDAAQGSLTTQGARLEAGDSLAVAAGTVDNQAARDSQSSQRHDSGWSGDIGANLEYRGIARPIEKAVEGVAQRKVHQPGLLDNLEQPNVGVDLEISHRDSRGEQQASQAQVSSFAGGQVELKVGDALRDEGTRYQARSGGLLIDAARHDARAAENTSGSHEQSLDAKVGGRLYTTTGQDLNLRLSGIGGSSENSASQTTAVVGEYAAKQGVEIRLGGDGLYQGSRFDGGEAGVRLSAGGNLALEQANDRQSASSASLRGDAALSGGMAPSANGKGLNASAGLQLDHKAGDSRDSQARVADIQAKGAVELRSGGDLVLQGSNIGSAAAKTGDIVLAAGGKLDLQAARDSHRAGGNNLGGGFSLGGGSVRDAETSSKNGSVSGNFNIGRVDEERHALNGGNLHSATKASLSSAADDATAVRLQGTRIEAAQVSLEAGNGGILQESAESSERRDNWGVLLGAGANGGKTTGAPSDYRSDYAVQARAKVDVDVLRSQTQGDSVIQADRVILASQGDTRLEGARIDAAQVDGRIGGDLRVESRQDRAEGVKVNVDARLGVEKNQPGLVNKLASKTGPLKDKLETKAENAFDKHRGKLENGIDRNVERLGKAGDNLLAKAEKAKERLGEKLVRSGSYEVNPEPRGAFASKLDRARGYLAEKGEALGDRLSGLKQRLSPNKTGSYVVNDKQTAGAKVGNAAENVLFGDKSGEASVTPTLYLDVSHVSRNYVTEASGITGRQGVNLQVGAATQLTGARISASDGKVDLGGSRVETRALAGKDYRADLGLNVSRSPVDLAFGIKDEFSQEHDQATRDDQAFNLGALRVGGRNRDQQLQAGIEQKAD

SEQ ID NO: 3 - DNA, 876 bp, synthetic construct
  source 1..876: mol_type = other DNA
gtcaacgtcgacgcgcgcctgggcgtggagaagaaccagcccggcctggtgaacaagctggcgagcaagaccggaccgttgaaggacaagctggaaaccaaggccgagaatgctttcgacaagcaccgcggcaagttggagaacggcatcgaccgtaatgtcgagcggctcggcaaggccggggacaacctcctcgccaaagccgaaaaggccaaggagcgcctgggcgagaagctggtccgcagcggcagctacgaggtcaacccggagccgcgcggcgccttcgccagcaagctggacagggccaggggctatctggcggagaaaggcgaagcgctcggcgaccggctgtccggcctcaagcagcgcctgtcgccgaacaagaccggtagctatgtggtgaacgacaagcagacggccggcgccaaggtcggcaatgccgccgagaacgtgctgttcggcgacaagagcggcgaagcctcggtaaccccgacgctgtacctggacgtcagccacgtcagccgcaactacgtcaccgaggcctccggcatcaccggcaggcagggcgtgaacctgcaggtgggcgcagcgacccagctgaccggcgcacggatcagcgccagcgacggcaaggtcgacctcggcggctcgcgcgtggaaacccgcgccctggccggcaaggactaccgcgccgatctcggcctgaacgtctccaggtcgccggtggacctggccttcggtatcaaggacgagttcagccaggagcacgaccaggcgacccgcgacgaccaggccttcaacctcggcgccctgcgcgtcggcggacgcaaccgcgaccagcagttgcaggccggcatcgagcagaaggccgactga

SEQ ID NO: 4 - protein, 291 aa, synthetic construct
VNVDARLGVEKNQPGLVNKLASKTGPLKDKLETKAENAFDKHRGKLENGIDRNVERLGKAGDNLLAKAEKAKERLGEKLVRSGSYEVNPEPRGAFASKLDRARGYLAEKGEALGDRLSGLKQRLSPNKTGSYVVNDKQTAGAKVGNAAENVLFGDKSGEASVTPTLYLDVSHVSRNYVTEASGITGRQGVNLQVGAATQLTGARISASDGKVDLGGSRVETRALAGKDYRADLGLNVSRSPVDLAFGIKDEFSQEHDQATRDDQAFNLGALRVGGRNRDQQLQAGIEQKAD

SEQ ID NO: 5 - DNA, 2070 bp, synthetic construct
  source 1..2070: mol_type = other DNA
catatggaagtcagaaaccttaatgccgctcgcgagctgttcctggacgagctcctggccgcgtcggcggcgcctgccagtgccgagcaggaggaactgctggccctgttgcgcagcgagcggatcgtgctggcccacgccggccagccgctgagcgaggcgcaagtgctcaaggcgctcgcctggttgctcgcggccaatccgtccgcgcctccggggcagggcctcgaggtactccgcgaagtcctgcaggcacgtcggcagcccggtgcgcagtgggatctgcgtgagttcctggtgtcggcctatttcagcctgcacgggcgtctcgacgaggatgtcatcggtgtctacaaggatgtcctgcagacccaggacggcaagcgcaaggcgctgctcgacgagctcaaggcgctgaccgcggagttgaaggtctacagcgtgatccagtcgcagatcaacgccgcgctgtcggccaggcagggcatcaggatcgacgctggcggtatcgatctggtcgaccccacgctatatggctatgccgtcggcgatcccaggtggaaggacagccccgagtatgcgctgctgagcaatctggataccttcagcggcaagctgtcgatcaaggattttctcagcggctcgccgaagcagagcggggaactcaagggcctcagcgatgagtaccccttcgagaaggacaacaacccggtcggcaatttcgccaccacggtgagcgaccgctcgcgtccgctgaacgacaaggtcaacgagaagaccaccctgctcaacgacaccagctcccgctacaactcggcggtcgaggcgctcaaccgcttcatccagaaatacgacagcgtcctgagcgacattctcagcgcgatcggatccatgaacccgattacgctggaacgtgctggtctgccgtatggtgttgccgatgctggtgacatcccggctctgggtcgcccggtcgcacgtgatgtggaaagtctgcgtgttgaacgtctggcagcaccggcagctgcaagcgcatctggcaccggtgtcgctctgacgccgccgtctgcagcaagtcagcaacgtctggaagttgctaaccgcgcggaaattgcctcactggtccaggcagtgggtgaagacgtgggtctggcacgtcaagtggttctggcaggtgcatcgaccctgctgagcgcaggtctgatgtcgccgcaggcgttcgaaattgaactggccaaaatcaccggcgaagttgaaaatcagcagaaaaaactgaaactgacggaaatcgaacaggcccgtaaacagaacctgcaaaaaatggaagataaccagcaaaaaatccgcgaatcggaagaagctgcgaaagaagcgcagaaaagcggcctggccgcaaaaatttttggttggatttctgctatcgcgagtattatcgtgggtgcaatcatggttgcaaccggtgtcggtgctgcagcaggtgcactgatgattgctggcggtgtcatgggtgtcgtgagtcagtccgtgcagcaagcagctgcggatggtctgatctcaaaagaagtgatggaaaaactgggcccggccctgatgggtattgaaatggccgtggcactgctggccgcagttgtctcctttggtggttcagcagttggtggtctggcacgtctgggtgcaaaaatcggcggtaaagctgcggaaatgacggcatccctggcttcaaaagtggcagacctgggcggtaaattcggctctctggcgggccagtcactgtcgcatagcctgaaactgggtgtgcaagtttctgatctgaccctggacgttgcaaacggcgccgcacaggctacgcacagtggttttcaagcgaaagctgcgaatcgtcaggccgatgttcaagaatcccgtgcagacctgaccacgctgcagggtgtcattgaacgtctgaaagaagaactgagccgcatgctggaagcctttcaggaaattatggaacgcatcttcgcaatgctgcaagcgaaaggcgaaaccctgcacaatctgtcttcccgtccggcggctatctgaggatcc

SEQ ID NO: 6 - protein, 686 aa, synthetic construct
MEVRNLNAARELFLDELLAASAAPASAEQEELLALLRSERIVLAHAGQPLSEAQVLKALAWLLAANPSAPPGQGLEVLREVLQARRQPGAQWDLREFLVSAYFSLHGRLDEDVIGVYKDVLQTQDGKRKALLDELKALTAELKVYSVIQSQINAALSARQGIRIDAGGIDLVDPTLYGYAVGDPRWKDSPEYALLSNLDTFSGKLSIKDFLSGSPKQSGELKGLSDEYPFEKDNNPVGNFATTVSDRSRPLNDKVNEKTTLLNDTSSRYNSAVEALNRFIQKYDSVLSDILSAIGSMNPITLERAGLPYGVADAGDIPALGRPVARDVESLRVERLAAPAAASASGTGVALTPPSAASQQRLEVANRAEIASLVQAVGEDVGLARQVVLAGASTLLSAGLMSPQAFEIELAKITGEVENQQKKLKLTEIEQARKQNLQKMEDNQQKIRESEEAAKEAQKSGLAAKIFGWISAIASIIVGAIMVATGVGAAAGALMIAGGVMGVVSQSVQQAAADGLISKEVMEKLGPALMGIEMAVALLAAVVSFGGSAVGGLARLGAKIGGKAAEMTASLASKVADLGGKFGSLAGQSLSHSLKLGVQVSDLTLDVANGAAQATHSGFQAKAANRQADVQESRADLTTLQGVIERLKEELSRMLEAFQEIMERIFAMLQAKGETLHNLSSRPAAI

SEQ ID NO: 7 - DNA, 2655 bp, synthetic construct
  source 1..2655: mol_type = other DNA
catatggacaatggcgatcgtttataccgtgccgactcgcgtcccccagatgagattaaacgtagcggtgggttaatgccacgtgggcacaatgagtattttgaccgtggaacacagatgaacattaacctttacgatcatgcccgtgggacccagaccgggtttgtccgttatgatgacgggtatgttagtacgagtttgtccttacgctccgcacaccttgcgggacaaagtattttatcaggctacagcacatattacatttatgtgatcgccactgccccaaacatgttcaatgtgaacgatgtgttgggggtttacagcccccatccatatgaacaagaagtctcggcccttggggggatcccatatagccagatttatggttggtaccgcgtaaattttggtgtgattgatgaacgtttgcatcgtaaccgtgaataccgcgatcgctactaccgtaacttgaacattgcacctgccgaggacggctatcgtttagcgggattcccacccgatcatcaggcgtggcgtgaggaaccgtggatccatcacgcccctcaggggtgcgggaacagtagtcgccatatggaagtcagaaaccttaatgccgctcgcgagctgttcctggacgagctcctggccgcgtcggcggcgcctgccagtgccgagcaggaggaactgctggccctgttgcgcagcgagcggatcgtgctggcccacgccggccagccgctgagcgaggcgcaagtgctcaaggcgctcgcctggttgctcgcggccaatccgtccgcgcctccggggcagggcctcgaggtactccgcgaagtcctgcaggcacgtcggcagcccggtgcgcagtgggatctgcgtgagttcctggtgtcggcctatttcagcctgcacgggcgtctcgacgaggatgtcatcggtgtctacaaggatgtcctgcagacccaggacggcaagcgcaaggcgctgctcgacgagctcaaggcgctgaccgcggagttgaaggtctacagcgtgatccagtcgcagatcaacgccgcgctgtcggccaggcagggcatcaggatcgacgctggcggtatcgatctggtcgaccccacgctatatggctatgccgtcggcgatcccaggtggaaggacagccccgagtatgcgctgctgagcaatctggataccttcagcggcaagctgtcgatcaaggattttctcagcggctcgccgaagcagagcggggaactcaagggcctcagcgatgagtaccccttcgagaaggacaacaacccggtcggcaatttcgccaccacggtgagcgaccgctcgcgtccgctgaacgacaaggtcaacgagaagaccaccctgctcaacgacaccagctcccgctacaactcggcggtcgaggcgctcaaccgcttcatccagaaatacgacagcgtcctgagcgacattctcagcgcgatcggatccatgaacccgattacgctggaacgtgctggtctgccgtatggtgttgccgatgctggtgacatcccggctctgggtcgcccggtcgcacgtgatgtggaaagtctgcgtgttgaacgtctggcagcaccggcagctgcaagcgcatctggcaccggtgtcgctctgacgccgccgtctgcagcaagtcagcaacgtctggaagttgctaaccgcgcggaaattgcctcactggtccaggcagtgggtgaagacgtgggtctggcacgtcaagtggttctggcaggtgcatcgaccctgctgagcgcaggtctgatgtcgccgcaggcgttcgaaattgaactggccaaaatcaccggcgaagttgaaaatcagcagaaaaaactgaaactgacggaaatcgaacaggcccgtaaacagaacctgcaaaaaatggaagataaccagcaaaaaatccgcgaatcggaagaagctgcgaaagaagcgcagaaaagcggcctggccgcaaaaatttttggttggatttctgctatcgcgagtattatcgtgggtgcaatcatggttgcaaccggtgtcggtgctgcagcaggtgcactgatgattgctggcggtgtcatgggtgtcgtgagtcagtccgtgcagcaagcagctgcggatggtctgatctcaaaagaagtgatggaaaaactgggcccggccctgatgggtattgaaatggccgtggcactgctggccgcagttgtctcctttggtggttcagcagttggtggtctggcacgtctgggtgcaaaaatcggcggtaaagctgcggaaatgacggcatccctggcttcaaaagtggcagacctgggcggtaaattcggctctctggcgggccagtcactgtcgcatagcctgaaactgggtgtgcaagtttctgatctgaccctggacgttgcaaacggcgccgcacaggctacgcacagtggttttcaagcgaaagctgcgaatcgtcaggccgatgttcaagaatcccgtgcagacctgaccacgctgcagggtgtcattgaacgtctgaaagaagaactgagccgcatgctggaagcctttcaggaaattatggaacgcatcttcgcaatgctgcaagcgaaaggcgaaaccctgcacaatctgtcttcccgtccggcggctatctgaggatcc

SEQ ID NO: 8 - protein, 880 aa, synthetic construct
MDNGDRLYRADSRPPDEIKRSGGLMPRGHNEYFDRGTQMNINLYDHARGTQTGFVRYDDGYVSTSLSLRSAHLAGQSILSGYSTYYIYVIATAPNMFNVNDVLGVYSPHPYEQEVSALGGIPYSQIYGWYRVNFGVIDERLHRNREYRDRYYRNLNIAPAEDGYRLAGFPPDHQAWREEPWIHHAPQGCGNSSRMEVRNLNAARELFLDELLAASAAPASAEQEELLALLRSERIVLAHAGQPLSEAQVLKALAWLLAANPSAPPGQGLEVLREVLQARRQPGAQWDLREFLVSAYFSLHGRLDEDVIGVYKDVLQTQDGKRKALLDELKALTAELKVYSVIQSQINAALSARQGIRIDAGGIDLVDPTLYGYAVGDPRWKDSPEYALLSNLDTFSGKLSIKDFLSGSPKQSGELKGLSDEYPFEKDNNPVGNFATTVSDRSRPLNDKVNEKTTLLNDTSSRYNSAVEALNRFIQKYDSVLSDILSAIGSMNPITLERAGLPYGVADAGDIPALGRPVARDVESLRVERLAAPAAASASGTGVALTPPSAASQQRLEVANRAEIASLVQAVGEDVGLARQVVLAGASTLLSAGLMSPQAFEIELAKITGEVENQQKKLKLTEIEQARKQNLQKMEDNQQKIRESEEAAKEAQKSGLAAKIFGWISAIASIIVGAIMVATGVGAAAGALMIAGGVMGVVSQSVQQAAADGLISKEVMEKLGPALMGIEMAVALLAAVVSFGGSAVGGLARLGAKIGGKAAEMTASLASKVADLGGKFGSLAGQSLSHSLKLGVQVSDLTLDVANGAAQATHSGFQAKAANRQADVQESRADLTTLQGVIERLKEELSRMLEAFQEIMERIFAMLQAKGETLHNLSSRPAAI

SEQ ID NO: 9 - DNA, 588 bp, synthetic construct
  source 1..588: mol_type = other DNA
atgtcttcaggaaacatcttatggggaagtcaaaaccctattgtgtttaaaaatagcttcggcgtcagcaacgctgataccgggagccaggatgacttatcccagcaaaatccgtttgccgaagggtatggtgttttgcttattctccttatggttattcaggctatcgcaaataataaatttattgaagtccagaagaacgctgaacgtgccagaaatacccaggaaaagtcaaatgagatggatgaggtgattgctaaagcagccaaaggggatgctaaaaccaaagaggaggtgcctgaggatgtaattaaatacatgcgtgataatggtattctcatcgatggtatgaccattgatgattatatggctaaatatggcgatcatgggaagctggataaaggtggcctacaggcgatcaaagcggctttggataatgacgccaaccggaataccgatcttatgagtcaggggcagataacaattcaaaaaatgtctcaggagcttaacgctgtccttacccaactgacagggcttatcagtaagtggggggaaatttccagtatgatagcgcagaaaacgtactca

SEQ ID NO: 10 - protein, 196 aa, synthetic construct
MSSGNILWGSQNPIVFKNSFGVSNADTGSQDDLSQQNPFAEGYGVLLILLMVIQAIANNKFIEVQKNAERARNTQEKSNEMDEVIAKAAKGDAKTKEEVPEDVIKYMRDNGILIDGMTIDDYMAKYGDHGKLDKGGLQAIKAALDNDANRNTDLMSQGQITIQKMSQELNAVLTQLTGLISKWGEISSMIAQKTYS

SEQ ID NO: 11 - DNA, 1254 bp, synthetic construct
  source 1..1254: mol_type = other DNA
atgggcagcagccatcatcatcatcatcacagcagcggcctggtgccgcgcggcagccatatggacaatggcgatcgtttataccgtgccgactcgcgtcccccagatgagattaaacgtagcggtgggttaatgccacgtgggcacaatgagtattttgaccgtggaacacagatgaacattaacctttacgatcatgcccgtgggacccagaccgggtttgtccgttatgatgacgggtatgttagtacgagtttgtccttacgctccgcacaccttgcgggacaaagtattttatcaggctacagcacatattacatttatgtgatcgccactgccccaaacatgttcaatgtgaacgatgtgttgggggtttacagcccccatccatatgaacaagaagtctcggcccttggggggatcccatatagccagatttatggttggtaccgcgtaaattttggtgtgattgatgaacgtttgcatcgtaaccgtgaataccgcgatcgctactaccgtaacttgaacattgcacctgccgaggacggctatcgtttagcgggattcccacccgatcatcaggcgtggcgtgaggaaccgtggatccatcacgcccctcaggggtgcgggaacagtagtcgcgggtccgcggcatccatgtcttcaggaaacatcttatggggaagtcaaaaccctattgtgtttaaaaatagcttcggcgtcagcaacgctgataccgggagccaggatgacttatcccagcaaaatccgtttgccgaagggtatggtgttttgcttattctccttatggttattcaggctatcgcaaataataaatttattgaagtccagaagaacgctgaacgtgccagaaatacccaggaaaagtcaaatgagatggatgaggtgattgctaaagcagccaaaggggatgctaaaaccaaagaggaggtgcctgaggatgtaattaaatacatgcgtgataatggtattctcatcgatggtatgaccattgatgattatatggctaaatatggcgatcatgggaagctggataaaggtggcctacaggcgatcaaagcggctttggataatgacgccaaccggaataccgatcttatgagtcaggggcagataacaattcaaaaaatgtctcaggagcttaacgctgtccttacccaactgacagggcttatcagtaagtggggggaaatttccagtatgatagcgcagaaaacgtactcataaggatcc

SEQ ID NO: 12 - protein, 415 aa, synthetic construct
MGSSHHHHHHSSGLVPRGSHMDNGDRLYRADSRPPDEIKRSGGLMPRGHNEYFDRGTQMNINLYDHARGTQTGFVRYDDGYVSTSLSLRSAHLAGQSILSGYSTYYIYVIATAPNMFNVNDVLGVYSPHPYEQEVSALGGIPYSQIYGWYRVNFGVIDERLHRNREYRDRYYRNLNIAPAEDGYRLAGFPPDHQAWREEPWIHHAPQGCGNSSRGSAASMSSGNILWGSQNPIVFKNSFGVSNADTGSQDDLSQQNPFAEGYGVLLILLMVIQAIANNKFIEVQKNAERARNTQEKSNEMDEVIAKAAKGDAKTKEEVPEDVIKYMRDNGILIDGMTIDDYMAKYGDHGKLDKGGLQAIKAALDNDANRNTDLMSQGQITIQKMSQELNAVLTQLTGLISKWGEISSMIAQKTYS

SEQ ID NO: 13 - DNA, 585 bp, synthetic construct
  source 1..585: mol_type = other DNA
catatggacaatggcgatcgtttataccgtgccgactcgcgtcccccagatgagattaaacgtagcggtgggttaatgccacgtgggcacaatgagtattttgaccgtggaacacagatgaacattaacctttacgatcatgcccgtgggacccagaccgggtttgtccgttatgatgacgggtatgttagtacgagtttgtccttacgctccgcacaccttgcgggacaaagtattttatcaggctacagcacatattacatttatgtgatcgccactgccccaaacatgttcaatgtgaacgatgtgttgggggtttacagcccccatccatatgaacaagaagtctcggcccttggggggatcccatatagccagatttatggttggtaccgcgtaaattttggtgtgattgatgaacgtttgcatcgtaaccgtgaataccgcgatcgctactaccgtaacttgaacattgcacctgccgaggacggctatcgtttagcgggattcccacccgatcatcaggcgtggcgtgaggaaccgtggatccatcacgcccctcaggggtgcgggaacagtagtcgc

SEQ ID NO: 14 - protein, 585 aa, synthetic construct
CATATGGACAATGGCGATCGTTTATACCGTGCCGACTCGCGTCCCCCAGATGAGATTAAACGTAGCGGTGGGTTAATGCCACGTGGGCACAATGAGTATTTTGACCGTGGAACACAGATGAACATTAACCTTTACGATCATGCCCGTGGGACCCAGACCGGGTTTGTCCGTTATGATGACGGGTATGTTAGTACGAGTTTGTCCTTACGCTCCGCACACCTTGCGGGACAAAGTATTTTATCAGGCTACAGCACATATTACATTTATGTGATCGCCACTGCCCCAAACATGTTCAATGTGAACGATGTGTTGGGGGTTTACAGCCCCCATCCATATGAACAAGAAGTCTCGGCCCTTGGGGGGATCCCATATAGCCAGATTTATGGTTGGTACCGCGTAAATTTTGGTGTGATTGATGAACGTTTGCATCGTAACCGTGAATACCGCGATCGCTACTACCGTAACTTGAACATTGCACCTGCCGAGGACGGCTATCGTTTAGCGGGATTCCCACCCGATCATCAGGCGTGGCGTGAGGAACCGTGGATCCATCACGCCCCTCAGGGGTGCGGGAACAGTAGTCGC

SEQ ID NO: 15 - protein, 607 aa, synthetic construct
MTIDLGVSLTSQAGGLQGIDLKSMDIQTLMVYVQGRRAELLTAQMQTQAEVVQKANERMAQLNEVLSALSRAKAEFPPNPKPGDTIPGWDNQKVSRIEVPLNDALRAAGLTGMFEARDGQVTAPGGRGTQVVNGTGVMAGSTTYKELESAYTTVKGMLDTASNTQQMDMIRLQAASNKRNEAFEVMTNTEKRRSDLNSSITNNMRKLMTVMSTTISTAPSGAALAPSRIDMRAPEPGSAGEGAGILAPVTTLALAAGRPAFPASPSLRTAPVLDPPVRDLSPADLADLLRVLRSRAVDGQLATARENLQDAQVKAKQNTQAQLDKLDAWFRKAEEAESKGWLSKVFGWIGKVLAVVASALAVGFAAVASVATGAAATPMLLLSGMALVSAVTSLADQISQEAGGPPISLGGFLSGLAGRLLTALGVDQSQADQIAKIVAGLAVPVVLLIEPQMLGEMAQGVARLAGASDATAGYIAMAMSIVAAIAVAAINAAGTAGAGSASAIKGAWDRAAAVATQVLQGGTAVAQGGVGVSMAVDRKQADLLVADKADLAASLTKLRAAMEREADDIKKILAQFDEAYHMIAKMISDMASTHSQVSANLGRRQAV

SEQ ID NO: 16 - DNA, 618 bp, synthetic construct
  source 1..618: mol_type = other DNA
catatgaccattgatctcggagtttcactcacgtcgcaggccggcggcctgcaaggcatcgacctcaagagcatggatatccagactctcatggtgtatgtgcagggtcgtcgcgccgaactcctcacggctcaaatgcagacccaggccgaagtggtgcagaaggccaatgaacgcatggcgcagctcaacgaggtcctgtccgcgctgtcccgggccaaggccgagtttccgcccaatccgaagccgggcgacaccatcccgggctgggacaaccagaaggtcagccggatcgaggttcctctcaatgatgcgctgcgcgctgccggcctgacgggcatgttcgaagcgcgcgatggccaagtgaccgcccccggcggccggggtacgcaggtcgtgaacggcacgggcgtcatggccggttccacgacctataaggaactcgaaagtgcctacaccaccgtaaaggggatgctggatacggcgtccaatacgcaacagatggacatgatcaggctgcaggccgccagcaacaagcgcaacgaggctttcgaggtcatgaccaacaccgagaagcggcgcagcgacctgaacagttccatcaccaacaacatgcgc

SEQ ID NO: 17 - protein, 205 aa, synthetic construct
MTIDLGVSLTSQAGGLQGIDLKSMDIQTLMVYVQGRRAELLTAQMQTQAEVVQKANERMAQLNEVLSALSRAKAEFPPNPKPGDTIPGWDNQKVSRIEVPLNDALRAAGLTGMFEARDGQVTAPGGRGTQVVNGTGVMAGSTTYKELESAYTTVKGMLDTASNTQQMDMIRLQAASNKRNEAFEVMTNTEKRRSDLNSSITNNMR

SEQ ID NO: 18 - DNA, 1209 bp, synthetic construct
  source 1..1209: mol_type = other DNA
atgaccgtcatgagtacgaccatatccacagccccgagcggcgccgcgcttgcgccgtctcgcatagatatgcgggcaccggagcccgggagtgccggcgaaggcgccggcatcctggcgccggtgacgacgctggctctggcggcgggccggccggcttttccagcgtcaccgtcgctgcgcaccgcgcccgtcctggatccgccagtgcgcgatctcagccccgccgacttggccgacctgctgcgcgtcttgcgatccagggcggtggacgggcagttggccacggcgcgcgagaacctgcaggacgcgcaagtcaaggcgaagcagaacacccaggcccagctcgacaagctggacgcatggtttcggaaggccgaagaggccgagagcaagggatggctgagcaaggtgttcggctggatcggcaaggtgctggcggtcgtggcatcggccctggcggtgggctttgccgccgtcgccagcgtggccaccggcgcggcggccacacccatgctgctgctcagcggcatggcactggtcagcgccgtgacatcgctggccgaccagatatcgcaagaggcgggaggcccgcctatcagcctgggcgggtttctctccgggctggccggacgtctgctgacagcgttgggggtggatcagtcgcaggccgaccaaattgccaagatcgtcgccggcctggccgtgcccgtcgtcttgctgatcgaaccccagatgctgggcgaaatggcgcaaggcgtggccaggctggctggcgccagcgatgccaccgcggggtacatagccatggcgatgtccatcgtggcggcgatcgcggtcgccgcgatcaatgccgccggtacagccggcgcgggtagcgcttcggcgatcaagggggcctgggatcgggccgccgcggtagccacccaggtccttcaagggggtacggcagtggcgcaaggcggcgtcggcgtgtcgatggcagtcgatcgcaaacaggccgatctcctggtcgccgacaaggcggatctggcggcgagcctgacaaaactgcgggcggccatggagcgtgaggcggacgatatcaagaagatcctggctcaattcgacgaggcctatcacatgatcgcgaagatgatcagcgatatggcgagtacgcacagccaggtcagcgccaacctcgggcggcgccaggcggtgtagctcgag

SEQ ID NO: 19 - protein, 400 aa, synthetic construct
MTVMSTTISTAPSGAALAPSRIDMRAPEPGSAGEGAGILAPVTTLALAAGRPAFPASPSLRTAPVLDPPVRDLSPADLADLLRVLRSRAVDGQLATARENLQDAQVKAKQNTQAQLDKLDAWFRKAEEAESKGWLSKVFGWIGKVLAVVASALAVGFAAVASVATGAAATPMLLLSGMALVSAVTSLADQISQEAGGPPISLGGFLSGLAGRLLTALGVDQSQADQIAKIVAGLAVPVVLLIEPQMLGEMAQGVARLAGASDATAGYIAMAMSIVAAIAVAAINAAGTAGAGSASAIKGAWDRAAAVATQVLQGGTAVAQGGVGVSMAVDRKQADLLVADKADLAASLTKLRAAMEREADDIKKILAQFDEAYHMIAKMISDMASTHSQVSANLGRRQAV

SEQ ID NO: 20 - protein, 1081 aa, synthetic construct
MDNGDRLYRADSRPPDEIKRSGGLMPRGHNEYFDRGTQMNINLYDHARGTQTGFVRYDDGYVSTSLSLRSAHLAGQSILSGYSTYYIYVIATAPNMFNVNDVLGVYSPHPYEQEVSALGGIPYSQIYGWYRVNFGVIDERLHRNREYRDRYYRNLNIAPAEDGYRLAGFPPDHQAWREEPWIHHAPQGCGNSSRGSAASMNITTLTNSISTSSFSPNNTNGSSTETVNSDIKTTTSSHPSSLTMLNDTLHNIRTTNQALKKELSQKTLRNEYPINKDARELLHSAPKEAELDGDQMISHRELWAKIANSINDINEQYLKVYEHAVSSYTQMYQDFSAVLSSLAGWISPGGNDGNSVKLQVNSLKKALEELKEKYKDKPLYPANNTVSQEQANKWLTELGGTIGKVSQKNGGYVVSINMTPIDNMLKSLDNLGGNGEVVLDNAKYQAWNGFSAEDETMKNNLQTLVQKYSNANSIFDNLVKVLSSTISSCTDTDKLFLHFLEMHNVSTTTTGFPLAKILTSTELGDNTIQAANDAANKLFSLTIADLTANQNINTTNAHSTSNILIPELKAPKSLNASSQLTLLIGNLIQILGEKSLTALTNKITAWKSQQQARQQKNLEFSDKINTLLSETEGLTRDYEKQINKLKNADSKIKDLENKINQIQTRLSNLDPESPEKKKLSREEIQLTIKKDAAVKDRTLIEQKTLSIHSKLTDKSMQLEKEIDSFSAFSNTASAEQLSTQQKSLTGLASVTQLMATFIQLVGKNNEESLKNDLALFQSLQESRKTEMERKSDEYAAEVRKAEELNRVMGCVGKILGALLTIVSVVAAAFSGGASLALAAVGLALMVTDAIVQAATGNSFMEQALNPIMKAVIEPLIKLLSDAFTKMLEGLGVDSKKAKMIGSILGAIAGALVLVAAVVLVATVGKQAAAKLAENIGKIIGKTLTDLIPKFLKNFSSQLDDLITNAVARLNKFLGAAGDEVISKQIISTHLNQAVLLGESVNSATQAGGSVASAVFQNSASTNLADLTLSKYQVEQLSKYISEAIEKFGQLQEVIADLLASMSNSQANRTDVAKAILQQTTA

SEQ ID NO: 21 - DNA, 46 bp, synthetic construct
  source 1..46: mol_type = other DNA
ctagcatgactggtggacagcaaatgggtcgcggatccggctgcta

SEQ ID NO: 22 - DNA, 777 bp, synthetic construct
  source 1..777: mol_type = other DNA
atggtaaagataatatttgtgttttttattttcttatcatcattttcatatgcaaatgatgataagttatatcgggcagattctagacctcctgatgaaataaagcagtcaggtggtcttatgccaagaggacagagtgagtactttgaccgaggtactcaaatgaatatcaacctttatgatcatgcaagaggaactcagacgggatttgttaggcacgatgatggatatgtttccacctcaattagtttgagaagtgcccacttagtgggtcaaactatattgtctggtcattctacttattatatatatgttatagccactgcacccaacatgtttaacgttaatgatgtattaggggcatacagtcctcatccagatgaacaagaagtttctgctttaggtgggattccatactcccaaatatatggatggtatcgagttcattttggggtgcttgatgaacaattacatcgtaataggggctacagagatagatattacagtaacttagatattgctccagcagcagatggttatggattggcaggtttccctccggagcatagagcttggagggaagagccgtggattcatcatgcaccgccgggttgtgggaatgctccaagatcatcgatcagtaatacttgcgatgaaaaaacccaaagtctaggtgtaaaattccttgacgaataccaatctaaagttaaaagacaaatattttcaggctatcaatctgatattgatacacataatagaattaaggatgaattatga

SEQ ID NO: 23 - protein, 258 aa, synthetic construct
MVKIIFVFFIFLSSFSYANDDKLYRADSRPPDEIKQSGGLMPRGQSEYFDRGTQMNINLYDHARGTQTGFVRHDDGYVSTSISLRSAHLVGQTILSGHSTYYIYVIATAPNMFNVNDVLGAYSPHPDEQEVSALGGIPYSQIYGWYRVHFGVLDEQLHRNRGYRDRYYSNLDIAPAADGYGLAGFPPEHRAWREEPWIHHAPPGCGNAPRSSMSNTCDEKTQSLGVKFLDEYQSKVKRQIFSGYQSDIDTHNRIKDEL

SEQ ID NO: 24 - DNA, 375 bp, synthetic construct
  source 1..375: mol_type = other DNA
atgattaaattaaaatttggtgttttttttacagttttactatcttcagcatatgcacatggaacacctcaaaatattactgatttgtgtgcagaataccacaacacacaaatatatacgctaaatgataagatattttcgtatacagaatctctagctggaaaaagagagatggctatcattacttttaagaatggtgcaatttttcaagtagaagtaccaggtagtcaacatatagattcacaaaaaaaagcgattgaaaggatgaaggataccctgaggattgcatatcttactgaagctaaagtcgaaaagttatgtgtatggaataataaaacgcctcatgcgattgccgcaattagtatggcaaattaa

SEQ ID NO: 25 - protein, 124 aa, synthetic construct
  REGION 1..124: note = Synthetic Construct
MIKLKFGVFFTVLLSSAYAHGTPQNITDLCAEYHNTQIYTLNDKIFSYTESLAGKREMAIITFKNGAIFQVEVPGSQHIDSQKKAIERMKDTLRIAYLTEAKVEKLCVWNNKTPHAIAAISMAN

(19) United States
(12) Patent Application Publication
(10) Pub. No.: US 2026/0284172 A1
(43) Pub. Date: Sep. 24, 2026

(54) A BROADLY PROTECTIVE PROPHYLACTIC VACCINE AGAINST PSEUDOMONAS AERUGINOSA

(71) Applicants: UNIVERSITY OF KANSAS, Lawrence, KS (US); UNIVERSITY OF MARYLAND, BALTIMORE, Baltimore, MD (US)

(72) Inventors: Wendy L. PICKING, Lawrence, KS (US); William D. PICKING, Lawrence, KS (US); Robert K. ERNST, Silver Spring, MD (US)

(21) Appl. No.: 19/377,934

(22) Filed: Nov. 3, 2025

Related U.S. Application Data
(63) Continuation of application No. 18/562,170, filed on May 23, 2022.
(60) Provisional application No. 63/191,688, filed on May 21, 2021.
(60) Provisional application No. 63/286,268, filed on Dec. 6, 2021.

(51) Int. Cl. A61K 39/104; A61K 39/00; A61K 39/02; A61K 39/112; A61P 37/04; C07K 14/195; C07K 14/21; C07K 14/235; C07K 14/24; C07K 14/245; C07K 14/25; C07K 14/255; C07K 14/28

(52) U.S. Cl. CPC A61K 39/104; A61K 39/0208; A61K 39/0275; A61K 39/0283; A61K 39/0291; A61K 39/099; A61P 37/04; C07K 14/195; C07K 14/21; C07K 14/235; C07K 14/24; C07K 14/245; C07K 14/25; C07K 14/255; C07K 14/28; A61K 2039/55566; A61K 2039/55572; A61K 2039/55583

(57) ABSTRACT

Disclosed are compositions comprising a fusion polypeptide comprising i) a fusion of a needle tip protein or an antigenic fragment thereof and/or a translocator protein or an antigenic fragment thereof from a Type III secretion system (T3SS) of a Gram negative bacteria and ii) the A1 subunit of the labile toxin (LTA1) from enterotoxigenic Escherichia coli or cholera toxin. and methods of their use.

This application is a divisional of U.S. application Ser. No. 18/562,170, filed on Nov. 17, 2023, which is a 35 USC 371 National Stage filing of PCT Application No. PCT/US2022/030565, filed on May 23, 2022, which claims the benefit of U.S. Provisional Application No. 63/286,268, filed on Dec. 6, 2021 and U.S. Provisional Application No. 63/191,688, filed on May 21, 2021, Applications which are incorporated herein by reference in their entireties.

This invention was made with government support under Grant No. R01AI138970 and R21AI140701 awarded by the National Institutes of Health. The government has certain rights in the invention.

### REFERENCE TO SEQUENCE LISTING

A Sequence Listing conforming to the rules of WIPO Standard ST.26 is hereby incorporated by reference. Said Sequence Listing has been filed as an electronic document via PatentCenter in ASCII format encoded as XML. The electronic document, created on Nov. 3, 2025, is entitled “10776-022US2_ST26.xml”, and is 46,362 bytes in size.

### I. BACKGROUND

Pseudomonas aeruginosa (Pa) is an important opportunistic human pathogen responsible for severe infections in patients with burns, severe wounds, pneumonia, and critically ill patients who require intubation (ventilator-associated pneumonia) or catheterization (urinary tract infections). Clearing Pa has become increasingly difficult due to innate and acquired antibiotic resistance. Multidrug-resistant (MDR) Pa was classified as a serious threat in the CDC Antibiotic Resistance Threats report 2019. In 2017, there were ~32,600 cases of MDR Pa infection in hospitalized patients causing an estimated 2700 deaths and costing $757 million in health care costs in the US. A 2016 report describes Pa as the most common Gram-negative infection among troops with combat-related injuries in Afghanistan with 10% being MDR. Pa is also the major cause of pulmonary infections in cystic fibrosis (CF) patients with >70% of this group being chronically colonized by their late teens. Pa infections in chronic pulmonary conditions such as chronic obstructive pulmonary disease (COPD) and non-CF bronchiectasis (nCFB) have poor prognoses. Despite this ability to cause disease in humans who have been injured or hospitalized, it is aging that represents the biggest risk factor for acquiring acute lethal Pa infection. Taken together, a better vaccine is needed.

### II. SUMMARY

Disclosed are methods and compositions related to polypeptides comprising a fusion of the needle tip protein and translocator protein of a type III secretion apparatus (T3SA) from a type III secretion system (T3SS) of a Gram negative bacteria.

Disclosed herein are fusion polypeptides comprising i) a fusion of a needle tip protein (such as, for example, PcrV, IpaD, SseB, Bsp22, LcrV, or BipD) or an antigenic fragment thereof and/or a translocator protein (such as, for example PopB, IpaB, SseC, BopB, YopB, or BipB) or an antigenic fragment thereof from a Type III secretion system (T3SS) of a Gram negative bacteria (such as, for example, Pseudomonas spp. (including, but not limited to Pseudomonas aeruginosa), Shigella spp, Salmonella enterica, Bordetella spp., Yersinia spp., or Burkholderia spp. (including, but not limited to Burkholderia cepacia)) and ii) the A1 subunit of the labile toxin (LTA1) from enterotoxigenic Escherichia coli or cholera toxin.

In one aspect, disclosed herein are fusion polypeptides of any preceding aspect, wherein the fusion polypeptide is arranged such that the needle tip protein is 5′ of the translocator protein.

Also disclosed herein are fusion polypeptides of any preceding aspect, the needle tip protein and/or translocator protein (including, but not limited to a needle tip protein and translocator protein fusion) is 5′ of LTA1 or the LTA1 is 5′ of the needle tip protein and/or translocator protein (including, but not limited to a needle tip protein and translocator protein fusion).

Also disclosed herein are fusion polypeptides of any preceding aspect, further comprising Pseudomonas spp exolysin A (ExlA), S. marcescens ShlA or Bordetella pertussis FhaC.

Also disclosed herein are vaccines comprising one or more of the fusion polypeptides of any preceding aspect.

In some aspects, disclosed herein are vaccines of any preceding aspect, further comprising MedImmune Emulsion (ME), Chitosan-C48/80 (Chi) nanoparticles, Bacterial Enzymatic Combinatorial Chemistry (BECC) candidate 438 (BECC438), and/or BECC470.

Also disclosed herein are methods of treating, inhibiting, decreasing, reducing, ameliorating, and/or preventing an infection of a Gram negative bacteria (such as, for example, Pseudomonas spp. (including, but not limited to Pseudomonas aeruginosa), Shigella spp, Salmonella enterica, Bordetella spp., Burkholderia spp. (including, but not limited to Burkholderia cepacia)) in a subject comprising administering to the subject the fusion polypeptide or vaccine of any preceding aspect. For example, disclosed herein are methods of treating, inhibiting, decreasing, reducing, ameliorating, and/or preventing an infection of a Gram negative bacteria (such as, for example, Pseudomonas spp. (including, but not limited to Pseudomonas aeruginosa), Shigella spp, Salmonella enterica, Bordetella spp., Burkholderia spp. (including, but not limited to Burkholderia cepacia)) in a subject comprising administering to the subject a therapeutically effective amount of a fusion polypeptides comprising i) a fusion of a needle tip protein (such as, for example, PcrV, IpaD, SseB, Bsp22, LcrV, or BipD) or an antigenic fragment thereof and/or a translocator protein (such as, for example PopB, IpaB, SseC, BopB, YopB, or BipB) or an antigenic fragment thereof from a Type III secretion system (T3SS) of a Gram negative bacteria (such as, for example, Pseudomonas spp. (including, but not limited to Pseudomonas aeruginosa), Shigella spp, Salmonella enterica, Bordetella spp., Yersinia spp., or Burkholderia spp. (including, but not limited to Burkholderia cepacia)) and ii) the A1 subunit of the labile toxin (LTA1) from enterotoxigenic Escherichia coli or cholera toxin or any vaccine comprising said fusion polypeptide. In some aspects, the method further inhibits or prevents colony formation of the bacteria and/or transmission of the bacteria to another subject.

In one aspect, disclosed herein are methods of eliciting an immune response in a subject to a Gram negative bacteria (such as, for example, Pseudomonas spp. (including, but not limited to Pseudomonas aeruginosa), Shigella spp, Salmonella enterica, Bordetella spp., Burkholderia spp. (including, but not limited to Burkholderia cepacia)) comprising administering to the subject a therapeutically effective amount of the fusion polypeptide or vaccine of any preceding aspect. For example, disclosed herein are methods of eliciting an immune response in a subject to a Gram negative bacteria (such as, for example, Pseudomonas spp. (including, but not limited to Pseudomonas aeruginosa), Shigella spp, Salmonella enterica, Bordetella spp., Burkholderia spp. (including, but not limited to Burkholderia cepacia)) comprising administering to the subject a therapeutically effective amount of a fusion polypeptides comprising i) a fusion of a needle tip protein (such as, for example, PcrV, IpaD, SseB, Bsp22, LcrV, or BipD) or an antigenic fragment thereof and/or a translocator protein (such as, for example PopB, IpaB, SseC, BopB, YopB, or BipB) or an antigenic fragment thereof from a Type III secretion system (T3SS) of a Gram negative bacteria (such as, for example, Pseudomonas spp. (including, but not limited to Pseudomonas aeruginosa), Shigella spp, Salmonella enterica, Bordetella spp., Yersinia spp., or Burkholderia spp. (including, but not limited to Burkholderia cepacia)) and ii) the A1 subunit of the labile toxin (LTA1) from enterotoxigenic Escherichia coli or cholera toxin or any vaccine comprising said fusion polypeptide. In some aspects, the immune response comprises a sterilizing immune response.

Also disclosed herein are methods of treating, inhibiting, decreasing, reducing, ameliorating, and/or preventing an opportunistic infection in a subject with cystic fibrosis comprising administering to the subject a therapeutically effective amount of any vaccine or fusion polyeptide of any preceding aspect. For example, disclosed herein are methods of treating, inhibiting, decreasing, reducing, ameliorating, and/or preventing an opportunistic infection in a subject with cystic fibrosis comprising administering to the subject a therapeutically effective amount of a composition comprising a fusion polypeptide comprising i) a fusion of a needle tip protein or an antigenic fragment thereof and/or a translocator protein or an antigenic fragment thereof from a Type III secretion system (T3SS) of Pseudomonas aeruginosa or Burkholderia cepacia and ii) the A1 subunit of the labile toxin (LTA1) from enterotoxigenic Escherichia coli or cholera toxin. In one aspect, the opportunistic infection is a Pseudomonas aeruginosa infection and the tip protein comprises PcrV and the translocator protein comprises PopB. In another aspect, the opportunistic infection comprises a Burkholderia cepacia infection and the tip protein comprises Bsp22 and the translocator protein comprises BopB.

In some aspects, disclosed herein are methods of methods of treating, inhibiting, decreasing, reducing, ameliorating, and/or preventing an infection of a Gram negative bacteria of any preceding aspect; methods of eliciting an immune response of any preceding aspect; and/or methods of treating, inhibiting, decreasing, reducing, ameliorating, and/or preventing an opportunistic infection in a subject with cystic fibrosis of any preceding aspect wherein the fusion polypeptide of the composition is arranged so that the needle tip protein and/or translocator protein (including, but not limited to a needle tip protein and translocator protein fusion) is 5′ of LTA1 or the LTA1 is 5′ of the needle tip protein and/or translocator protein (including, but not limited to a needle tip protein and translocator protein fusion) and/or wherein the composition further comprises Pseudomonas spp exolysin A (ExlA) S. marcescens ShlA or Bordetella pertussis FhaC; and/or wherein the composition further comprises MedImmune Emulsion (ME), Chitosan-C48/80 (Chi) nanoparticles, Bacterial Enzymatic Combinatorial Chemistry (BECC) candidate 438 (BECC438), and/or BECC470.

### III. BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows particle size determined by multi-angle dynamic light scattering (MADLS). (Top) Number weighted size distribution of 0.6 mg/mL L-PaF alone (green), 10,000-fold diluted ME emulsion alone (ME, blue plot), L-PaF+10,000× diluted ME (ME 10\hat{ }4, purple) and L-PaF+1,000× diluted ME (ME 10\hat{ }1, orange). (Bottom) Intensity weighted size distribution of 0.6 mg/mL L-PaF alone (green), 10,000× diluted ME emulsion (blue), L-PaF+10,000× diluted ME (purple) and L-PaF+1,000× diluted ME (orange).

FIGS. 2A, 2B, and 2C show tertiary structure changes in L-PaF monitored in the presence and absence of ME using intrinsic fluorescence spectroscopy. FIG. 2A shows normalized fluorescence spectra of L-PaF and L-PaF with ME at 10° C. Thermal unfolding profiles were monitored by (2B) fluorescence intensity and (2C) MSM peak position. The plots for L-PaF and L-PaF+ME are in red and blue, respectively. The arrow indicates the thermal transition observed for L-PaF when associated with the ME.

FIGS. 3A, 3B, 3C, and 3D show kinetics of serum IgG and IgA. Mice were vaccinated IN in a prime-boost manner with the primary dose on day 0, followed by two booster doses 14 days apart from each other. Blood samples were assessed for the presence of anti-PcrV IgG (3A), IgA (3C) and anti-PopB IgG (3B), IgA (3D) by ELISA. Individual titers were represented as EU/ml. Each point represents the mean while error bars represent the SD of each group (n=10 mice/group).

FIGS. 4A and 4B show kinetics of serum IgG subtypes. Sera from prime-boost IN vaccinated mice were assessed for the presence of different IgG subtypes on day 56 post immunization (4A). Th2/Th1 ratio was assessed as a measure of Th bias (4B). Titers were plotted as EU/ml for the heat map. Each dot in the second panel represents a mean value of {IgG1/(IgG2a+IgG3)} from EU/ml values of a pooled ELISA data.

FIG. 5 shows lung cells secretes IL-17A following antigen-specific ex vivo stimulation. Lung cell suspensions were prepared as described in the text. They were treated with 5 μg/ml of either PcrV or PopB and incubated for 24 h at 37° C. IL-17A secreting cells were enumerated by ELISpot and were plotted as Spots Forming Cells (SFC)/10⁶ cells. Data were plotted as actual values from individuals±SD (n=5) in each group. Statistical significance was calculated by comparing the PBS group with their immunized counterparts using two-way ANOVA (Dunnett's multiple comparison test). *p<0.05, **p<0.01.

FIGS. 6A, 6B, 6C, 6D, 6E, and 6F show that lung cells secretes pro-inflammatory cytokines following stimulation with PcrV or PopB. Lung cell suspensions were prepared and single cell suspensions were treated with 10 μg/ml of either PcrV or PopB and incubated for 48 h at 37° C. Secretion of IL-2 (6A, 6D), IL-6 (6B, 6E) and IL-17A (6C, 6F) were noted as a response of either PcrV or PopB stimulation. Amounts of cytokines were determined by MesoScale Discovery (MSD) analysis as per manufacturer's instructions and were presented as pg/ml/10⁶ cells. Data were plotted as actual values from individuals±SD (n=5) in each group. Statistical significance was calculated by comparing the PBS group with their immunized counterparts using two-way ANOVA (Dunnett's multiple comparison test). *p<0.05, **p<0.01, ***p<0.001.

FIGS. 7A, 7B, 7C, and 7D show in vivo and in vitro protective efficacy studies. For in vivo efficacy studies, on day 56 post immunization, mice were challenged IN with mucoid Pa strain mPa08-31 at a concentration of 4×10⁷ CFU/30 μl/mouse and CFU/lung were determined on 3 DPI (Days Post Infection) (n=4) (7A). The relative lung burdens were compared to PBS and the fold-change was then calculated (7B). For in vitro efficacy studies, an OPK assay was carried out as described in the text. Briefly, the murine macrophage cell line J774A.1 was combined with mPa08-31 at an MOI of 0.1. Heat-inactivated serum was mixed at a ratio of 1:500. OPK activity was determined at 30 min (n=4) (7C), which was then checked for any correlation with in vivo lung burden (7D). OPK was determined using the following formula: (T₀−T₃₀)/T₀×100. T₀ and T₃₀ were CFUs at time 0 and 30 min, respectively. Each dot in (7A) and (7C) represents individual values from either a mouse lung or OPK sample mix. Statistical significance for (7A) and (7C) were calculated as previously explained in FIGS. 5 and 6. Pearson's r coefficient was determined for (7D) to allow assessment of the correlation (r=−0.9538). Simple linear regression was carried out with 95% confidence level (R squared=0.9098, deviation from zero=significant). *p<0.05, ***p<0.001.

FIGS. 8A, 8B, 8C, and 8D show the in vivo post-challenge cytokines were assesed to provide an immune correlate of protection. The effects of post-challenge cytokines were measured in mice lung infected with the Pa isolate mPa08-31. Lung cell suspensions were left untreated for 48 h at 37° C. IL-17A (8A) and TNF-α (8C) were measured as described in FIG. 6. AUC (Area Under Curve) graphs were plotted. Each dot represents a mean±SD values from individual mice (n=5). Statistical significance for these two panels were calculated as described in FIGS. 5 and 6. Correlation of in vivo IL-17A (8B) and in vivo TNF-α (8D) were measured as described in FIG. 7. Pearson's r for these two graphs were −0.5462 and 0.8333, respectively. Significance of a negative and positive r values were described in the text. Simple linear regressions were carried out, where R squared values were 0.2983 and 0.6943, respectively. **p<0.01, ***p<0.001.

FIG. 9 shows current active vaccines. There appears to be one active immunization vaccine, IC43 (OprF/I) Valneva Austria GmbH.

FIG. 10 shows other prophylactic vaccines examined in academia. (Ref 3. Merakou et al).

FIG. 11 shows reduction in lung CFU after challenge. Vaccinated mice were challenged on day 56 with CEC124 (ExlA+T3SS−) or mPA08-31 (ExlA-T3SS+). Three days post-infection lungs were processed and CFU/lung determined.

FIG. 12 shows immune response and Pa burden of CD-1 mice vaccinated with different formulations. Mice were vaccinated as above. Top left: OPK activity in mouse serum from the group vaccinated with the indicated formulation. Top right: CFU/lung burden after challenge with mPA-0831. Bottom left: Levels of IL-17A secreted from lung cells vaccinated with the indicated formulation. Pink indicates lungs were stimulated with PopB while purple indicates stimulation with PcrV. Bottom right: Same as bottom left, but these lungs were collected after challenge and stimulated. The CFU/burden in these lungs is indicated in top right. ***; p≥0.001; **; p≥0.05; *: p≥0.01

FIG. 13 chows the correlation of secreted IL-17A with prevention of infection. Sustained presence of IL-17 (Th17 pre-challenge) prevents the infection (top) but a sudden onset positively correlate with bacterial burden (bottom).

FIG. 14 shows that the chitosan formulation does not protect as well as ME formulation. An alternative to ME formulation, can be a chitosan formulation. Top panel is the CFU burden and percent of PBS control. Bottom left panel is the pre-challenge secreted IL-17A from lung cells after stimulation of cells as indicated. The middle panel is the post-challenged secretion. The right panel is the correlation between the IL-17 (where PopB and PcrV data are added) and CFU/lung.

FIG. 15 shows a survival curve of vaccinated mice after an orally challenged with S. typhimurium.

FIG. 16 shows tissue resident memory cells from the live of mice after vaccination with the indicated treatment. *** p<0.001.

FIG. 17 shows IL-17 or IFNg secreted by hepatocytes after stimulation with L-SseB. *p<0.05; **p<0.01.

FIG. 18 shows a survival curve of vaccinated rabbits after an orally challenged with S. typhimurium.

FIG. 19 shows the correlation of OPK and IL-17 levels with Pa lung burden. Balb/C mice (n=4) were vaccinated intranasally (IN) on days 0, 14 and 28 with PBS or 1 μg L-PaF formulated with ME±BECC438.Left: OPK activity was determined by combining heat-inactivated day 42 sera from the indicated group with mPa08-31 and J774 macrophage cells at an MOI of 0.1 using (T₀−T₃₀)/T₀×100.Middle: On D56, mice were challenged IN mPa08-31 and CFU/lung were determined on D1 post infection (n=4). Right: IL-17 secretion from unstimulated lung cells post-challenge. Pearson's r for protective efficacy correlation of OPK or IL-17 with CFU were −0.9538 and −0.5722, respectively. Groups for the second formulation have been removed for clarity but were used for the r correlation calculations. *p<0.05, **p<0.01, ***p<0.001

FIG. 20 shows structures of the TRL4 agonists MPL, BECC438 and BECC470. Pink boxes indicate major modifications that distinguish BECC from MPL. Note—phosphate moiety lost from BECC470 is on the opposite side of the molecule from MPL. As a control for MPL (GSK), which is not available for research use, 3D-PHAD, a synthetic equivalent for MPL (Avanti Polar Lipids) is normally utilized.

FIGS. 21A, 21B, 21C, and 21D show a homologous influenza challenge protection with a prime/boost vaccination scheme. Top: Balb/C (6-8 weeks old; n=5). Bottom: Balb/C mice (>11 months; n=5). A) Area under the curve (AUC) of serum ELISA. B) Virus titer of lung homogenates 7 days post-infection with 3200 PFU of NL/09. Mice were vaccinated with 40 ng rHA with 100 μg HA (Alhydrogel), or 50 μg PHAD, BECC438 or BECC470.

### IV. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. DEFINITIONS

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

An “increase” can refer to any change that results in a greater amount of a symptom, disease, composition, condition or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purposes. A control can be “positive” or “negative.”

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. COMPOSITIONS

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular needle tip protein (such as, for example, IpaD, SipD, SseB, Bsp22, LcrV, BipD, PcrV, CT053, or CT668), translocator protein (such as, for example, IpaB, SipB, SseC, BopB, YopB, BipB, PopB, CopB, or CopB2), or fusion polypeptide thereof (such as, for example, 22BF, BurkF, PaF, YerF, CT053-CopB, CT053-CopB2, CT668-CopB, or CT668-CopB2) is disclosed and discussed and a number of modifications that can be made to a number of molecules including the needle tip protein (such as, for example, IpaD, SipD, SseB, Bsp22, LcrV, BipD, PcrV, CT053, or CT668), translocator protein (such as, for example, IpaB, SipB, SseC, BopB, YopB, BipB, PopB, CopB, or CopB2), or fusion polypeptide thereof (such as, for example, 22BF, BurkF, PaF, YerF, CT053-CopB, CT053-CopB2, CT668-CopB, or CT668-CopB2) are discussed, specifically contemplated is each and every combination and permutation of needle tip protein (such as, for example, IpaD, SipD, SseB, Bsp22, LcrV, BipD, PcrV, CT053, or CT668), translocator protein (such as, for example, IpaB, SipB, SseC, BopB, YopB, BipB, PopB, CopB, or CopB2), or fusion polypeptide thereof (such as, for example, 22BF, BurkF, PaF, YerF, CT053-CopB, CT053-CopB2, CT668-CopB, or CT668-CopB2) and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

There are no licensed vaccines to prevent Pseudomonas aeruginosa (Pa) infections, but several are in the pipeline. Many of the vaccines, however, only protect against a subset of Pa strains. Like many Gram-negative pathogens, Pa strains of the PAO1/PA14-clades possess a type III secretion system (T3SS), a virulence factor that allows avoidance of host innate immunity and is required for the onset of infection. Structurally resembling a molecular syringe with an external needle, the T3SS apparatus (T3SA) provides an energized conduit from the bacterial cytoplasm into the host cell for transporting effector proteins that mediate key aspects of infection. A needle tip protein and the first of two translocator proteins localize to the distal end of the T3SA needle to mediate host cell contact. These proteins, PcrV and PopB, respectively, are required for pathogenesis and are 95-98% conserved among Pa. Because these are T3SS scaffold proteins required for the early stages of infection for these strains, vaccine escape is reduced due to the fact that mutations in these proteins would impact assembly of an active T3SS and render them nonpathogenic. Indeed, when delivered intranasally (IN), we demonstrated that PcrV+PopB admixed with dmLT (double-mutant labile toxin from Enterotoxigenic E. coli) protected mice against acute Pa pulmonary challenge. To reduce costs associated with their production and formulation, we genetically fused PcrV and PopB to produce a Pa fusion (PaF), which elicited protection against Pa in mouse and rat models. To further reduce the potential reactogenicity associated with IN delivery of dmLT, we fused its A1 subunit (LTA1) to PaF (L-PaF). LTA1 has been shown to stimulate a balanced Th1/Th2/Th17 and mucosal immune response characterized by production of IgA and IL-17A. When delivered IN to mice and rats, L-PaF elicited strong IgG and IgA titers with high levels of opsonophagocytic killing (OPK) activity. It also expedited the serotype independent clearance of Pa from the lungs of the challenged rodents. It has been postulated that high levels of OPK and IL-17A are important for generating a protective immune response in humans against Pa.

In one aspect, disclosed herein are fusion polypeptides comprising i) a fusion of a needle tip protein (such as, for example, PcrV, IpaD, SseB, Bsp22, LcrV, or BipD) or an antigenic fragment thereof and/or a translocator protein (such as, for example PopB, IpaB, SseC, BopB, YopB, or BipB) or an antigenic fragment thereof from a Type III secretion system (T3SS) of a Gram negative bacteria (such as, for example, Pseudomonas spp. (including, but not limited to Pseudomonas aeruginosa), Shigella spp, Salmonella enterica, Bordetella spp., Yersinia spp., or Burkholderia spp. (including, but not limited to Burkholderia cepacia)) and ii) the A1 subunit of the labile toxin (LTA1) from enterotoxigenic Escherichia coli or cholera toxin. Where the needle tip protein and translocator protein are obtained from Pseudomonas spp (i.e., PcrV and PopB fusion), the fusion polypeptide is referred to L-PaF. Similarly, where the needle tip protein and translocator protein are obtained from Shigella spp. (i.e., IpaD and IpaB) the fusion is referred to as L-DBF; a fusion of the Salmonella spp. tip protein and translocator protein (SseB and SseC, respectively) is referred to as L-S2; a fusion of the Bordetella spp. tip protein and translocator protein (Bsp22 and BopB, respectively) is referred to as L-22BF; and a fusion of the Burkholderia spp. tip protein and translocator protein (BipD and BipB, respectively) is referred to as L-BurkF. Also disclosed are compositions such as vaccines comprises said fusion polypeptides.

Recently, Pa outliers have been identified that are devoid of the T3SS entirely and use an ExlA to disrupt host cell membranes. Thus, we have added ExlA (E) to our L-PaF, L-DBF, L-S2, L-22BF, and L-BurkF, which are referred to as L-PaFE, L-DBFE, L-S2E, L-22BFE, and L-BurkFE emulsions, respectively, and the L-PaFE has demonstrated protection in PAO1/14/7 clades when delivered intranasally. In some aspect, ExlA can be substituted with S. marcescens ShlA or Bordetella pertussis FhaC.

Vaccination is perhaps the greatest recent public health achievement and L-PaF represents a unique subunit vaccine platform for preventing Pa infections. Nevertheless, while protective antigens have been identified and shown to be successful as vaccines in mice, they often fail once they are introduced into human trials. Some of these failures are associated with the use of soluble antigens with adjuvants that can elicit a significant immune response in rodents, but do not elicit the same response in humans. In addition to preclinical formulation to develop a stable protein formulation, studies have shown a better response in humans when the antigen is presented as a multimer in the context of a nanoparticle. Perhaps most well-known multimerization method is the use of aluminum salts such as A1 hydrogel, however, the adjuvant activity of aluminum salts tends to skew the immune response to a Th2 response, which is more aligned with the humoral response and not the balanced responses often required for clearing mucosal pathogens. Many nanoparticle formulations are now being tested for use in intramuscular and intranasal routes.

Here we have found that an oil-in-water emulsion as a method to create multimers of L-PaF is superior to the use chitosan nanoparticles. This emulsion contains squalene, which has been shown to promote protection against influenza in an older population. The first formulation examined was an oil-in-water emulsion referred to as ME (MedImmune Emulsion). ME is ~100 nm in size and can thus be taken up directly by dendritic cells. Additionally, we have added the Bacterial Enzymatic Combinatorial Chemistry (BECC) candidate 438 (hereafter referred to as BECC438), a novel TRL-4 agonist that is a bisphosphorylated and detoxified lipid A biosimilar of monophosphoryl lipid A or BECC candidate 470 (BECC470). In addition to fusion with LTA1, use of the BECC (including BECC438 and BECC470) further promotes a balanced Th1-Th2 immune response and increases protection elicited by PaF. Accordingly, in one aspect, disclosed herein are any of the fusion polypeptides, compositions, or vaccines disclosed herein comprising i) a fusion of a needle tip protein (such as, for example, PcrV, IpaD, SseB, Bsp22, LcrV, or BipD) or an antigenic fragment thereof and/or a translocator protein (such as, for example PopB, IpaB, SseC, BopB, YopB, or BipB) or an antigenic fragment thereof from a Type III secretion system (T3SS) of a Gram negative bacteria (such as, for example, Pseudomonas spp. (including, but not limited to Pseudomonas aeruginosa), Shigella spp, Salmonella enterica, Bordetella spp., Yersinia spp., or Burkholderia spp. (including, but not limited to Burkholderia cepacia)) and ii) the A1 subunit of the labile toxin (LTA1) from enterotoxigenic Escherichia coli or cholera toxin, further comprising MedImmune Emulsion (ME), Chitosan-C48/80 (Chi) nanoparticles, Bacterial Enzymatic Combinatorial Chemistry (BECC) candidate 438 (BECC438), and/or BECC470.

It is understood and herein contemplated that a vaccine or composition is not relegated to comprising a fusion protein from a single bacteria spp., but can comprise any combination of fusion polypeptides from one, two, three, four, or all five of Pseudomonas aeruginosa), Shigella spp, Salmonella enterica, Bordetella spp., Burkholderia spp. (including, but not limited to Burkholderia cepacian. Thus, in once aspect, disclosed herein are compositions or vaccines comprising a fusion of a needle tip protein (such as, for example, PcrV and LcrV) and/or a translocator protein (such as, for example PopB and YopB) or an antigenic fragment thereof from Pseudomonas aeruginosa and Burkholderia cepacia) and ii) the A1 subunit of the labile toxin (LTA1) from enterotoxigenic Escherichia coli or cholera toxin. In some aspects, the fusion polypeptide is arranged so that the needle tip protein is 5′ of the translocator protein. In some aspects, the needle tip protein and/or translocator protein (including, but not limited to a needle tip protein and translocator protein fusion) is 5′ of LTA1 or the LTA1 is 5′ of the needle tip protein and/or translocator protein (including, but not limited to a needle tip protein and translocator protein fusion).

In addition, the composition may contain adjuvants, many of which are known in the art. For example, adjuvants suitable for use in the invention include but are not limited to: bacterial or microbial derivatives such as non-toxic derivatives of enterobacterial lipopolysaccharide (LPS), Lipid A derivatives, immunostimulatory oligonucleotides and ADP-ribosylating toxins and detoxified derivatives thereof. Non-toxic derivatives of LPS include monophosphoryl lipid A (MPL) and 3-O-deacylated MPL (3dMPL). 3dMPL is a mixture of three de-O-acylated monophosphoryl lipid A with 4, 5 or 6 acylated chains. A preferred non-toxic derivative of LPS is 3 De-O-acylated monophosphoryl lipid A. Other non-toxic LPS derivatives include monophosphoryl lipid A mimics, such as aminoalkyl glucosaminide phosphate derivatives, e.g. RC-529.

Lipid A derivatives include derivatives of lipid A from Escherichia coli such as OM-174. Immunostimulatory oligonucleotides suitable for use as adjuvants in the invention include nucleotide sequences containing a CpG motif (a dinucleotide sequence containing an unmethylated cytosine linked by a phosphate bond to a guanosine). Double-stranded RNAs and oligonucleotides containing palindromic or poly(dG) sequences have also been shown to be immunostimulatory. The CpG's can include nucleotide modifications/analogs such as phosphorothioate modifications and can be double-stranded or single-stranded, e.g. replacement of guanosine with 2′-deoxy-7-deazaguanosine. The CpG sequence may include, for example, the motif GTCGTT or TTCGTT. The CpG sequence may be specific for inducing a Th1 immune response, such as a CpG-A ODN, or it may be more specific for inducing a B cell response, such a CpG-B ODN, CpG-A and CpG-B ODNs. Preferably, the CpG is a CpG-A ODN. Preferably, the CpG oligonucleotide is constructed so that the 5′ end is accessible for receptor recognition. Optionally, two CpG oligonucleotide sequences may be attached at their 3′ ends to form “immunomers”.

Bacterial ADP-ribosylating toxins and detoxified derivatives thereof may be used as adjuvants in the invention. Preferably, the protein is derived from E. coli (e.g. E. coli heat labile enterotoxin “LT”), cholera (“CT”) (Table 3), or pertussis (“PT”).

TABLE 3
Cholera Toxin (CTA1) subunits and sequences

| Subunit | DNA sequence | AA sequence |
|---|---|---|
| Subunit | ATGGTAAAGATAATATTTGTGTTTTTTATTTT | MVKIIFVFFIFLSSFS |
| A | CTTATCATCATTTTCATATGCAAATGATGATA | YANDDKLYRADSR |
| | AGTTATATCGGGCAGATTCTAGACCTCCTGA | PPDEIKQSGGLMPR |
| | TGAAATAAAGCAGTCAGGTGGTCTTATGCCA | GQSEYFDRGTQMN |
| | AGAGGACAGAGTGAGTACTTTGACCGAGGTA | INLYDHARGTQTGF |
| | CTCAAATGAATATCAACCTTTATGATCATGC | VRHDDGYVSTSISL |
| | AAGAGGAACTCAGACGGGATTTGTTAGGCAC | RSAHLVGQTILSGH |
| | GATGATGGATATGTTTCCACCTCAATTAGTTT | STYYIYVIATAPNM |
| | GAGAAGTGCCCACTTAGTGGGTCAAACTATA | FNVNDVLGAYSPH |
| | TTGTCTGGTCATTCTACTTATTATATATATGT | PDEQEVSALGGIPY |
| | TATAGCCACTGCACCCAACATGTTTAACGTT | SQIYGWYRVHFGV |
| | AATGATGTATTAGGGGCATACAGTCCTCATC | LDEQLHRNRGYRD |
| | CAGATGAACAAGAAGTTTCTGCTTTAGGTGG | RYYSNLDIAPAADG |
| | GATTCCATACTCCCAAATATATGGATGGTAT | YGLAGFPPEHRAW |
| | CGAGTTCATTTTGGGGTGCTTGATGAACAATT | REEPWIHHAPPGCG |
| | ACATCGTAATAGGGGCTACAGAGATAGATAT | NAPRSSMSNTCDE |
| | TACAGTAACTTAGATATTGCTCCAGCAGCAG | KTQSLGVKFLDEY |
| | ATGGTTATGGATTGGCAGGTTTCCCTCCGGA | QSKVKRQIFSGYQS |
| | GCATAGAGCTTGGAGGGAAGAGCCGTGGATT | DIDTHNRIKDEL |
| | CATCATGCACCGCCGGGTTGTGGGAATGCTC | (SEQ ID NO: 16) |
| | CAAGATCATCGATCAGTAATACTTGCGATGA | |
| | AAAAACCCAAAGTCTAGGTGTAAAATTCCTT | |
| | GACGAATACCAATCTAAAGTTAAAAGACAAA | |
| | TATTTTCAGGCTATCAATCTGATATTGATACA | |
| | CATAATAGAATTAAGGATGAATTATGA (SEQ | |
| | ID NO: 15) | |
| Subunit | ATGATTAAATTAAAATTTGGTGTTTTTTTTAC | MIKLKFGVFFTVLL |
| B | AGTTTTACTATCTTCAGCATATGCACATGGAA | SSAYAHGTPQNITD |
| | CACCTCAAAATATTACTGATTTGTGTGCAGA | LCAEYHNTQIYTLN |
| | ATACCACAACACACAAATATATACGCTAAAT | DKIFSYTESLAGKR |
| | GATAAGATATTTTCGTATACAGAATCTCTAG | EMAIITFKNGAIFQ |
| | CTGGAAAAAGAGAGATGGCTATCATTACTTT | VEVPGSQHIDSQKK |
| | TAAGAATGGTGCAATTTTTCAAGTAGAAGTA | AIERMKDTLRIAYL |
| | CCAGGTAGTCAACATATAGATTCACAAAAAA | TEAKVEKLCVWNN |
| | AAGCGATTGAAAGGATGAAGGATACCCTGA | KTPHAIAAISMAN |
| | GGATTGCATATCTTACTGAAGCTAAAGTCGA | (SEQ ID NO: 18) |
| | AAAGTTATGTGTATGGAATAATAAAACGCCT | |
| | CATGCGATTGCCGCAATTAGTATGGCAAATT | |
| | AA (SEQ ID NO: 17) | |

### 1. Sequence Similarities

It is understood that as discussed herein the use of the terms homology and identity mean the same thing as similarity. Thus, for example, if the use of the word homology is used between two non-natural sequences it is understood that this is not necessarily indicating an evolutionary relationship between these two sequences, but rather is looking at the similarity or relatedness between their nucleic acid sequences. Many of the methods for determining homology between two evolutionarily related molecules are routinely applied to any two or more nucleic acids or proteins for the purpose of measuring sequence similarity regardless of whether they are evolutionarily related or not.

In general, it is understood that one way to define any known variants and derivatives or those that might arise, of the disclosed genes and proteins herein, is through defining the variants and derivatives in terms of homology to specific known sequences. This identity of particular sequences disclosed herein is also discussed elsewhere herein. In general, variants of genes and proteins herein disclosed (such as, for example, Bsp22, LcrV, BipD, PcrV, CT053, CT668, BopB, YopB, BipB, PopB, CopB, CopB2, 22BF, BurkF, PaF, YerF, CT053-CopB, CT053-CopB2, CT668-CopB, or CT668-CopB2) typically have at least about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to the stated sequence or the native sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Nat. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, WI), or by inspection.

It is understood that any of the methods typically can be used and that in certain instances the results of these various methods may differ, but the skilled artisan understands if identity is found with at least one of these methods, the sequences would be said to have the stated identity, and be disclosed herein.

For example, as used herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages).

### 2. Nucleic Acids

There are a variety of molecules disclosed herein that are nucleic acid based, including for example the nucleic acids that encode, for example Bsp22, LcrV, BipD, PcrV, CT053, CT668, BopB, YopB, BipB, PopB, CopB, CopB2, 22BF, BurkF, PaF, YerF, CT053-CopB, CT053-CopB2, CT668-CopB, or CT668-CopB2 or antigenic fragments thereof, as well as various functional nucleic acids. The disclosed nucleic acids are made up of for example, nucleotides, nucleotide analogs, or nucleotide substitutes. Non-limiting examples of these and other molecules are discussed herein. It is understood that for example, when a vector is expressed in a cell, that the expressed mRNA will typically be made up of A, C, G, and U. Likewise, it is understood that if, for example, an antisense molecule is introduced into a cell or cell environment through for example exogenous delivery, it is advantageous that the antisense molecule be made up of nucleotide analogs that reduce the degradation of the antisense molecule in the cellular environment.

### a) Nucleotides and Related Molecules

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. An non-limiting example of a nucleotide would be 3′-AMP (3′-adenosine monophosphate) or 5′-GMP (5′-guanosine monophosphate). There are many varieties of these types of molecules available in the art and available herein.

A nucleotide analog is a nucleotide which contains some type of modification to either the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine, as well as modifications at the sugar or phosphate moieties. There are many varieties of these types of molecules available in the art and available herein.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid. There are many varieties of these types of molecules available in the art and available herein.

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety. (Letsinger et al., Proc. Natl. Acad. Sci. USA, 1989, 86, 6553-6556). There are many varieties of these types of molecules available in the art and available herein.

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

### b) Sequences

There are a variety of sequences related to the protein molecules involved in the signaling pathways disclosed herein, for example Bsp22, LcrV, BipD, PcrV, CT053, CT668, BopB, YopB, BipB, PopB, CopB, CopB2, 22BF, BurkF, PaF, YerF, CT053-CopB, CT053-CopB2, CT668-CopB, or CT668-CopB2, or any of the nucleic acids disclosed herein for making Bsp22, LcrV, BipD, PcrV, CT053, CT668, BopB, YopB, BipB, PopB, CopB, CopB2, 22BF, BurkF, PaF, YerF, CT053-CopB, CT053-CopB2, CT668-CopB, or CT668-CopB2, all of which are encoded by nucleic acids or are nucleic acids. The sequences for the human analogs of these genes, as well as other analogs, and alleles of these genes, and splice variants and other types of variants, are available in a variety of protein and gene databases, including GENBANK®. Those of skill in the art understand how to resolve sequence discrepancies and differences and to adjust the compositions and methods relating to a particular sequence to other related sequences. Primers and/or probes can be designed for any given sequence given the information disclosed herein and known in the art.

### 3. Nucleic Acid Delivery

In the methods described above which include the administration and uptake of exogenous DNA into the cells of a subject (i.e., gene transduction or transfection), the disclosed nucleic acids can be in the form of naked DNA or RNA, or the nucleic acids can be in a vector for delivering the nucleic acids to the cells, whereby the antibody-encoding DNA fragment is under the transcriptional regulation of a promoter, as would be well understood by one of ordinary skill in the art. The vector can be a commercially available preparation, such as an adenovirus vector (Quantum Biotechnologies, Inc. (Laval, Quebec, Canada). Delivery of the nucleic acid or vector to cells can be via a variety of mechanisms. As one example, delivery can be via a liposome, using commercially available liposome preparations such as LIPOFECTIN, LIPOFECTAMINE (GIBCO-BRL, Inc., Gaithersburg, MD), SUPERFECT (Qiagen, Inc. Hilden, Germany) and TRANSFECTAM (Promega Biotec, Inc., Madison, WI), as well as other liposomes developed according to procedures standard in the art. In addition, the disclosed nucleic acid or vector can be delivered in vivo by electroporation, the technology for which is available from Genetronics, Inc. (San Diego, CA) as well as by means of a SONOPORATION machine (ImaRx Pharmaceutical Corp., Tucson, AZ).

As one example, vector delivery can be via a viral system, such as a retroviral vector system which can package a recombinant retroviral genome (see e.g., Pastan et al., Proc. Natl. Acad. Sci. U.S.A. 85:4486, 1988; Miller et al., Mol. Cell. Biol. 6:2895, 1986). The recombinant retrovirus can then be used to infect and thereby deliver to the infected cells nucleic acid encoding a broadly neutralizing antibody (or active fragment thereof). The exact method of introducing the altered nucleic acid into mammalian cells is, of course, not limited to the use of retroviral vectors. Other techniques are widely available for this procedure including the use of adenoviral vectors (Mitani et al., Hum. Gene Ther. 5:941-948, 1994), adeno-associated viral (AAV) vectors (Goodman et al., Blood 84:1492-1500, 1994), lentiviral vectors (Naidini et al., Science 272:263-267, 1996), pseudotyped retroviral vectors (Agrawal et al., Exper. Hematol. 24:738-747, 1996). Physical transduction techniques can also be used, such as liposome delivery and receptor-mediated and other endocytosis mechanisms (see, for example, Schwartzenberger et al., Blood 87:472-478, 1996). This disclosed compositions and methods can be used in conjunction with any of these or other commonly used gene transfer methods.

As one example, if the antibody-encoding nucleic acid is delivered to the cells of a subject in an adenovirus vector, the dosage for administration of adenovirus to humans can range from about 10⁷ to about 10⁹ plaque forming units (pfu) per injection but can be as high as about 10¹² pfu per injection (Crystal, Hum. Gene Ther. 8:985-1001, 1997; Alvarez and Curiel, Hum. Gene Ther. 8:597-613, 1997). A subject can receive a single injection, or, if additional injections are necessary, they can be repeated at six month intervals (or other appropriate time intervals, as determined by the skilled practitioner) for an indefinite period and/or until the efficacy of the treatment has been established.

Parenteral administration of the nucleic acid or vector, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. For additional discussion of suitable formulations and various routes of administration of therapeutic compounds, see, e.g., Remington: The Science and Practice of Pharmacy (19th ed.) ed. A. R. Gennaro, Mack Publishing Company, Easton, PA 1995.

### 4. Delivery of the Compositions to Cells

There are a number of compositions and methods which can be used to deliver nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991). Such methods are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier.

### a) Nucleic Acid Based Delivery Systems

Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)).

As used herein, plasmid or viral vectors are agents that transport the disclosed nucleic acids, such as 22BF into the cell without degradation and include a promoter yielding expression of the gene in the cells into which it is delivered. Viral vectors are, for example, Adenovirus, Adeno-associated virus, Herpes virus, Vaccinia virus, Polio virus, AIDS virus, neuronal trophic virus, Sindbis and other RNA viruses, including these viruses with the HIV backbone. Also preferred are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A preferred embodiment is a viral vector which has been engineered so as to suppress the immune response of the host organism, elicited by the viral antigens. Preferred vectors of this type will carry coding regions for Interleukin 8 or 10.

Viral vectors can have higher transaction (ability to introduce genes) abilities than chemical or physical methods to introduce genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promotor cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

### (1) Retroviral Vectors

A retrovirus is an animal virus belonging to the virus family of Retroviridae, including any types, subfamilies, genus, or tropisms. Retroviral vectors, in general, are described by Verma, I. M., Retroviral vectors for gene transfer.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome, contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serve as the priming site for the synthesis of the second strand of DNA synthesis, and specific sequences near the ends of the LTRs that enable the insertion of the DNA state of the retrovirus to insert into the host genome. The removal of the gag, pol, and env genes allows for about 8 kb of foreign sequence to be inserted into the viral genome, become reverse transcribed, and upon replication be packaged into a new retroviral particle. This amount of nucleic acid is sufficient for the delivery of a one to many genes depending on the size of each transcript. It is preferable to include either positive or negative selectable markers along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

### (2) Adenoviral Vectors

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J Neuroscience 5:1287-1291 (1993); and Ragot, J. Gen. Virology 74:501-507 (1993)). Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol. 4:1528-1533 (1984); Varga et al., J. Virology 65:6061-6070 (1991); Wickham et al., Cell 73:309-319 (1993)).

A viral vector can be one based on an adenovirus which has had the E1 gene removed and these virons are generated in a cell line such as the human 293 cell line. In another preferred embodiment both the E1 and E3 genes are removed from the adenovirus genome.

### 5. Adeno-Associated Viral Vectors

Another type of viral vector is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1 C vector produced by Avigen, San Francisco, CA, which can contain the herpes simplex virus thymidine kinase gene, HSV-tk, and/or a marker gene, such as the gene encoding the green fluorescent protein, GFP.

In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus.

Typically the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorporated by reference for material related to the AAV vector.

The disclosed vectors thus provide DNA molecules which are capable of integration into a mammalian chromosome without substantial toxicity.

The inserted genes in viral and retroviral usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

### a) Large Payload Viral Vectors

Molecular genetic experiments with large human herpesviruses have provided a means whereby large heterologous DNA fragments can be cloned, propagated and established in cells permissive for infection with herpesviruses (Sun et al., Nature genetics 8: 33-41, 1994; Cotter and Robertson, Curr Opin Mol Ther 5: 633-644, 1999). These large DNA viruses (herpes simplex virus (HSV) and Epstein-Barr virus (EBV), have the potential to deliver fragments of human heterologous DNA>150 kb to specific cells. EBV recombinants can maintain large pieces of DNA in the infected B-cells as episomal DNA. Individual clones carried human genomic inserts up to 330 kb appeared genetically stable The maintenance of these episomes requires a specific EBV nuclear protein, EBNA1, constitutively expressed during infection with EBV. Additionally, these vectors can be used for transfection, where large amounts of protein can be generated transiently in vitro. Herpesvirus amplicon systems are also being used to package pieces of DNA>220 kb and to infect cells that can stably maintain DNA as episomes.

Other useful systems include, for example, replicating and host-restricted non-replicating vaccinia virus vectors.

### b) Non-Nucleic Acid Based Systems

The disclosed compositions can be delivered to the target cells in a variety of ways. For example, the compositions can be delivered through electroporation, or through lipofection, or through calcium phosphate precipitation. The delivery mechanism chosen will depend in part on the type of cell targeted and whether the delivery is occurring for example in vivo or in vitro.

Thus, the compositions can comprise, in addition to the disclosed needle tip protein-translocator protein fusion (such as, for example, 22BF) or vectors for example, lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a compound and a cationic liposome can be administered to the blood afferent to a target organ or inhaled into the respiratory tract to target cells of the respiratory tract. Regarding liposomes, see, e.g., Brigham et al. Am. J. Resp. Cell. Mol. Biol. 1:95-100 (1989); Felgner et al. Proc. Natl. Acad. Sci USA 84:7413-7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

In the methods described above which include the administration and uptake of exogenous DNA into the cells of a subject (i.e., gene transduction or transfection), delivery of the compositions to cells can be via a variety of mechanisms. As one example, delivery can be via a liposome, using commercially available liposome preparations such as LIPOFECTIN, LIPOFECTAMINE (GIBCO-BRL, Inc., Gaithersburg, MD), SUPERFECT (Qiagen, Inc. Hilden, Germany) and TRANSFECTAM (Promega Biotec, Inc., Madison, WI), as well as other liposomes developed according to procedures standard in the art. In addition, the disclosed nucleic acid or vector can be delivered in vivo by electroporation, the technology for which is available from Genetronics, Inc. (San Diego, CA) as well as by means of a SONOPORATION machine (ImaRx Pharmaceutical Corp., Tucson, AZ).

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J Cancer, 60:275-281, (1989); Bagshawe, et al., Br. J Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). These techniques can be used for a variety of other specific cell types. Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

Nucleic acids that are delivered to cells which are to be integrated into the host cell genome, typically contain integration sequences. These sequences are often viral related sequences, particularly when viral based systems are used. These viral integration systems can also be incorporated into nucleic acids which are to be delivered using a non-nucleic acid based system of deliver, such as a liposome, so that the nucleic acid contained in the delivery system can become integrated into the host genome.

Other general techniques for integration into the host genome include, for example, systems designed to promote homologous recombination with the host genome. These systems typically rely on sequence flanking the nucleic acid to be expressed that has enough homology with a target sequence within the host cell genome that recombination between the vector nucleic acid and the target nucleic acid takes place, causing the delivered nucleic acid to be integrated into the host genome. These systems and the methods necessary to promote homologous recombination are known to those of skill in the art.

### c) In Vivo/Ex Vivo

As described above, the compositions can be administered in a pharmaceutically acceptable carrier and can be delivered to the subject's cells in vivo and/or ex vivo by a variety of mechanisms well known in the art (e.g., uptake of naked DNA, liposome fusion, intramuscular injection of DNA via a gene gun, endocytosis and the like).

If ex vivo methods are employed, cells or tissues can be removed and maintained outside the body according to standard protocols well known in the art. The compositions can be introduced into the cells via any gene transfer mechanism, such as, for example, calcium phosphate mediated gene delivery, electroporation, microinjection or proteoliposomes. The transduced cells can then be infused (e.g., in a pharmaceutically acceptable carrier) or homotopically transplanted back into the subject per standard methods for the cell or tissue type. Standard methods are known for transplantation or infusion of various cells into a subject.

### 6. Expression Systems

The nucleic acids that are delivered to cells typically contain expression controlling systems. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

### a) Viral Promoters and Enhancers

Preferred promoters controlling transcription from vectors in mammalian host cells may be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g. beta actin promoter. The early and late promoters of the SV40 virus are conveniently obtained as an SV40 restriction fragment which also contains the SV40 viral origin of replication (Fiers et al., Nature, 273: 113 (1978)). The immediate early promoter of the human cytomegalovirus is conveniently obtained as a HindIII E restriction fragment (Greenway, P. J. et al., Gene 18: 355-360 (1982)). Of course, promoters from the host cell or related species also are useful herein.

Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell Bio. 3: 1108 (1983)) to the transcription unit. Furthermore, enhancers can be within an intron (Banerji, J. L. et al., Cell 33: 729 (1983)) as well as within the coding sequence itself (Osborne, T. F., et al., Mol. Cell Bio. 4: 1293 (1984)). They are usually between 10 and 300 bp in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, α-fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

The promotor and/or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

In certain embodiments the promoter and/or enhancer region can act as a constitutive promoter and/or enhancer to maximize expression of the region of the transcription unit to be transcribed. In certain constructs the promoter and/or enhancer region be active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTR.

It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contains a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases. It is also preferred that the transcribed units contain other standard sequences alone or in combination with the above sequences improve expression from, or stability of, the construct.

### b) Markers

The viral vectors can include nucleic acid sequence encoding a marker product. This marker product is used to determine if the gene has been delivered to the cell and once delivered is being expressed. Preferred marker genes are the E. Coli lacZ gene, which encodes ß-galactosidase, and green fluorescent protein.

In some embodiments the marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO DHFR− cells and mouse LTK− cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puromycin.

### 7. Peptides

### a) Protein Variants

As discussed herein there are numerous variants of the needle tip protein-translocator protein fusion (such as, for example, Bsp22, LcrV, BipD, PcrV, CT053, CT668, BopB, YopB, BipB, PopB, CopB, CopB2, 22BF, BurkF, PaF, YerF, CT053-CopB, CT053-CopB2, CT668-CopB, or CT668-CopB2) that are known and herein contemplated. In addition, to the known functional strain variants there are derivatives of the needle tip protein and translocator protein which also function in the disclosed methods and compositions. Protein variants and derivatives are well understood to those of skill in the art and can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than from about 2 to about 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of from about 1 to about 10 amino acid residues; and deletions will range from about 1 to about 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 4 and 5 and are referred to as conservative substitutions.

TABLE 4
Amino Acid Abbreviations

| | |
|---|---|
| Amino Acid | Abbreviations |

| | | |
|---|---|---|
| Alanine | Ala | A |
| Allosoleucine | AIle | |
| Arginine | Arg | R |
| Asparagine | Asn | N |
| Aspartic acid | Asp | D |
| Cysteine | Cys | C |
| Glutamic acid | Glu | E |
| Glutamine | Gln | Q |
| Glycine | Gly | G |
| Histidine | His | H |
| Isolelucine | Ile | I |
| Leucine | Leu | L |
| Lysine | Lys | K |
| Phenylalanine | Phe | F |
| Proline | Pro | P |
| Pyroglutamic acid | pGlu | |
| Serine | Ser | S |
| Threonine | Thr | T |
| Tyrosine | Tyr | Y |
| Tryptophan | Trp | W |
| Valine | Val | V |

TABLE 5
Amino Acid Substitutions
Original Residue Exemplary Conservative Substitutions,
others are known in the art.

| | |
|---|---|
| Ala | Ser |
| Arg | Lys; Gln |
| Asn | Gln; His |
| Asp | Glu |
| Cys | Ser |
| Gln | Asn, Lys |
| Glu | Asp |
| Gly | Pro |
| His | Asn; Gln |
| Ile | Leu; Val |
| Leu | Ile; Val |
| Lys | Arg; Gln |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Substantial changes in function or immunological identity are made by selecting substitutions that are less conservative than those in Table 4, i.e., selecting residues that differ more significantly in their effect on maintaining (a) the structure of the polypeptide backbone in the area of the substitution, for example as a sheet or helical conformation, (b) the charge or hydrophobicity of the molecule at the target site, or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; (d) a residue having a bulky side chain, e.g., phenylalanine, is substituted for (or by) one not having a side chain, e.g., glycine, in this case, or (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

It is understood that one way to define the variants and derivatives of the disclosed proteins herein is through defining the variants and derivatives in terms of homology/identity to specific known sequences. For example, SEQ ID NO: 1 sets forth a particular sequence of Bordetella needle tip protein-translocator protein fusion (22BF) and SEQ ID NO: 2 sets forth a particular sequence of a 22BF fusion protein. Specifically disclosed are variants of these and other proteins herein disclosed which have at least, 70% or 75% or 80% or 85% or 90% or 95% homology to the stated sequence. Those of skill in the art readily understand how to determine the homology of two proteins. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, WI), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989.

It is understood that the description of conservative mutations and homology can be combined together in any combination, such as embodiments that have at least 70% homology to a particular sequence wherein the variants are conservative mutations.

As this specification discusses various proteins and protein sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence. For example, one of the many nucleic acid sequences that can encode the protein sequence set forth in SEQ ID NO: 2 is set forth in SEQ ID NO: 1. It is understood that for this mutation all of the nucleic acid sequences that encode this particular derivative of the 22BF are also disclosed. It is also understood that while no amino acid sequence indicates what particular DNA sequence encodes that protein within an organism, where particular variants of a disclosed protein are disclosed herein, the known nucleic acid sequence that encodes that protein in the particular needle tip protein-translocator protein fusion (such as, for example, 22BF) from which that protein arises is also known and herein disclosed and described.

It is understood that there are numerous amino acid and peptide analogs which can be incorporated into the disclosed compositions. For example, there are numerous D amino acids or amino acids which have a different functional substituent then the amino acids shown in Table 4 and Table 5. The opposite stereo isomers of naturally occurring peptides are disclosed, as well as the stereo isomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site specific way.

Molecules can be produced that resemble peptides, but which are not connected via a natural peptide linkage. For example, linkages for amino acids or amino acid analogs can include —CH₂NH—, —CH₂S—, —CH₂—CH₂—, —CH═CH— (cis and trans), —COCH₂—, —CH(OH)CH₂—, and —CHH₂SO— (These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci (1980) pp. 463-468; Hudson, D. et al., Int J Pept Prot Res 14:177-185 (1979) (—CH₂NH—, —CH₂CH₂—); Spatola et al. Life Sci 38:1243-1249 (1986) (—CH₂—S); Hann J. Chem. Soc Perkin Trans. 1307-314 (1982) (—CH═CH—, cis and trans); Almquist et al. J. Med. Chem. 23:1392-1398 (1980) (—COCH₂—); Jennings-White et al. Tetrahedron Lett 23:2533 (1982) (—COCH₂—); Szelke et al. European Appln, EP 45665 CA (1982): 97:39405 (1982) (—CH(OH)CH₂—); Holladay et al. Tetrahedron. Lett 24:4401-4404 (1983) (—C(OH)CH₂—); and Hruby Life Sci 31:189-199 (1982) (—CH₂—S—); each of which is incorporated herein by reference. A particularly preferred non-peptide linkage is —CH₂NH—. It is understood that peptide analogs can have more than one atom between the bond atoms, such as β-alanine, γ-aminobutyric acid, and the like.

Amino acid analogs and analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations.

### 8. Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J Cancer, 60:275-281, (1989); Bagshawe, et al., Br. J Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

### a) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, PA 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, more preferably from about 7 to about 7.6, and most preferably about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, anti-inflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, tri-alkyl and aryl amines and substituted ethanolamines.

### b) Therapeutic Uses

Effective dosages and schedules for administering the compositions may be determined empirically, and making such determinations is within the skill in the art. The dosage ranges for the administration of the compositions are those large enough to produce the desired effect in which the symptoms of the disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

In a preferred embodiment, the amount of protein that is administered per dose of vaccine is in the range of from about 0.0001 to about 1000 μg/kg. In one embodiment, the amount is in the range of from about 0.001 to about 1000 μg/kg of body weight of the recipient. In one embodiment, the amount is in the range of from about 0.01 to about 1000 μg/kg of body weight of the recipient. In one embodiment, the amount is in the range of from about 0.01 to about 100 μg/kg of body weight of the recipient. Those of skill in the art will recognize that the precise dosage may vary from situation to situation and from patient to patient, depending on e.g. age, gender, overall health, various genetic factors, and other variables known to those of skill in the art. Dosages are typically determined e.g. in the course of animal and/or human clinical trials as conducted by skilled medical personnel, e.g. physicians or veterinarians.

### C. METHODS OF USING THE COMPOSITIONS

Herein, the protective efficacy of the Bordetella spp. tip/translocator fusion, 22BF, is examined against lethal lung challenge and with complete (sterilizing) clearance of colonizing bacteria. Unlike some components of the current aP vaccine, Bsp22 and BopB are required for infection and are not mutable since they must be retained structurally and functionally within the context of a large nanomachine residing within the Bordetella cell envelope. Furthermore, targeting the Bordetella T3SA renders the pathogen less able to fight off the host innate and adaptive immune responses. Regardless of whether 22BF is protective alone or when used with components of the current aP vaccine, the innovation of this high risk, high reward investigation lies in whether this subunit vaccine can elicit sterilizing immunity and thereby prevent the colonization that results in host to host transmission. It has been reported that Bsp22 (a component of the 22BF fusion vaccine) does not elicit a serum antibody response in humans during the course of natural infection and is not a protective antigen in mice. Nevertheless, as shown herein, protective and sterilizing immunity can be obtained with the compositions disclosed herein.

Thus, in one aspect, disclosed herein are methods of eliciting an immune response in a subject to a Gram negative bacteria (such as, for example, Bordetella spp., Burkholderia spp., Chlamydia spp., Pseudomonas spp., Shigella spp., Salmonella spp., Vibrio spp. Enteropathogenic or Enterohemorrhagic E. coli or Yersinia spp.) comprising administering to the subject the fusion polypeptides, compositions, or vaccines disclosed herein. Accordingly, in one aspect, disclosed herein are methods of eliciting an immune response in a subject to a Gram negative bacteria (such as, for example, Pseudomonas spp. (including, but not limited to Pseudomonas aeruginosa), Shigella spp, Salmonella enterica, Bordetella spp., Yersinia spp., or Burkholderia spp. (including, but not limited to Burkholderia cepacia)) comprising administering to the subject a therapeutically effective amount of a fusion polypeptides comprising i) a fusion of a needle tip protein (such as, for example, PcrV, IpaD, SseB, Bsp22, LcrV, or BipD) or an antigenic fragment thereof and/or a translocator protein (such as, for example PopB, IpaB, SseC, BopB, YopB, or BipB) or an antigenic fragment thereof from a Type III secretion system (T3SS) of a Gram negative bacteria (such as, for example, Pseudomonas spp. (including, but not limited to Pseudomonas aeruginosa), Shigella spp, Salmonella enterica, Bordetella spp., Yersinia spp., or Burkholderia spp. (including, but not limited to Burkholderia cepacia)) and ii) the A1 subunit of the labile toxin (LTA1) from enterotoxigenic Escherichia coli or cholera toxin or any vaccine comprising said fusion polypeptide. In some aspects, the Gram negative bacteria is not a Shigella spp. or Salmonella spp. In one aspect, the immune response elicited provides sterilizing immunity to the infectious bacterium.

As can be appreciated by the skilled artisan, the methods of eliciting an immune response can be used for the purpose of treating, inhibiting, or preventing an infection of a Gram negative bacteria (such as, for example, Shigella spp, Salmonella enterica, Bordetella spp., Burkholderia spp., Chlamydia spp., Pseudomonas spp., Vibrio spp. Enteropathogenic or Enterohemorrhagic E. coli or Yersinia spp). Thus, in one aspect, disclosed herein are methods of treating, inhibiting, decreasing, reducing, ameliorating, and/or preventing an infection of a Gram negative bacteria (such as, for example, Pseudomonas spp. (including, but not limited to Pseudomonas aeruginosa), Shigella spp, Salmonella enterica, Bordetella spp., Burkholderia spp. (including, but not limited to Burkholderia cepacia)) in a subject comprising administering to the subject any of the fusion polypeptides or vaccines disclosed herein. For example, disclosed herein are methods of treating, inhibiting, decreasing, reducing, ameliorating, and/or preventing an infection of a Gram negative bacteria (such as, for example, Pseudomonas spp. (including, but not limited to Pseudomonas aeruginosa), Shigella spp, Salmonella enterica, Bordetella spp., Yersinia spp., or Burkholderia spp. (including, but not limited to Burkholderia cepacia)) in a subject comprising administering to the subject a therapeutically effective amount of a fusion polypeptides comprising i) a fusion of a needle tip protein (such as, for example, PcrV, IpaD, SseB, Bsp22, LcrV, or BipD) or an antigenic fragment thereof and/or a translocator protein (such as, for example PopB, IpaB, SseC, BopB, YopB, or BipB) or an antigenic fragment thereof from a Type III secretion system (T3SS) of a Gram negative bacteria (such as, for example, Pseudomonas spp. (including, but not limited to Pseudomonas aeruginosa), Shigella spp, Salmonella enterica, Bordetella spp., Yersinia spp., or Burkholderia spp. (including, but not limited to Burkholderia cepacia)) and ii) the A1 subunit of the labile toxin (LTA1) from enterotoxigenic Escherichia coli or cholera toxin or any vaccine comprising said fusion polypeptide. In some aspects, the method further inhibits or prevents colony formation of the bacteria and/or transmission of the bacteria to another subject.

It is understood and herein contemplated that patients with cystic fibrosis can be susceptible to opportunistic infections. Thus, also disclosed herein are methods of treating, inhibiting, decreasing, reducing, ameliorating, and/or preventing an opportunistic infection in a subject with cystic fibrosis comprising administering to the subject a therapeutically effective amount of any of the vaccines or fusion polypeptides disclosed herein. For example, disclosed herein are methods of treating, inhibiting, decreasing, reducing, ameliorating, and/or preventing an opportunistic infection in a subject with cystic fibrosis comprising administering to the subject a therapeutically effective amount of a composition comprising a fusion polypeptide comprising i) a fusion of a needle tip protein or an antigenic fragment thereof and/or a translocator protein or an antigenic fragment thereof from a Type III secretion system (T3SS) of Pseudomonas aeruginosa or Burkholderia cepacia and ii) the A1 subunit of the labile toxin (LTA1) from enterotoxigenic Escherichia coli or cholera toxin. In one aspect, the opportunistic infection is a Pseudomonas aeruginosa infection and the tip protein comprises PcrV and the translocator protein comprises PopB. In another aspect, the opportunistic infection comprises a Burkholderia cepacia infection and the tip protein comprises Bsp22 and the translocator protein comprises BopB.

In some aspects, disclosed herein are methods of methods of treating, inhibiting, decreasing, reducing, ameliorating, and/or preventing an infection of a Gram negative bacteria; methods of eliciting an immune response; and/or methods of treating, inhibiting, decreasing, reducing, ameliorating, and/or preventing an opportunistic infection in a subject with cystic fibrosis wherein the fusion polypeptide of the composition is arranged so that the needle tip protein and/or translocator protein (including, but not limited to a needle tip protein and translocator protein fusion) is 5′ of LTA1 or the LTA1 is 5′ of the needle tip protein and/or translocator protein (including, but not limited to a needle tip protein and translocator protein fusion) and/or wherein the composition further comprises Pseudomonas spp exolysin A (ExlA), S. marcescens ShlA or Bordetella pertussis FhaC; and/or wherein the composition further comprises MedImmune Emulsion (ME), Chitosan-C48/80 (Chi) nanoparticles, Bacterial Enzymatic Combinatorial Chemistry (BECC) candidate 438 (BECC438), and/or BECC470.

### D. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1: Effect of Two Unique Nanoparticle Formulations on the Efficacy of a Broadly Protective Vaccine Against Pseudomonas aeruginosa

### a) Results and Discussion

(1) L-PaF Interacts with ME

The first formulation examined was an oil-in-water emulsion referred to as ME (MedImmune Emulsion). ME is ~100 nm in size and can thus be taken up directly by dendritic cells. We determined that L-PaF was associated with ME by measuring the particle size distribution and zeta potential of ME before and after mixing with L-PaF. The zeta potential of ME mixed with L-PaF had a slight positive value compared to the negative values of ME or L-PaF alone, which indicates that their association significantly alters the resulting particle's surface features (Table 1). With respect to particle size, L-PaF had a high polydispersity index (PdI>80%) with a size distribution ranging from about 8 to 400 nm, indicating that L-PaF has the tendency to form aggregates (Table 1). Conversely, ME had a unimodal size distribution with a low polydispersity index (PdI<10%) (Table 1). Mixing L-PaF with ME increased the particle size from 113 nm to 143 nm without an increase in polydispersity (PdI<10%) (Table 1). In other work, it was found that the addition of BECC did not significantly change the size of the ME.

TABLE 1
Summary table of particle characteristics
of L-PaF, ME and L-PaF + ME.

| | Zeta | | Poly- |
|---|---|---|---|
| Name | potential (mV) | Z_average | disperity (%) |
| ME | −23.4 ± 1.6 | 113.3 ± 0.6 | 9.1 ± 1.6 |
| L-PaF | −6.31 ± 0.23 | 35.9 ± 4.1 | 83.5 ± 5.7 |
| ME + L-PaF | 1.47 ± 0.8 | 144.6 ± 2.9 | 8.1 ± 1.3 |

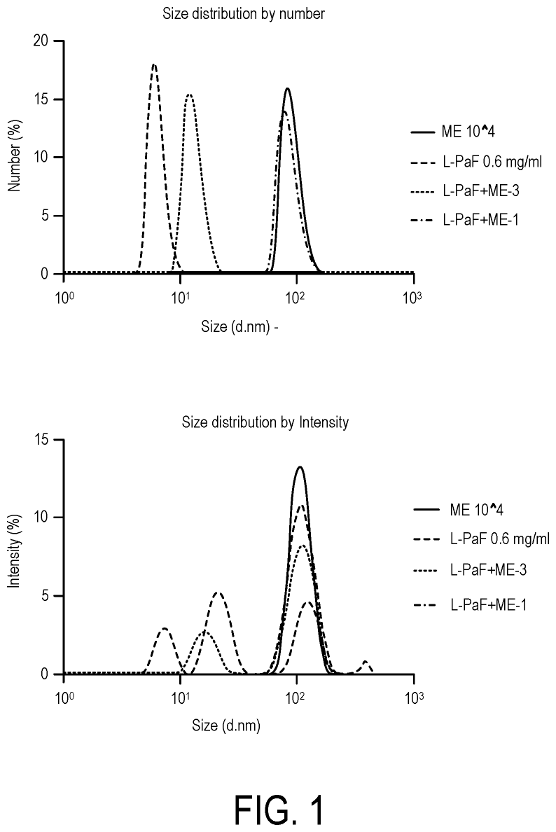

To investigate the change of particle size with the addition of L-PaF to ME further, we performed MADLS measurements (FIG. 1). These results confirmed that L-PaF is heterogeneous with four distinct particle size populations based on intensity-based measurements, however particle numbers indicated that the smallest species (~8 nm) was dominant. Meanwhile, ME is largely homogenous in size with an ~100 nm particle size. When 10,000× diluted ME was mixed with L-PaF, there were two populations of particle size based on intensity (FIG. 1, top). One population was ~10 nm close to the size of L-PaF alone and the other was ~100 nm, indicating not all L-PaF was incorporated into ME particle (FIG. 1). In contrast, when 1,000× diluted ME was mixed with L-PaF, only one population of particle size was observed for both based on number and intensity (FIG. 1, bottom). The results confirmed that the mixing of L-PaF with ME leads to the incorporation of L-PaF into the ME particle with an excess of ME leading to a single homogenous population L-PaF/ME nanoparticles.

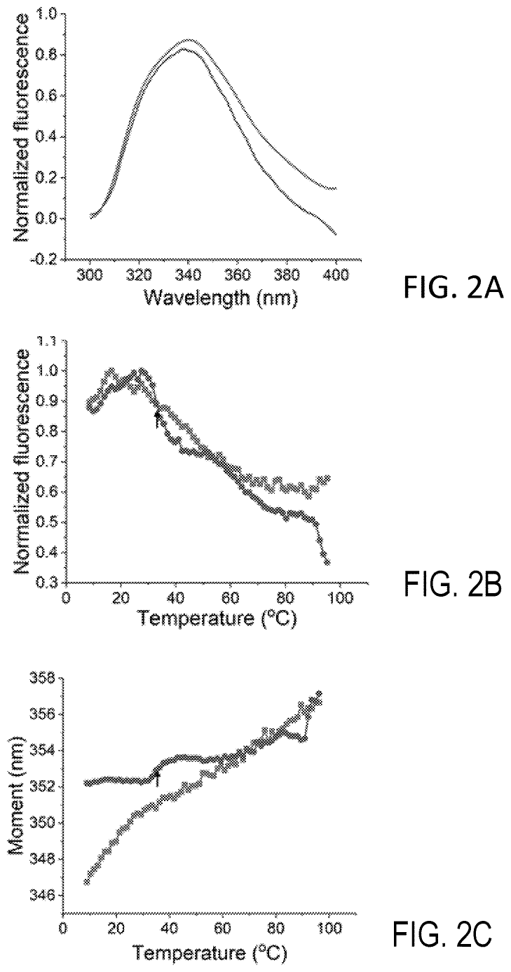

As a second method to assess the L-PaF interaction with ME, we monitored intrinsic (tryptophan) fluorescence of the L-PaF. The intrinsic fluorescence spectra of L-PaF in PBS with and without ME at 10° C. were obtained and normalized so that they could be compared (FIG. 2). PBS and ME alone scans were also obtained so that these background measurements could be subtracted to obtain only the spectral data of the L-PaF. ME by itself showed an unexpected fluorescence signal from 300 to 400 nm when exited at 295 nm, thereby necessitating its subtraction. Whether with or without ME, L-PaF exhibited similar emission spectra with maximal emission around 339 nm at 10° C. This confirmed that the overall tertiary structure of L-PaF was not changed significantly by its association with ME (FIG. 2A). Thermal melting profiles using the normalized fluorescence intensities and moment (MSM peak position) were plotted to compare the thermal transitions for both L-PaF alone and L-PaF with ME (FIG. 2B, 2C). While neither plot of the thermal melts for L-PaF demonstrated an obvious transition (red plots), the L-PaF associated with ME showed a noticeable thermal transition at about 36° C. (indicated by black arrow for each blue plot).

(2) L-PaF Interacts with Mucus-Interacting Chi-C48/80 (Chi)

As a second unique particulate formulation, L-PaF was bound to chitosan (~600 nm particles). Chitosan nanoparticles are highly positively charged under acidic conditions and can interact with negatively charged mucin, which is the major component of mucus layer on the surface of nasal epithelial cells. Indeed, the DLS and zeta potential measurement confirmed the interaction of the preparation of Chi with mucin (Table 2). The Z-average value (size) of Chi increased from 635 nm to 709 nm after adding mucin while the zeta potential was decreased from 17.2 mV to −1.0 mV for Chi-mucin (Table 2). To determine the percentage of L-PaF associated the Chi, we assessed protein adsorption efficiency to Chi by measuring the soluble L-PaF after centrifugation of the binding reaction and found that approximately 35% of the L-PaF was associated with Chi. The relatively low adsorption efficiency can be due to the pI of L-PaF (pI=6.1). We were unable to determine the amount of BECC bound to Chi, however, based on the interaction of Chi with mucin, we assume that the BECC, a negatively charged molecule, is able to interact with the Chi.

TABLE 2
Summary table of particle characteristics
of Chi, mucin and Chi + mucin.

| | | Poly- | Zeta |
|---|---|---|---|
| Name | Z_average | disperity (%) | potential (mV) |
| Chitosan- | 635.40 ± 8.66 | 22.8 ± 5.0 | 17.2 ± 0.36 |
| C48/80 (Chi) | | | |
| Mucin | 370 ± 43.26 | 50.8 ± 8 | −21.7 ± 0.49 |
| Chitosan- | 709 ± 11 | 24 ± 1.2 | −1.0 ± 0.43 |
| C48/80 + mucin | | | |
| (Chi-mucin) | | | |

### (3) L-PaF is Immunogenic Regardless of Formulation

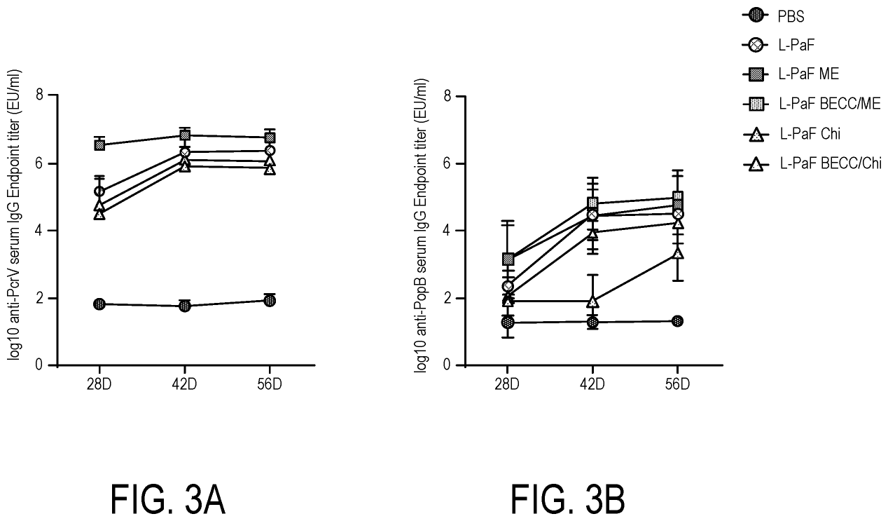

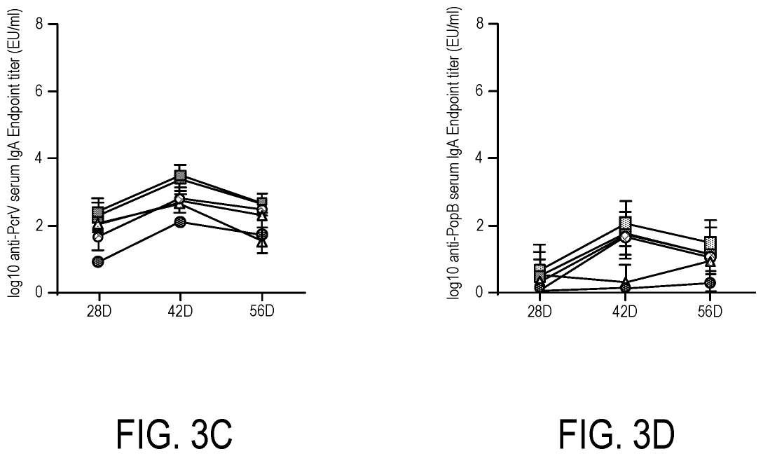

Mice were vaccinated in a prime-boost manner with one prime and two booster doses at 14-day intervals. We found that all formulations containing L-PaF induced comparable amounts of serum antibodies (FIG. 3). While L-PaF BECC/ME elicited the highest antibody responses among all the groups, no statistical significance was detected in most cases (FIG. 3). In contrast, L-PaF Chi at day 42 did not induce comparable antibody levels (for both IgG and IgA) against PopB, while an increase was observed by day 56 (FIG. 3B, 3D). A general trend of increasing antibody production was observed for IgG with the lowest titers at day 28 and similar titers seen at days 42 and 56 against both proteins (FIG. 3A, 3B). In contrast, the highest IgA titers were observed at day 42 with a decline at day 56 (FIG. 3C, 3D).

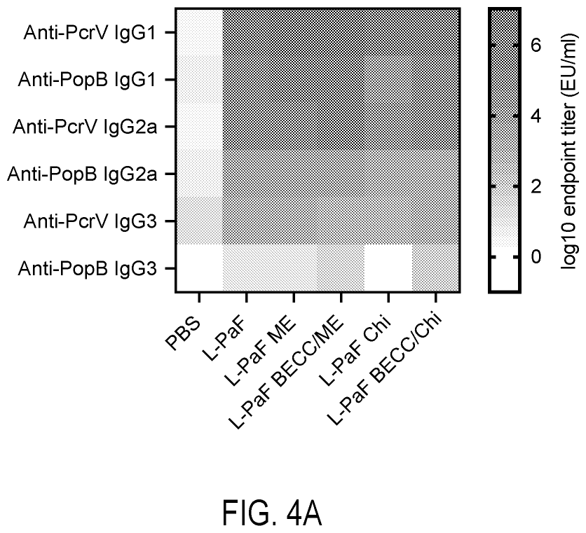

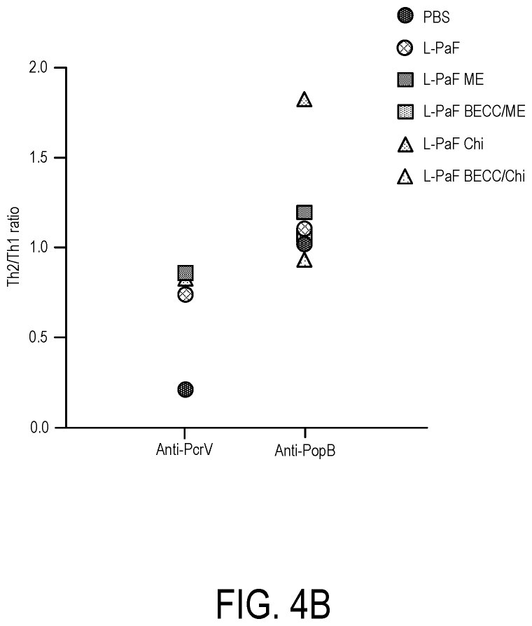

Measurement of different IgG subtypes in day 56 sera (FIG. 4A) showed a slight Th1 bias against PcrV and Th2 bias against PopB (FIG. 4B). The most abundant subtype was found to be IgG1 followed by IgG2a and IgG3. A clear difference was observed in the cases of IgG2a and IgG3, where PcrV caused a greater induction of these antibodies than PopB. The literature indicates that IgG2a and IgG3 (Th1-specific) can fix complement better than IgG1 (Th2-specific). IgG3 can also bind directly to bacteria to provide protection is the same way that IgG1 does. This might have been the cause of the higher OPK response seen previously for PopB vaccinated mice in the absence of complement in vitro (using heat-inactivated serum). Conversely, PcrV appears to impart its potency in vivo by activating the host immune system in a way that leads to complement mediated lysis of Pa. Activation of the adaptive immune response following L-PaF vaccination, thus provides early hints about its effectivity and potency as a Pa vaccine.

### (4) L-PaF Vaccination Leads to a Maintained Th1-Th17 Skewed Cytokine Profile in the Lungs of Immunized Mice Prior to Challenge

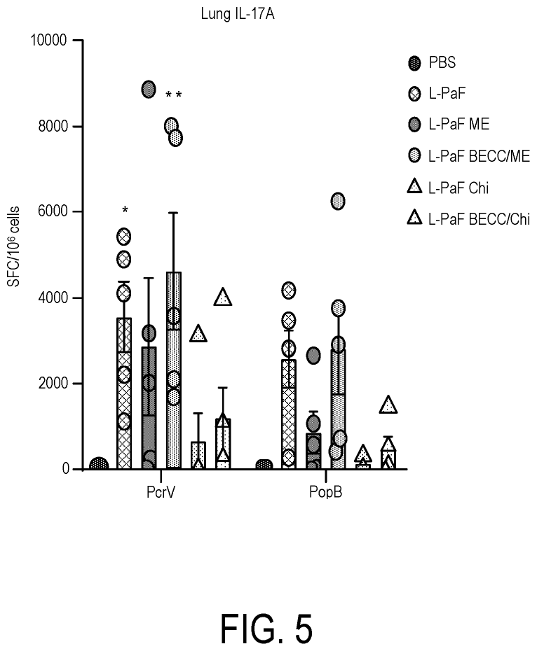

While B cell activation was found to be Th1 skewed, T cell activation followed a Th1-Th17 skewing following vaccination. On day 56, extracted lungs from vaccinated mice (n=5) were processed and IL-17A secreting cells were determined (FIG. 5). Cells from mice vaccinated with L-PaF alone or with BECC/ME that were treated with PcrV had significantly higher frequencies of IL-17A secreting cells compared to the control groups. While IL-17A secreting cells were also found after treatment with PopB, no statistical significance was observed. Technical negative control cells (not receiving any protein stimulation) did not show any significant IL-17A secreting cells either.

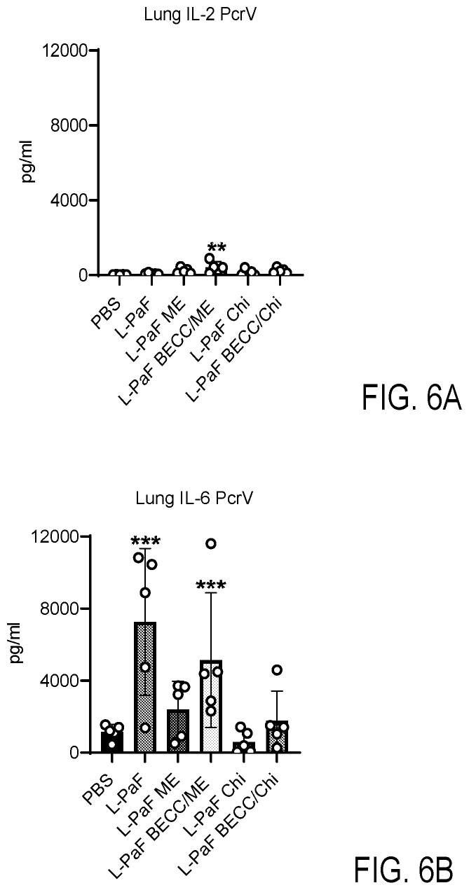

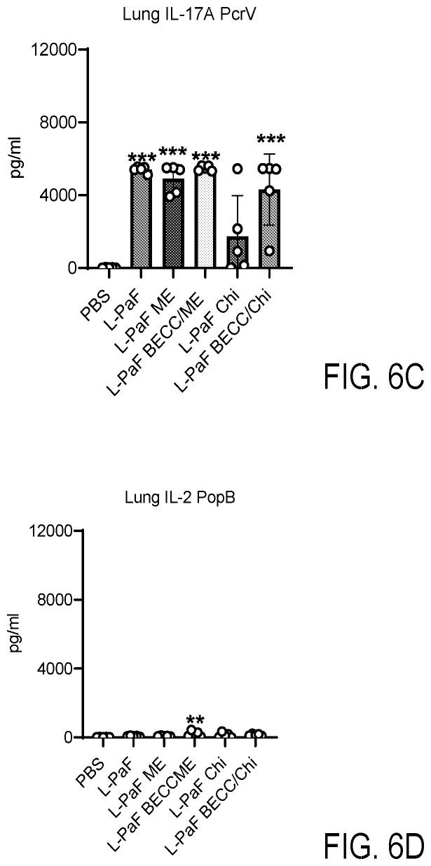

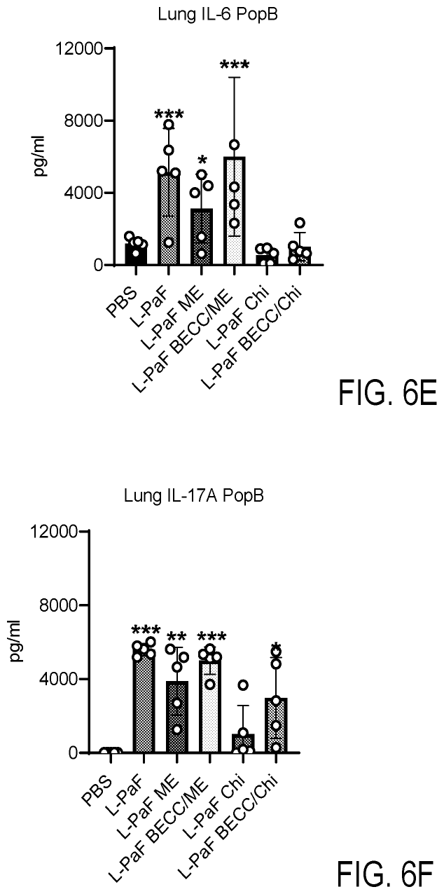

We also used pre-challenge lung cells to assess the secretion of different cytokines after stimulation with PcrV or PopB. Th1-Th17 bias was measured based on detection of potent Th1 and Th17 cytokines, namely, IFN-γ, the master regulator IL-2, pro-inflammatory IL-6, TNF-α and IL-17A. Although no statistical significance was found for IFN-γ and TNF-α, the other three cytokines showed different secretion patterns among the groups. (FIG. 6). IL-2, IL-6 and IL-17A were significantly upregulated in lung cells from the L-PaF BECC/ME immunized mice (after stimulation with either PcrV or PopB). IL-2 is the master regulator that activates various downstream pathways leading to immune response. Significant upregulation was seen for PcrV and PopB treated cells collected from L-PaF BECC/ME immunized mice (FIG. 6 A,D). PcrV and PopB treated cells collected from L-PaF and L-PaF BECC/ME immunized mice, respectively, also had elevated amounts of IL-6 (FIG. 6 B,E). Conversely, significant secretion of IL-17A was detected from cells from all the immunized mice groups except for L-PaF Chi (FIG. 6 C,F). Cells from mice vaccinated with PBS had very low or no response under any treatment conditions. Technical control group, where no protein stimulation was provided, only had significant IL-17A in L-PaF BECC/ME immunized lung cells (<500 pg/ml), but this was much lower than that seen for the treated groups (>3000 pg/ml). The ability to generate IL-17A without any treatment indicates the immunogen's potency towards protection.

### (5) Protective Efficacy:

### (a) the In Vivo Lung Burden was Reduced Significantly for the ME- and ME BECC-Containing Groups

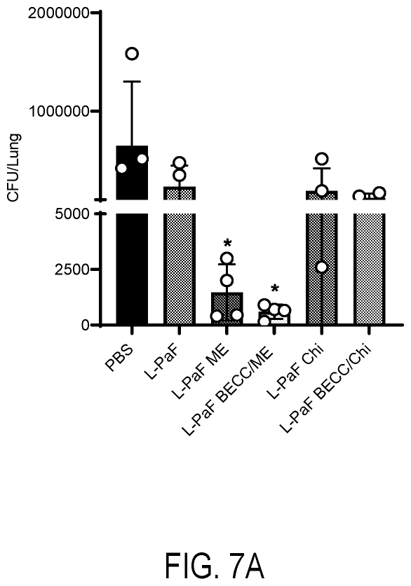

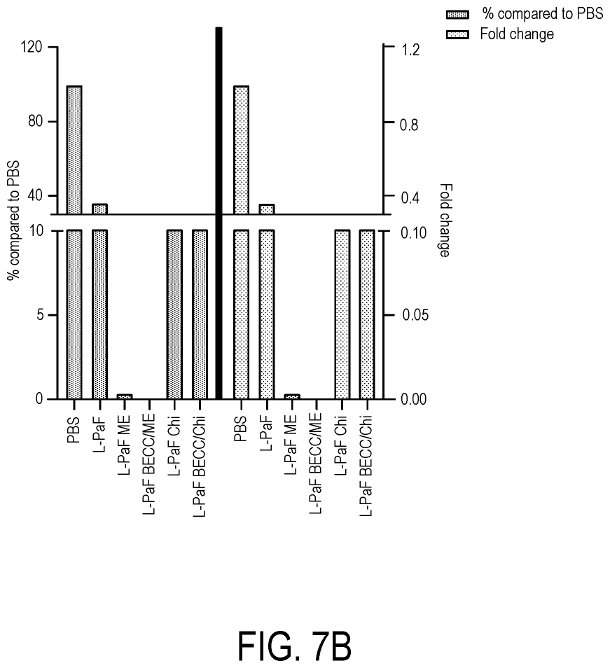

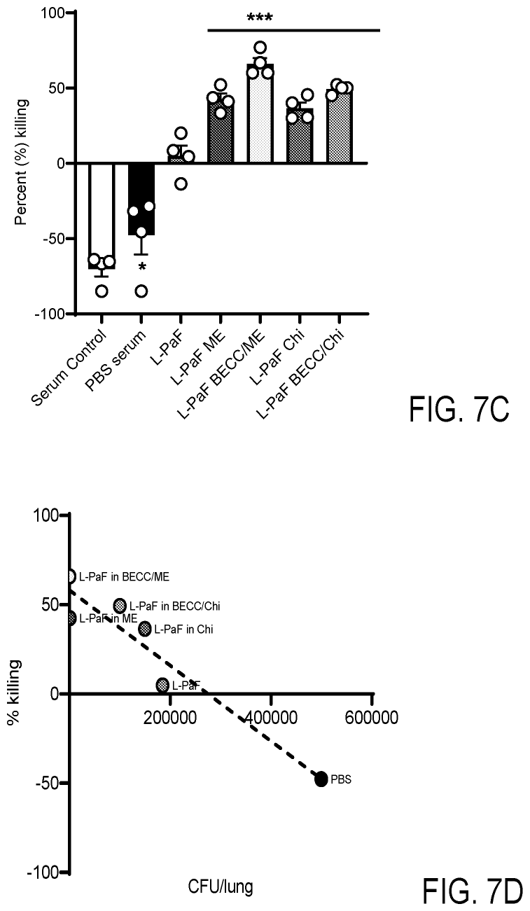

On day 56, we challenged the vaccinated mice with the clinical mucoid isolate Pa mPa08-31 and monitored their morbidity over a 3-day period. No mortality was seen during this time. While PBS mice were sick with higher health scores, indicating morbidity, the immunized mice displayed little or no morbidity. Extracted lungs were processed and CFU lung burden was determined by dilution plating of the homogenates on Pseudomonas Isolation Agar (PIA) (FIG. 7A). Lungs from L-PaF ME and L-PaF BECC/ME vaccinated mice had significantly lower bacterial counts than the other groups. Lung bacterial burden is a clear indicator of the immunogen's protective efficacy in mice, with the highest fold change (and % compared to PBS) observed for the L-PaF BECC/ME vaccinated group (FIG. 7B).

(b) Serum from the Immunized Groups Reduced Bacterial Burden In Vitro

Opsonophagocytic killing (OPK) is an important marker of in vitro functional protective efficacy. Sera from mice immunized with all L-PaF formulations were found to possess significant bactericidal capability (FIG. 7C). Serum from immunized groups showed a bacterial killing ability in the range of 4-76%, with highest killing potential seen for sera from mice vaccinated with L-PaF BECC/ME. Although L-PaF BECC/Chi group showed a slightly higher killing ability than L-PaF ME in vitro, this was at odds with protective capacity in vivo. A negative percent killing translates into bacterial replication and this was only seen for the serum control and PBS groups. Although L-PaF alone serum did not possess a striking level of OPK ability, it was significantly better that the controls in which the Pa population expanded. When the in vivo and in vitro data were compared, a clear negative correlation was found, meaning that a higher in vitro killing (OPK) coincides with a reduced bacterial burden in the lung (FIG. 7D). These results demonstrate the immunogenicity and protective efficacy of L-PaF with or without formulation, but they do not illustrate the mechanism of protection.

(c) Colonization Reduction and Lesser Morbidity Associates with Post-Challenge In Vivo High IL-17A and Low TNF-α

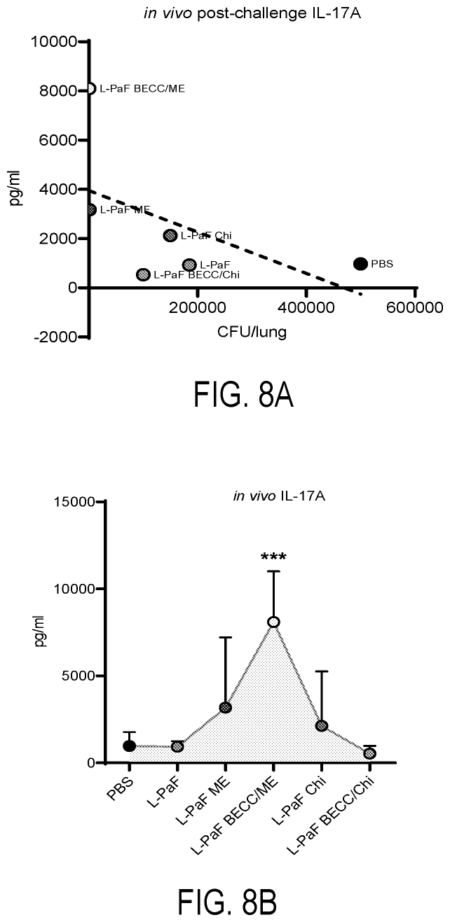

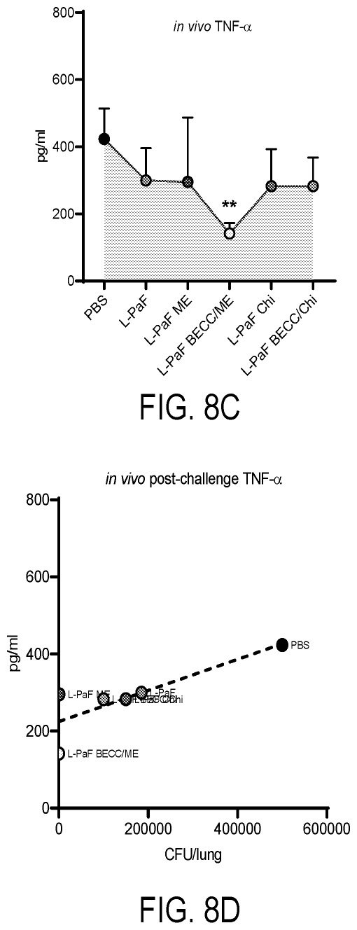

To begin dissecting the mechanism of protection elicited by the best L-PaF formulation(s), we further assessed lung cells from the challenged mice to detect secretion of pro-inflammatory cytokines, IL-17A and TNF-α, which play a crucial role following immunization and challenge (FIG. 8). Unlike the pre-challenge cells, these cells were not stimulated with PcrV or PopB prior to cytokine measurement. Thus, cytokines here were generated because of the infection/challenge regardless of whether the mice were vaccinated. The L-PaF BECC/ME group showed significantly higher levels of IL-17A secretion than the other groups (FIG. 8A). This was inversely correlated with lung burden: i.e., less IL-17A produced post-challenge equates to a higher lung burden (FIG. 8B). This same group had significantly less TNF-α in comparison to the other groups (FIG. 8C). The literature indicates that IL-17A is an important cytokine for proper immune stimulation and maintenance against Pa infection. That was the case for this study as well with high IL-17A in pre-challenge lung cells (FIGS. 5 & 6). Furthermore, another trend that was observed here was that a sustained presence of pro-inflammatory cytokines (IL-2, IL-6, IL-17A) prior to infection deemed important to reduce the extent of the infection. On the other hand, a high TNF-α following Pa challenge was associated with high bacterial burden (FIG. 8D). As already described, difference in expression of pre-challenge TNF-α was not statistically significant among the groups. This sudden onset of TNF-α in the groups except for L-PaF BECC/ME, imbalances the host immune system, leading to a break in the homogeneity. Therefore, high post-challenge in vivo IL-17A acted as a protective barrier against infection just like the pre-challenge levels, while a high TNF-α following infection was positively associated with lung burden.

### b) Conclusion

This work provides an improved understanding towards biophysical and immunological characteristics of L-PaF (LTA1-PaF). This fusion protein was tested in light of two different formulations, namely, the oil-in-water emulsion ME and the chitosan nanoparticle Chitosan-C48/80 (Chi). The further contribution of the TLR4 agonist, BECC, was also monitored. Comparative analysis of these newly formulated immunogens showed that the L-PaF BECC/ME formulation was highly immunogenic and provided the best protective efficacy. The observed Th1/Th17 skewed immune response resulted in upregulation of different pro-inflammatory cytokines pre-challenge, further contributing to protection. On the other hand, the presence of elevated TNF-α post-challenge was found to directly correlate with higher bacterial burden in mice lung. Because of the nature of the L-PaF BECC/ME emulsion (size, composition, and surface biophysical properties), it is possible that this novel formulation is better suited for use in humans than is the L-PaF alone, which is a simple subunit vaccine.

### c) Material and Methods

### (1) Materials

Squalene was from Echelon Biosciences (Salt Lake City, UT), Chitosan and C48/80 were from Millipore-Sigma (St. Louis, MO). All other buffers chemicals were reagent grade.

### (2) Protein Preparation

L-PaF was made. Briefly, E. coli Tuner cells expressing L-PaF/His-Tag PcrH were grown in TB media supplemented with chloramphenicol (34 μg/ml) with a fed-batch mode in a 10 L bioreactor (Labfors 5, Infors USA Inc., MD). An overnight starter was expanded to 1 L and ~800 mL was transferred to the bioreactor containing 9 L of TB media supplemented with chloramphenicol (34 μg/ml). The culture temperature was maintained at 30° C. and protein expression was induced adding IPTG to 1 mM when the culture reached an A₆₀₀ of ~25. After 3 h, the bacteria were collected and processed for purification. The L-PaF/His-Tag PcrH was captured on an IMAC column followed by Q anion exchange chromatography. Lauryldimethylamine oxide (LDAO) was added to a final concentration of 0.10% to release the HT-PcrH. The protein solution was passed over a final IMAC column with the L-PaF passing through the column. L-PaF was dialyzed into PBS with 0.05% LDAO and stored at −80° C. LPS levels were determined using a NexGen PTS with EndoSafe cartridges (Charles River Laboratories, Wilmington, MA). All proteins had LPS levels <5 Endotoxin units/mg protein based on anlaysis using an Endosafe system (Charles River Labs).

### (3) Preparation of L-PaF ME and L-PaF BECC/ME Formulations

Squalene (8% by weight) and polysorbate 80 (2% by weight) were mixed to achieve a homogenous oil phase. Using a Silverson L5M-A standard high-speed mixer, 40 mM Histidine (pH 6) and 20% sucrose were added to the oil phase and mixed at 7500 RPM followed by six passes in a Microfluidics 110P microfluidizer at 20,000 psi to generate a milky emulsion of 4XME (MedImmune Emulsion). Polysorbate 80 acted as an emulsifying agent to stabilize the emulsion. BECC (2 mg/ml) was prepared in 0.5% triethanolamine by vortexing followed by sonicating for 30 min in a 60° C. water bath sonicator until the BECC was completely dissolved. The pH of BECC solution was adjusted to 7.2 with 1 M HCl. To make the L-PaF with ME, the protein was added to the ME with a final concentration of 0.67 mg/ml, vortexed and allowed to incubate overnight at 4° C. To make the L-PaF with ME and BECC formulation, ME and BECC were mixed by vortexing for 2 min and incubated overnight at 4° C. The next day, L-PaF was mixed with ME-BECC solution at a volumetric ratio of 1:1 to achieve desired final antigen concentration.

### (4) Preparation of L-PaF Chitosan-C48/80 (Chi) and L-PaF BECC/Chi Formulations

To make chitosan nanoparticles, 1 gm of chitosan was added in 10 mL of a 1 M NaOH and stirred for 3 h at 50° C. The chitosan solution was then filtered through 0.45 μm membrane and the resulting pellet was washed with 20 mL of MilliQ water. The recovered chitosan was resuspended in 200 mL of 1% (v/v) acetic acid solution and stirred for 1 hour. The solution was filtered through 0.45 μm membrane, and 1 M NaOH was added to adjust the pH to 8.0, resulting in purified chitosan. Purified chitosan was vacuum dried for 24 hours at 40° C. C48/80 loaded chitosan nanoparticles (Chi) were prepared by adding dropwise 3 ml of an alkaline solution (5 mM NaOH) containing C48/80 and Na2SO4 (0.3 mg/mL and 2.03 mg/mL, respectively) to 3 ml of a chitosan solution (1 mg/ml in acetic acid 0.1%) with high-speed vortexing. The Chi was formed using magnetic stirring for an additional 1 h. Chi was then collected by centrifugation at 4500×g for 30 min and the pellet resuspended in MOPS buffer (20 mM, pH 7). The L-PaF in PBS was also exchanged into MOPS buffer (20 mM, pH 7) using an Amicon Ultra-4 centrifugal filter. To make L-PaF Chi, L-PaF was added to the Chi solution to a weight ratio of 1:4. To make L-PaF BECC/Chi, the nanoparticles were mixed with BECC by vortexing and incubating for 10 min. L-PaF was then added, mixed by vortexing and incubated for 2 h at 4° C.

### (5) Intrinsic Tryptophan Fluorescence

Intrinsic tryptophan fluorescence spectra were obtained. Briefly, intrinsic tryptophan fluorescence was measured by a fluorescence plate-reader (Fluorescence Innovations, Minneapolis, MN), which is equipped with a tunable pulsed dye laser, a temperature controlled 384-well sample holder (Torrey Pines Scientific, Carlsbad, CA), and a high-speed digitizer. L-PaF and formulated L-PaF samples (20 μl) were loaded into a Hard-Shell 384-well PCR plates. Samples were excited at 295 nm and steady state emission spectra were collected using a charged coupled device detector from 310 nm to 400 nm. Fluorescence moment (mean center of spectra mass peak position or MSM peak position) was reported. Temperature ramps were set from 10 to 95° C. with an increment of 1° C. per step and an equilibration time of 60 sec at each temperature. Moment (MSM peak position) were plotted as a function of temperature and first derivative of the resulting data was used to calculate the melting temperature (Tm) using Origin 7.0 (OriginLab, Northampton, MA).

### (6) Size and Zeta Potential

The hydrodynamic diameter of L-PaF and formulations were determined using dynamic light scattering (DLS) with Zetasizer Ultra (Malvern Instruments). Formulations were diluted in 1:10 with water in triplicate and measured in disposable polystyrene cuvettes. The SOP parameters were set up as following: material RI=1.59, dispersant RI (water)=1.33, T=25° C., viscosity (water)=0.887 cP, measurement angle=173° backscatter with automatic attenuation. The Z-average values of the hydrodynamic diameter of samples were calculated via cumulant analysis. To gain more information on the particle size and concentration, Multi-Angle Dynamic Light Scattering (MADLS) measurements with the Zetasizer Ultra were performed to collect the intensity of backscattering, forward scattering, and side scattering.

Zeta potential measurements were performed via electrophoretic light scattering using the same instrument as that used to measure particle size. Samples were diluted 10-fold in water before analysis. Samples were introduced into disposable folded capillary cells at 25° C. Scans were performed until the results had acceptable correlation functions (typically 50-100 scans). Three independent measurements were performed, and the zeta potential was calculated based on electrophoretic mobility of sample particles.

### (7) Mice and Immunizations

The mouse animal protocols were reviewed and approved by the University of Kansas Institutional Animal Care and Use Committee Practices (protocol AUS 222-03). Six- to eight-week-old BALB/c mice (n=10) (Charles River Laboratories, Wilmington, MA) were used for all experiments. Prior to administration, the following were prepared in 30 μl volumes: PBS, 1 μg L-PaF (L-PaF), 1 μg L-PaF in ME (L-PaF ME), 1 μg L-PaF in BECC-ME (L-PaF BECC/ME), 1 μg L-PaF in Chitosan-C48/80 (L-PaF Chi), and 1 μg L-PaF in BECC-Chitosan-C48/80 (L-PaF BECC/Chi). For immunizations, mice were anesthetized using isoflurane and vaccine formulations administered intranasally (IN). Immunizations were on days 0, 14 and 28 for this study. Blood was collected prior to each vaccination and at days 42 and 56.

### (8) Antigen-Specific IgG and IgA

Antibodies specific for PcrV and PopB were determined by ELISA. Briefly, 96-well plates coated with PcrV or PopB (1 μg/mL in PBS) were blocked overnight with 10% milk (Omniblok, americanbio) in PBS. Each well was incubated with serum samples for 1 h at 37° C. After washing the plates with PBS-Tween (0.05%), secondary antibody (KPL, Gaithesburg, MD) was added and incubated for 1 h at 37° C. Levels of IgG (H+L) and IgA were determined using horseradish peroxidase-conjugated secondary antibodies (human serum adsorbed) raised in goat (Southern Biotech, Birmingham, AL). 3,3′,5,5′-Tetramethylbenzidine (TMB) substrate was added, and reaction was stopped with H₃PO₄. Endpoint titers were calculated and represented as ELISA units per ml (EU ml⁻¹).

### (9) Opsonophagocytosis (OPK) Assay

OPK was carried out. Briefly, Pa strain mPA08-31 was grown overnight. A new culture was started by adding 200 μl of the overnight culture to 20 ml of LB media and grown to A₆₀₀ of 0.3. Bacteria were collected by centrifugation and a portion of the resuspension adjusted to a concentration of 2×10⁷ cells/ml in Minimal Essential Medium (MEM, ThermoFisher, Waltham, MA) containing 10% bovine serum albumin (BSA, Sigma, St. Louis, MO). The J774A.1 (ATCC, Manassas, VA) murine macrophage cell line was grown to 90% confluency in Dulbecco's Modified Eagle's Medium (DMEM, ThermoFisher, Waltham, MA) and was adjusted to allow for a final multiplicity of infection (MOI) of 0.1 in 10% MEM-BSA. At 42 Days Post-Immunization (DPIm) sera from the vaccinated mouse groups (5 μl from each mouse were mixed) were heat treated at 56° C. to destroy complement. Serum (1:500), bacteria and macrophages were mixed at a 1:1:1 ratio to a final volume of 300 μl and kept for 30 min at 37° C. The suspension was then serially diluted and plated on Pseudomonas Isolation Agar (PIA, BD, Franklin Lakes, NJ). Percent killing=[((CFU from T₀)−(CFU from T30 min))/(CFU from T₀)×100]. An appropriate serum control was used where MEM-BSA was used in the published protocol. Technical quadruplets from each group were assessed and statistical comparisons made as described below.

### (10) IL-17A ELISpot

Immunized mouse lungs were extracted and processed to single cell suspensions according to manufactures specifications (Miltenyibiotec). Lung cells (1×10⁶ cells/well) were incubated for 24 h at 37° C. in the presence of 5 μg/ml PcrV, PopB or PBS, in plates coated with antibodies against IL-17A for a color assay as per manufacturer's specifications (ImmunoSpot). The IL-17A secreting cells were quantified using a CTL immunospot reader. Biological negative controls were maintained as PBS mice group while technical negative controls were cells without any treatment.

### (11) Cytokine Determinations

Lung cells were incubated with 10 μg/ml PcrV, PopB or PBS for 48 h at 37° C. Supernatants were collected and analyzed with U-PLEX kits for cytokines: IFN-γ, IL-2, IL-6, IL-17A and TNF-α. Cytokine concentrations were determined using an MSD plate reader with associated analytical software (Meso Scale Discovery, Rockville, MD). For post-challenge cytokine determination, cells were not stimulated with PcrV or PopB. Instead, mice lungs were assessed to determine in vivo pro-inflammation resulting from bacterial challenge. Control groups were maintained as described above.

### (12) Pseudomonas aeruginosa Challenge

The mucoid Pa strain mPA08-31 was streaked onto Pseudomonas isolation agar (PIA) and incubated overnight at 37° C. with shaking at 180 rpm. A 200 μl aliquot from the overnight culture was inoculated into 20 ml of LB and grown at 37° C. with 250 rpm shaking the A_{600 nm} reached ~0.3. The Pa were collected by centrifugation, washed once, resuspended in PBS, and diluted to 4×10⁷ CFU/30 μl. On day 56, mice were anesthetized by isoflurane and challenged IN. On day 3 post-infection, mice (n=4) from each group were euthanized and the lungs were collected and processed to assess the immune response in terms of secreted cytokine levels Additionally, the CFU/lung was enumerated for each mouse by plating a portion of the lung extract on PIA.

### (13) Correlation Studies

Linear correlation studies were carried out using bivariate correlation in the form of Pearson r. They were further analyzed via simple linear regression. Both operations were carried out with 95% confidence interval, using GraphPad Prism 8.1.2.

### (a) Cytokines and Lung Burden.

Post-challenge lung cytokines and lung burden were checked for linear correlation as described above.

### (b) OPK and Lung Burden

in vitro bacterial killing ability of the serum was checked with in vivo lung burden to find out a possible correlation between them.

### (14) Statistical Analysis

GraphPad Prism 8.1.2 was used to generate graphics and to perform all statistical comparisons. Differences among treatment groups were analyzed using two-way ANOVA. Challenge groups were compared to PBS with Dunnett's multiple comparisons tests. A p value of less than 0.05 was considered significant for all comparisons. * P<0.05; ** P<0.01; *** P<0.001. Pearson's r values were mentioned at appropriate places along with R squared values from simple linear regression analyses.

### 2. Example 2: a Broadly Protective Prophylactic Vaccine Against Pseudomonas aeruginosa

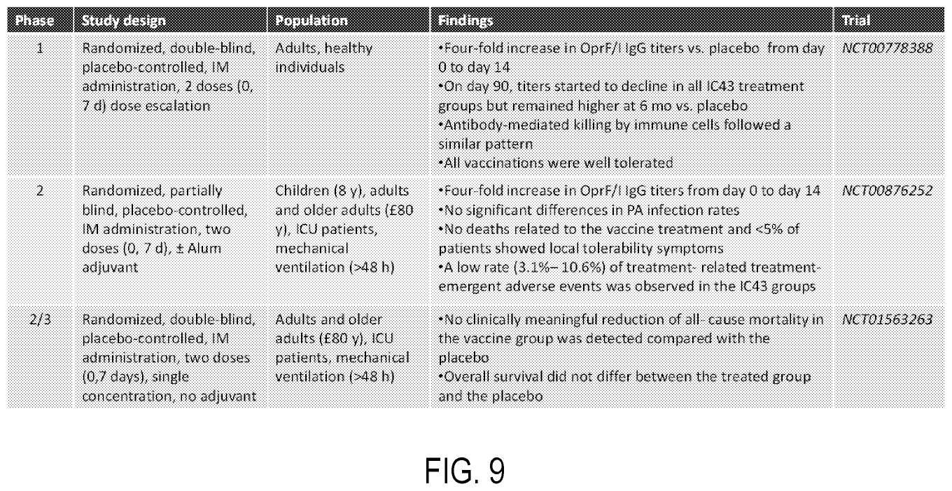

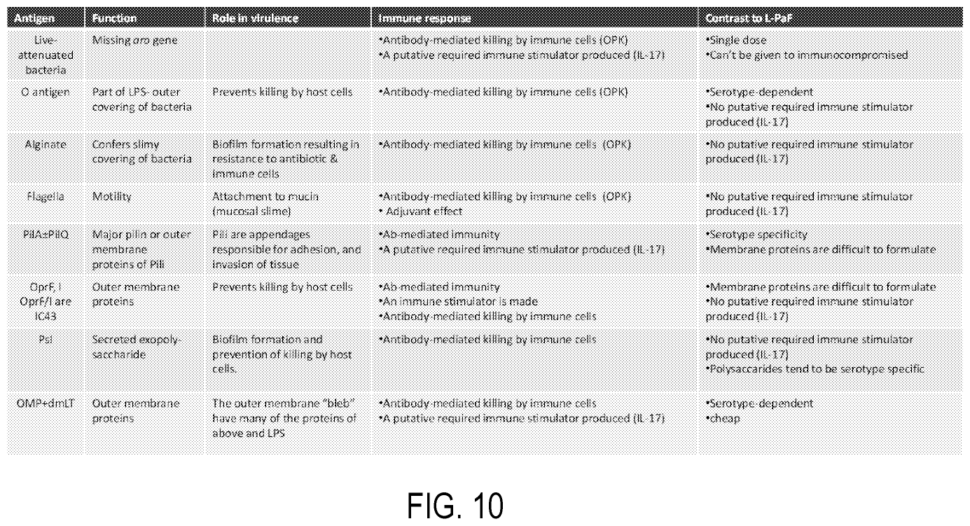

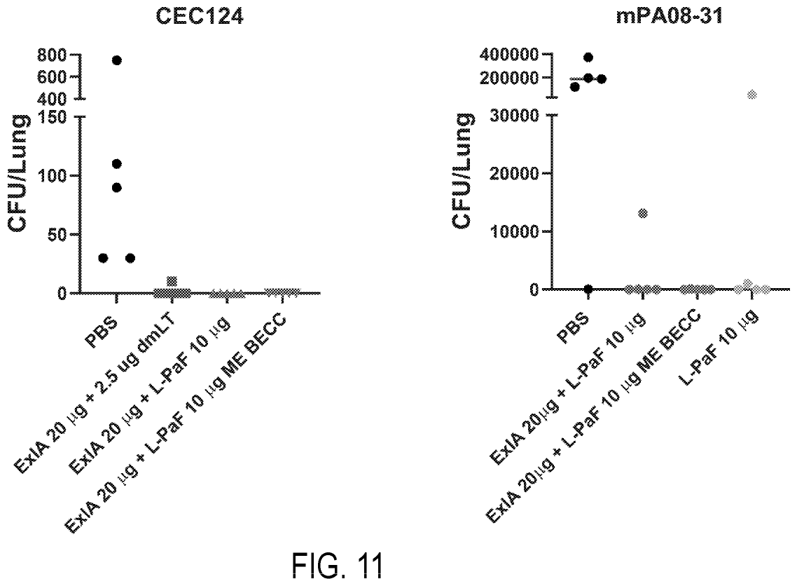

Vaccination is perhaps the greatest public health achievement. There are no licensed vaccines to prevent Pa infections, however, several are in the pipeline. Here we consider this task in light of other vaccine candidates (FIGS. 9 and 10), which can largely be separated into distinct groups: 1) whole killed or live-attenuated; 2) outer membrane (OM) proteins/LPS; 3) specific surface proteins of the flagella, pili or the type III secretion system (T3SS); 4) or a combination of these. While the first group could give the best protective immune response, it suffers from potential reactogenicity and other risks when administrated to immunocompromised individuals. The second group including OMVs can produce a serotype-specific response with the outer membrane proteins being either difficult to purify or prepared as only the surface exposed portion of the antigen. These remain viable candidates since the surface exposed proteins are often responsible for attachment and are critical for pathogenesis onset. A disadvantage of flagellin and pili is that they, too, can led to serotype specificity. The advantage of the surface proteins of the T3SS, PcrV and PopB, is that they are highly conserved with mutations resulting in loss of virulence. The exception to this are strains of the PA7-like clade that lack the T3SS and instead lyse cells via exolysin A (ExlA), ShlA or FhaC. Group 4 is a combination of the key protective vaccine groups. The vaccine disclosed herein falls into group 4 with the use of PcrV, PopB and ExlA.

PaF is a fusion of two essential surface localized T3SS proteins, PcrV and PopB, which are >96-99% conserved among PAO1/PA14-like strains. Because these are T3SS scaffold proteins required for the early stages of pathogenesis for PAO1/PA14-like strains, vaccine escape is unlikely since mutation of these proteins impacts assembly of the T3SS apparatus, rendering the mutant non-pathogenic. The LTA1 subunit from the labile toxin (LT) of Enterotoxigenic E. coli serves as the adjuvant. LTA1 is genetically fused to PaF (L-PaF) to allow simultaneous uptake of adjuvant-antigen by antigen presenting cells to enhance cellular immunity. Unlike dmLT (double-mutant LT), LTA1 retains the toxin's ADP-ribosylation (ADPr) activity and the ability to promote dendritic cell (DC) maturation, but does not possess detectable toxicity. LTA1 stimulates a balanced Th1/Th2 response along with a mucosal response characterized by production of mucosal IgA, as well as IL-17. Recently, it was shown that the addition of LTA1 to Fluzone® increased IgA, while decreasing levels of IL-6 post-H1N1 challenge.

While the T3SS has been studied for years, including that of Pa, it has recently been discovered that it is absent in the so called outlier strains of the PA07 clade. Rather than a T3SS, these strains use exolysin A (ExlA) to disrupt host cell membranes. ExlA is part of the ExlAB two-partner secretion system. The ExlB forms a protein channel in the outer membrane. The ExlA is then secreted into the periplasm where it is cleaved to a form that is translocated across the outer membrane via the ExlB. ExlA then interacts with the host cell membrane via a required linkage with the type IV pili of the Pa. This interaction results in disruption of the host cell. It should be noted that ExlA absent the type IV pili cannot disrupt host cell membranes. We have included the C-terminal portion of ExlA (the lytic portion) with L-PaF in the vaccine since omission of the ExlA promotes “escape” of the PA07 strains and allow these strains to expand unimpeded in an L-PaF vaccinated world. The disclosure herein describes the only vaccine that can make such a claim. It is understood and herein contemplated that exolysin A is not the only protein that can be used to disrupt the cell membrane. Other pore-forming toxins can also be used, including, but not limited to the pore forming toxin ShlA of the two partner secretion system ShlBA from Serratia marcescens; or the pore forming protein FhaC of the two partner secretion system FhaBC from Bordetella pertussis. In some aspects, the ExlA, ShlA, or FhaC can further be modified to also comprise LTA1.

Live-attenuated or killed vaccines are naturally immunogenic with inherent adjuvants and protein multimers. This is not the case for protein subunit vaccines and is postulated to be responsible for their lack of efficacy. They do not elicit the danger signal to trigger the immune system. As such, they must be formulated with the danger signals. Thus, we produce a bacteria-like particle. ExlA and PaF of L-PaF are the broadly protective antigens. LTA1 of L-PaF provides an adjuvant that stimulates a balanced Th1/Th2 response along with a mucosal response characterized by production of mucosal IgA, as well as IL-17. In addition, the TLR4 agonist, BECC438 (bacterial enzymatic combinatorial chemistry adjuvant #438) is formulated into the particle. BECC438 has been documented to increase humoral and cellular immune responses to elicit a balanced Th1/Th2 response that provides protection against bacterial pathogens such as the extracellular pathogen Yersinia pestis. The rationale of using BECC438 is that it can enhance the production of antibodies required for opsonophagocytosis, which contributes to Pa clearance. Finally, in this formulation, we produce an oil-in-water containing L-PaF, ExlA and BECC, which produces a 100 nm droplet that elicits the danger signal. The L-PaF/ExlA/BECC-emulsion formulation provides a complete Pa vaccine to cover all Pa strains that can be safe for all and that is defined. This formulation is delivered intranasally to elicit the mucosal response in the lungs, which is a prime target for Pa infections. It can be delivered intramuscularly.

The preferred formulation is L-PaF+ExlA+BECC438 in an oil and water emulsion we call ME, which contains 20 mM Histidine, pH 6, 10% sucrose, 4% Squalene, 1% Polysorbate-80. Ten female Balb/C mice were vaccinated with PBS, 20 μg ExlA+10 μg L-PaF, or 20 μg ExlA+10 μg L-PaF+ME+10 μg BECC, three times on days 0, 14, 28. Additionally, five mice were vaccinated with 20 μg ExlA+2.5 μg dmLT (double-mutant labile toxin where the active moiety is LTA1) (positive control for the ExlA+Pa strain) and five mice were vaccinated with 10 μg L-PaF (positive control for the T3SS+ strain). On day 56, mice were challenged with either CEC124 or mPA08-31 (FIG. 11). CEC124 is of the PA07 clade and expressed ExlA and is devoid of the T3SS (PcrV-PopB−). mPA08-31 is of the PA01/PA14 clade and expresses the T3SS (PcrV+PopB+) and is devoid of ExlA. For those mouse groups challenged with the CEC 124, the group vaccinated with PBS had recoverable Pa while those vaccinated with ExlA in the formulation were essentially cleared of the Pa (FIG. 11, left panel). Similarly, for those mouse groups challenged with the mPA08-31, the group vaccinated with PBS had recoverable Pa while those vaccinated with L-PaF in the formulation were essentially cleared of the Pa (FIG. 11, right panel).

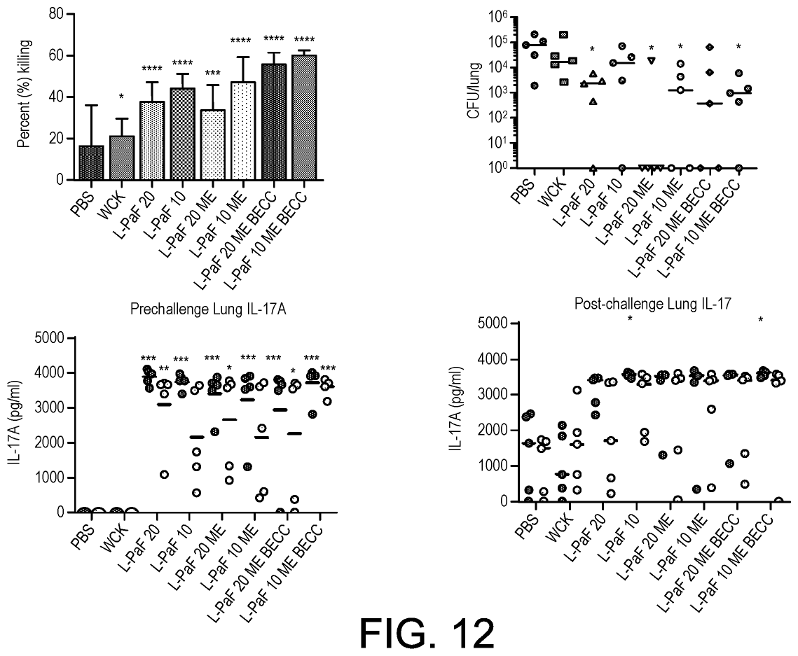

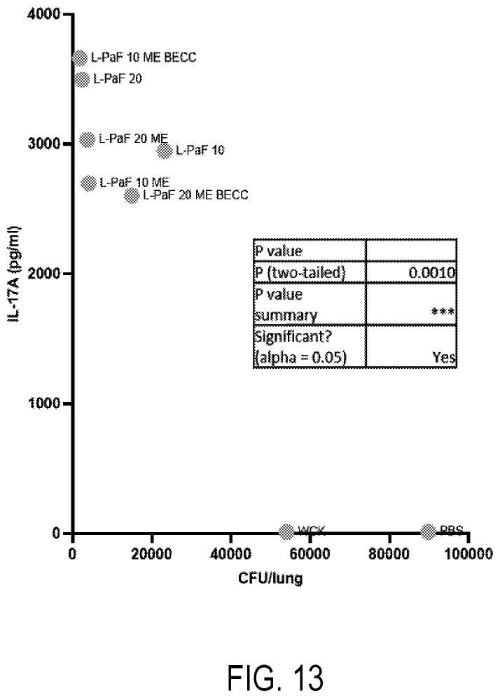

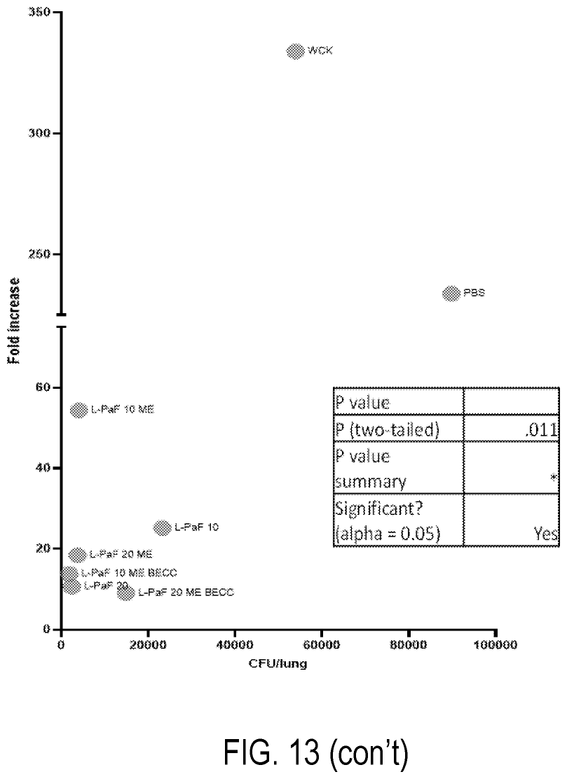

It has been postulated that a successful human vaccine can exhibit high opsonophagocytosis activity (OPK) and elicit high levels of IL-17. The L-PaF+BECC438 in ME elicited these activities. In another set of experiments with L-PaF+BECC438+ME using two doses of L-PaF, significantly higher OPK activity was seen in the L-PaF groups than PBS, but no significant differences between the L-PaF groups. However, the trend is an increase in the OPK activity after the addition of the ME and BECC. (FIG. 12, top left). These trends were translated into protection with the 10 L-PaF+BECC+ME eliciting the lower CFU/lung burden (FIG. 12, top right). Lung cells from pre-challenged mice and from the mice used to determine CFU/lung were stimulated with PopB or PcrV and the levels of secreted IL-17A, determined. All pre-challenge PBS and WCK groups have either no or little cytokine secreted while the L-PaF groups are higher with all L-PaF groups stimulated with PopB secreting significant levels of IL-17 (FIG. 12, bottom left). In contrast, the secreted cytokine levels post-challenge are higher for the PBS and WCK groups while the L-PaF groups remaining at the same levels. (FIG. 12, bottom right). Additionally, the IL-17A levels correlate with protection (FIG. 13, top left).

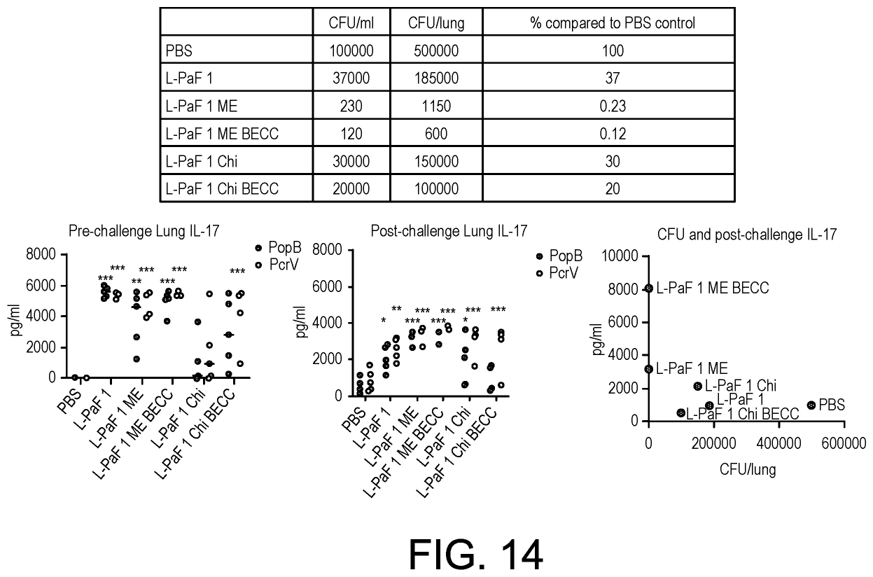

While the bacterial-like particle of the above formulation was produced via the emulsion ME, we have also used the nanoparticle chitosan. The formulation (chi) consisted of 0.15 mg/mL C48/80, 1.02 mg/mL Na2SO4, 0.5 mg/mL chitosan. Mice were vaccinated as above with PBS, WCK (a whole Pa cell, killed formulation), 1 μg L-PaF alone or with ME, ME+BECC, Chi, or Chi+BECC) (FIG. 14). When mice were challenged with mPA08-31, the L-PaF formulated in the ME+BECC provided the best protection, but the Chi and Chi+BECC did reduce the CFU/lung as compared to the PBS mice (FIG. 14, top panel). When the post-challenged lung cells were stimulated with PcrV and PopB, the correlation with protection was better with the ME formulation, however, the correlation with protection for the Chi formulations were better than the PBS vaccinated mice.

While these formulations have been delivered IN to naïve mice, these formulations can also be effective intramuscularly (IM). Additionally, while cystic fibrosis children can be vaccinated as soon as diagnosed and likely be naïve to Pa at that time, older humans would not be naïve. Thus, older adults can be vaccinated IM having already triggered the mucosal arm of the immune system. These experiments are ongoing. Additionally, BECC438 is a biosimilar to MPLA and PHAD. It is envisioned that these TLR4 agonists can be used, if available. Experiments with PHAD can determine whether BECC438 is superior. Lastly, the ME formulation contains excipients as well as squalene. Experiments are underway to examine the need to squalene or whether another oil such as soybean oil can be used.

With respect to animal models, the current results have been collected from mice. We are in the process of examining the efficacy in the cystic fibrosis rat model and in aged mice. We have determined that the L-PaF+BECC can increase the OPK activity and reduce the Pa burden in wild type rats.

We have defined a current formulation of L-PaF+ExlA+BECC in the ME oil-in-water emulsion delivered intranasally in young Balb/C mice. The L-PaF and ExlA are required for this vaccine to be broadly protective. Additionally, the final formulation must increase the OPK activity, IL-17 levels and reduce the Pa burden in mice/rats. Other routes, formulations, aged or immunocompromised mice/rats are in progress. While the formulations have been delivered IN to naïve mice, that these formulations can be effective intramuscularly (IM) to either naïve or Pa pre-exposed mice/rats. For example, while cystic fibrosis children can be vaccinated as soon as diagnosed and likely be naïve to Pa at that time, adolescent and older humans would not be naïve. Thus, these populations can be vaccinated IM having already triggered the mucosal arm of the immune system. These experiments are ongoing. The described experiments were completed using a biologically made BECC438b, however, a synthetically made BECC438s is being trialed, which can be superior to the biologically derived version. Similarly, there is a BECC470b (and soon to be s) that can be used. Trials with the influenza vaccine have shown that BECC470 is superior to BECC438 in aged mice. Additionally, BECC438b is a biosimilar to MPLA and PHAD. It is envisioned that these TLR4 agonists can be used, if available. Experiments with PHAD can determine whether BECC438 is superior. Lastly, the ME formulation contains excipients as well as squalene. Experiments are underway to examine the need to squalene or whether another oil such as soybean oil can be used. Furthermore, a dry powder containing the L-PaF, ExlA and BECC is being produced now. This powder can be administered IN and is likely to be stable at room temperature.

### a) Materials and Methods

### (1) Patented Materials

L-PaF is as described in the previous patent. Biologically synthesized BECC438 is as described in the patent (This proposal integrates a novel technology for the production of ‘designer’ TLR4 activating ligands (i.e., MPLA mimetics) with proven adjuvanticity using model bacterial and viral antigens and translationally relevant in vivo challenge models8,20,21. The proven BECC method is an alternative route to produce lipid A mimetics quickly and efficiently and offers the advantage of ease of manipulation of the downstream immune response (Patents—U.S. Pat. No. 10,358,667, Europe 2964254).

### (2) ExlA Production

ExlA was cloned into pET9a by replacing the CTAGCATGACTGGTGGACAGCAAATGGGTCGCGGATCCGGCTGCTA sequence following the ATG of the NdeI site with the ExlA C-terminus. The exlA-pET9a plasmid was transformed into the expression strain BLR(DE3). The strain was grown in LB media to an A₆₀₀=0.8 and induced with 1 mM IPTG for 3 hours or overnight. The bacteria were collected by centrifugation, resuspended in Q column buffer (50 mM Tris-HCl, pH 7.5, 50 mM NaCl) and lysed by sonication. The suspension was clarified and the supernatant passed over a Q column equilibrated with Q buffer. The flow through was collected and loaded onto and SP column. The SP column was washed with Q column buffer and then washed with Q buffer containing 300 mM NaCl. Finally the ExlA was eluted with Q buffer containing 500 mM NaCl. It was dialyzed against PBS pH 6.5. It is stored at −80° C.

### (3) L-PaF Formulations

For the ME emulsion, squalene (8% by weight) and Polysorbate 80 (2% by weight) was mixed to achieve a homogenous oil phase. 40 mM Histidine (pH 6) and 20% sucrose were added to the oil phase and mixed with a Silverson L5M-A standard high speed mixer at 7500 RPM. This mixture was further processed (6 passes) using a Microfluidics 110P microfluidizer with pressure at 20,000 psi to generate a milky emulsion of 4XME (MedImmune Emulsion). Polysorbate 80 acted as an emulsification agent that stabilized the emulsion. BECC 438b adjuvant solution (2 mg/mL) was prepared in 0.5% triethanolamine (in water) first by vortexing, and then was sonicated for 30 minutes at 60° C. in a water bath sonicator until BECC 438b was completely dissolved. The pH of BECC 438b solution was adjusted to 7.2 with 1 M HCl. To make L-PaF-BECC-ME formulation, ME and BECC 438b was mixed first with vortexing for 2 minutes, and incubated overnight at 4° C. The next day, L-PaF was mixed with ME-BECC solution at a volumetric ratio of 1:1 to achieve desired final antigen concentration by vortexing briefly. As appropriate, ExlA was also added.

To make chitosan nanoparticle, chitosan needs to be purified. Briefly, 1 gram of chitosan was added in 10 mL of a 1 M NaOH, and stirred for 3 hours at 50° C. The chitosan solution was then filtered through 0.45 μm membrane, and the resultant pellet washed with 20 mL of MilliQ water. The recovered chitosan was resuspended in 200 mL of 1% (v/v) acetic acid solution and stirred for 1 hour. The solution was filtered through 0.45 μm membrane, and 1 M NaOH was added to adjust the pH to 8.0, resulting in purified chitosan. Purified chitosan was vacuum dried for 24 h at 40° C.

C48/80 loaded chitosan nanoparticles (Chi-C48/80 NPs) were prepared by adding dropwise 3 mL of an alkaline solution (5 mM NaOH) containing C48/80 and Na2SO4 (0.3 mg/mL and 2.03 mg/mL, respectively) to 3 mL of a chitosan solution (1 mg/mL in acetic acid 0.1%) under high-speed vortexing. The nanoparticles were formed under magnetic stirring for additional 1 hour. The formed nanoparticles were left to rest at RT for 1 hour.

The chitosan nanoparticle was centrifuged for 30 min at 4500×g, and the supernatant was discarded and the pellet was resuspended in MOPS buffer (20 mM, pH 7). The PBS buffer of L-PaF protein was also exchanged with MOPS buffer (20 mM, pH 7) using Amicon Ultra-4 centrifugal filter. BECC 438b adjuvant solution (2 mg/mL) was prepared in 0.5% triethanolamine (in water) first by vortexing, and then was sonicated for 30 minutes at 60° C. in a water bath sonicator until BECC 438b was completely dissolved. The pH of BECC 438b solution was adjusted to 7.2 with 1 M HCl. To make L-PaF-Chi-BECC formulation, chitosan nanoparticle solution was mixed with BECC 438b first with vortexing and incubated for 10 minutes. L-PaF was then added to the mixture and mixed by vortexing and incubated for 2 hours at 4° C.

### 3. Example, 3: Mouse L-SseB Formulations

A mouse experiment was started with groups vaccinated intranasally (IN): PBS, Aro (orally), L-SseB 20 μg, L-SseB 20 μg+SipD 20 μg, L-SseB 20 μg+ME, L-SseB 20 μg+ME+BECC, L-SseB 20 μg+Chi-C48/80, L-SseB 20 μg+Chi-C48/80+BECC. SipD is the tip protein of the SPI-1 type III secretion system. ME is the abbreviation of the MedImmune emulsion, which contains 20 mM Histidine, pH 6, 10% sucrose, 4% Squalene, 1% PS80. The Chi-C48/80 contains 0.15 mg/mL C48/80, 1.02 mg/mL Na2SO4, 0.5 mg/mL Chitosan. While it appears there is no interaction of the L-SseB with ME, there is an interaction of L-SseB with Chi-C48/80.

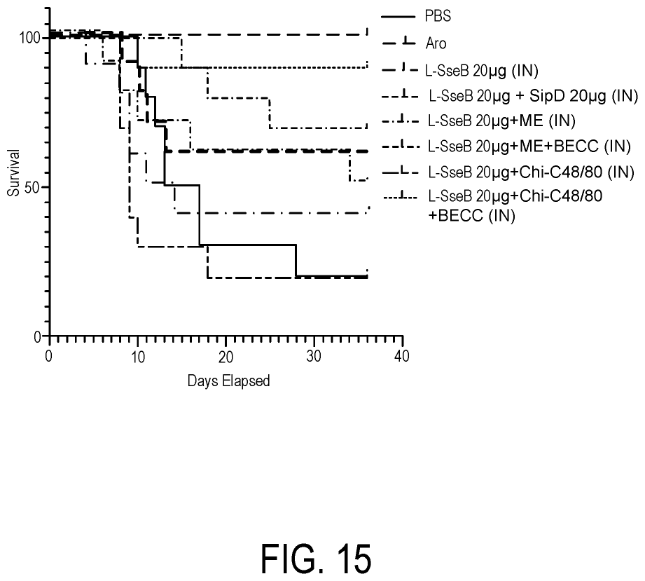

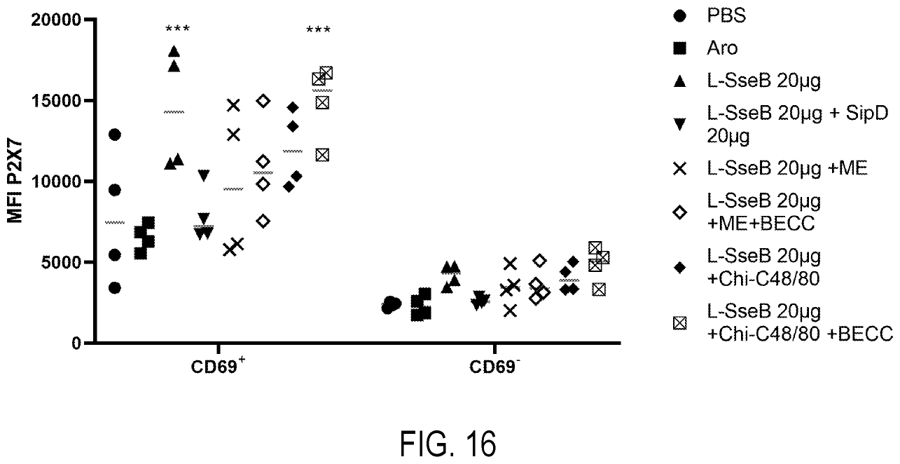

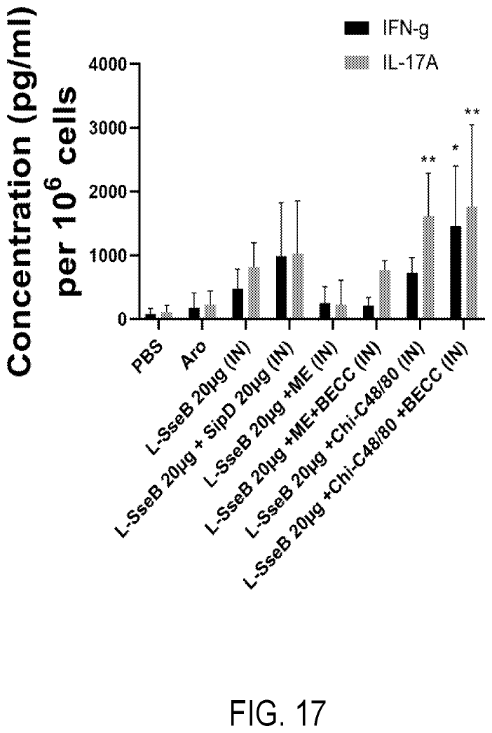

Mice were vaccinated on days 0, 14, 28 and challenged with Salmonella enterica Typhimurium on day 56 (FIG. 15). Of the PBS vaccinated mice, 80% died. Two L-SseB groups stood out: L-SseB 20 μg+ME+BECC, L-SseB 20 μg+Chi-C48/80+BECC. However, when the liver tissue resident memory cells were examined after stimulation with SseB (FIG. 16), only the L-SseB 20 μg+Chi-C48/80+BECC group (the last group) had significant levels of TRM. Similarly, when the IL-17 and IFNγ were examined (FIG. 17), the L-SseB 20 μg+Chi-C48/80+BECC group (the last group) was the only group with a formulated L-SseB that has significant levels of IL-17 and IFNγ. Thus, L-SseB-Chi-C48/80+BECC is a very promising formulation that was chosen to be tested used in rabbits.

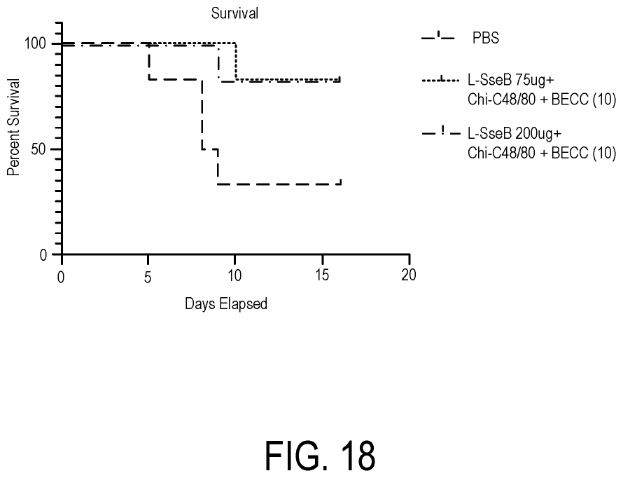

Rabbits were vaccinate 3× (day 0, 14, 28) and challenged on day 56 with Salmonella enterica Typhimurium. 4/10 PBS vaccinated rabbits died from the pathogen while only 1/6 of the L-SseB vaccinated rabbits—regardless of vaccine dose—died (FIG. 18). Furthermore, the L-SseB vaccinated rabbits regained weight faster.

### 4. Example 4: L-PaF Vaccine

L-PaF is expressed and purified. Briefly, L-PaF is overexpressed with His-tag-PcrH, the cognate chaperone for PopB. The complex is purified via standard column chromatography with detergent added to the final column to release the L-PaF from His-tag-PcrH. L-PaF is dialyzed into a stabilizing buffer containing 10 mM Histidine, pH 6, 5% sucrose, 100 mM NaCl, and 0.5% PS80. It is stored at −80. ExlA is overexpressed in E. coli and purified via sequential Q and SP columns. It is dialyzed into a stabilizing buffer of 50 mM sodium phosphate, pH 7.5, 100 mM NaCl. Both proteins have LPS levels <0.05 IU/mL (<1 EU/mg) as measured by Endosafe reader (CRL) as per manufacturer's specifications. Prior After freezing one tube of each protein is thawed to assess protein folding via CD spectroscopy, aggregation via DLS, and degradation via SDS-PAGE. The Ernst laboratory at University of Maryland-Baltimore extracts BECC438 and assessed proper structure and purity via MS.

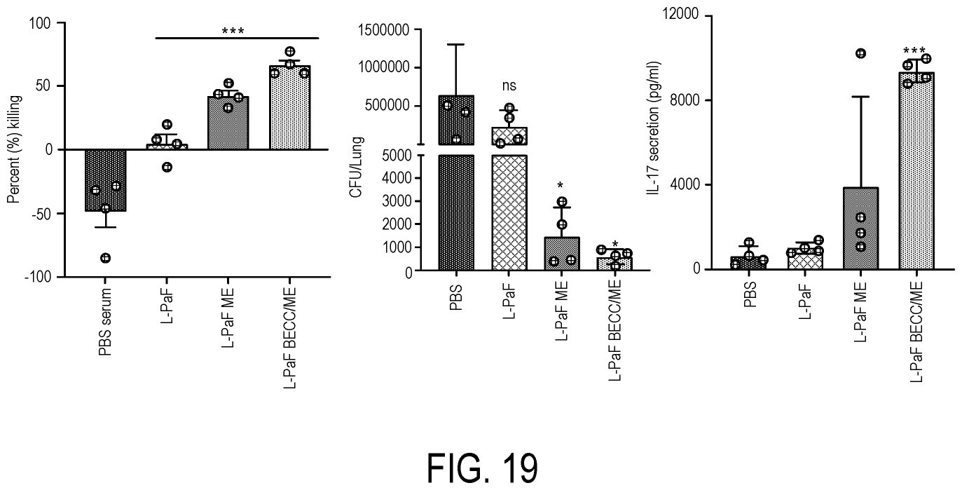

In mice and rats, L-PaF elicited important OPK and IL-17 activities, thus making it potentially useful as a prophylactic vaccine in humans—in whommonomeric subunit vaccines typically fail. Therefore, we have formulated L-PaF into two unique emulsions ±BECC438 and found that the MedImmune Emulsion (ME) formulation (10 mM Histidine, pH 6, 5% sucrose, 2% Squalene, 0.5% PS80) demonstrated the optimal efficacy (FIG. 1). ME is an oil-in-water emulsion that is ~100 nm in diameter, allowing for efficiently uptake by DCs to be transported to LNs. It should be noted that squalene-based oil-in-water emulsions have been shown to increase the efficacy of some influenza vaccines in the elderly. Mice (6-8 week old) were vaccinated IN with PBS or 1 μg L-PaF formulated with ME±BECC438 (10 μg) (FIG. 19). While sera from mice vaccinated with L-PaF alone had some OPK activity, formulating L-PaF with ME increased the killing to 47% (FIG. 19 left). Upon adding BECC, there was an additional increase in killing to 67% (FIG. 19 left). On day 56, mice were challenged with the clinical Pa strain mPa08-31 (collected from sputum of CF patient, invasive, Group A, serotype 06) (Table 6). One day post-challenge, the lungs were assessed for CFU/lung and IL-17 secretion levels. The CFU/lung decreased upon addition of ME to L-PaF and decreased further after adding BECC438 to the L-PaF/ME. (FIG. 19 middle). In parallel with the OPK activity, IL-17 levels increased with the addition of ME to L-PaF and increased further with the addition of BECC438 (FIG. 19 right). In our prior work, vaccination with 20 μg L-PaF led to significant clearance with increased OPKactivity and IL-17 levels, which decreased as the L-PaF concentrations were reduced to 10 and 1 μg. With L-PaF in the ME formulation, the antigen dose could be reduced to 1 μg. Thus, as predicted, formulation allowed for antigen dose sparing. Furthermore, the addition of BECC438 further reduced CFU burden with concomitant increases in OPK and IL-17 levels.

TABLE 6

| Strains used in | Sample | | Invasive | |
|---|---|---|---|---|
| this study | source | Group | Cytotoxic | Serotype |
| mPA08-31 | Sputum CF | A | Invasive | 06 |
| T3SS+ | patient; Birket | | | |
| PA15808959 | Sputum CF | A? | Cytotoxic | 04 |
| T3SS+ | patient; Birket | | | |
| CEC124 | acute, abdomen | C2 | N/A | 12 |
| ExIA+ | infection; Ernst | | | |
| 6077 | Eye infection; J. | A | Cytotoxic | 11 |
| T3SS+ | Goldberg(Emory) | | | |

It should be noted that the L-PaF/BECC group is not considered worthwhile as a control since it does not present antigen in an oligomeric manner, which is often the downfall of vaccines in humans. Similarly, a “PaF”+BECC groups is not presented since use of BECC without the LTA1 does not elicit the IL-17 required for protection. For these experiments, the biologically derived BECC438 was used. We have recently used a fully synthetic BECC438 (sBECC438) in our equivalent vaccine against Shigella (called L-DBF). With 5 μg of sBECC438, only 1 μg of L-DBF elicited 90% mouse survival after an otherwise lethal challenge. Thus, we expect to be able to further reduce the TRL4 adjuvant with the use of sBECC438. In this project biologic BECC438 is used due to the cost of synthesis of the BECC438s currently. The sBECC438 is currently in GMP production at Avanti, which reduces cost for us in this vaccine in the final stages.

While these results complete the proof-of-concept for a formulated L-PaF vaccine, further studies are required to include Pa strains that lack the T3SS and instead possess ExlA, which disrupts host cell membranes. We vaccinated mice on days 0, 14, 28 with PBS, 20 μg ExlA+10 μg L-PaF±ME/BECC438. As a positive control vaccine for the ExlA+ strain, Pa CEC124 (acute, abdomen infection isolate, Group C2, serotype 012), 20 μg ExlA+dmLT was used, while the positive control vaccine for the T3SS+ strain, mPa08-31, was 10 μg L-PaF. As expected, all groups vaccinated with L-PaF were protected from the mPa08-31 challenge. Importantly, all groups vaccinated with ExlA were protected from CEC124 challenge. These results are consistent with OPK results using mPa08-31 where sera from the group vaccinated with 20 μg ExlA+10 μg L-PaF killed 49% of the mPa08-31 and 25% of the CEC124 cells and sera from the group vaccinated with 20 μg ExlA+10 μg L-PaF+ME/BECC438 killed 64% of the mPa08-31 and 50% of the CEC124 cells. Thus, again in formulated L-PaFEB438 protects better than the unformulated proteins alone.

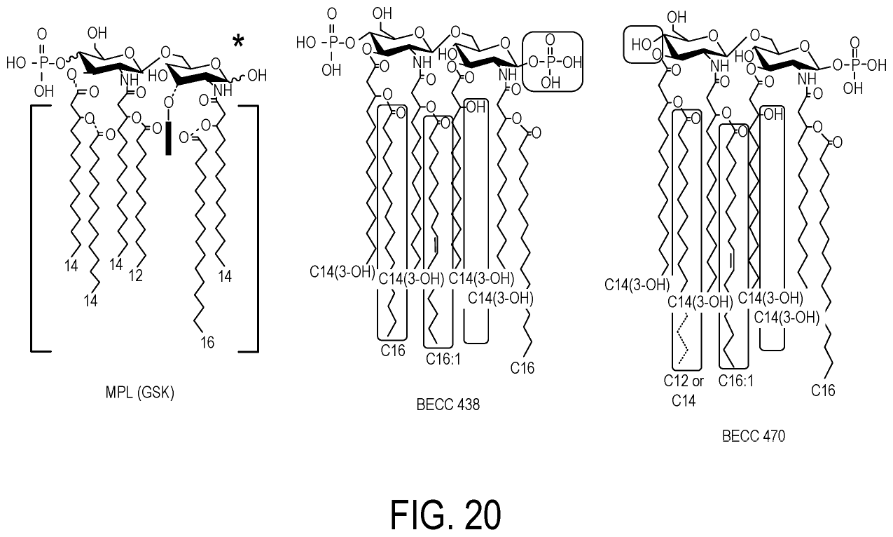

Monophosphoryl lipid A (MPL) is owned by GSK and does not provide it to the research community. Invivogen provides a version of MPL. The BECC platform was created to provide consistent defined batches of MPL biosimilars. BECC438 (bisphosphorylated) and BECC470 (monophosphorylated) (see below for usage) are hepta-acylated structures with one unsaturated fatty acid component (FIG. 20). These alterations affect molecular interactions with components of the host innate immune system (MD-2/TLR4 complex). 3D-PHAD (Avanti Polar Lipids) is a hexa-acylated synthetic monophosphorylated hexa-acylated lipid A molecule and is a commonly used control for GlaxoSmithKline's MPL. However, GSK MPL is of biological origin prepared by extraction from Salmonella Minnesota (Re595) and thus contains a mixture of multiply acylated lipid A congeners (3-7 fatty acids containing structures) arising from derivatization of MPL in the parent extract (30-32). The relative amounts of each congener group in MPL is extensive: tetraacyl: 15 to 35%; -pentaacyl: 35 to 60%; -hexaacy: 20 to 40%; -heptaacyl: less than 0.5%. In contrast, the BECC438b molecules are defined and the hexa-acylated BECC438s version has been chemically synthesized and has shown no acutetoxicity in mice, rats or rabbits.

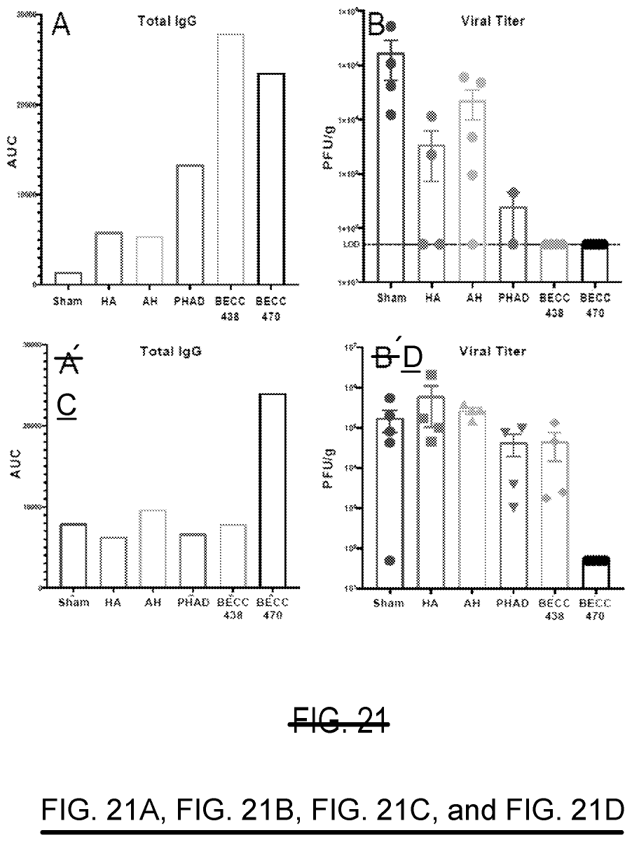

In addition to our Pa results, Dr. Bob Ernst (collaborator on this project, see letter of support) has compared these TLR4 agonists in a Y. pestis challenge model and has shown BECC438 is superior to PHAD and MPL. However, with respect to this project, Dr. Ernst has recently compared the responses of young and middle aged mice (12 month) using a mouse influenza model (FIG. 21). In addition to BECC438 (bisphosphorylated), We have also employed BECC470 (monophosphorylated) as an adjuvant with the well-characterized influenza hemagglutinin protein (recombinant protein derived from A/California/04/2009 (H1N1 Cal/09) in a murine prime/boost vaccination schedule. Using a homologous influenza challenge model, he demonstrated higher IgG titers in mice vaccinated using the BECC adjuvants compared to using Alhydrogel (AH) and PHAD as the adjuvant (FIG. 21 top). Similarly, the BECC438/HA and BECC470/HA formulations showed superior protection over PHAD or AH (FIG. 21 top). Dose-dependent protection from influenza challenge was also observed and superior protection was attained with as low as 40 ng of HA (equivalent to 7.5 μg HA per serotype in human quadrivalent vaccines).

In aged populations, immunosenescence leads to a progressive decline in the innate and adaptive immune responses. The ability of a vaccine formulation to overcome this is a significant advancement in vaccinology for use in older adults. Using 12-month-old mice (representing middle aged adults) immunized using a prime/boost schedule, BECC438 and BECC470 were used, as well as the PHAD and AH to vaccinate mice (FIG. 21 bottom). In contrast to younger mice, only BECC470/HA elicited increased total IgG and undetectable viral titers as compared to PHAD, AH and BECC438 (similarly formulated) immunized mice (FIG. 21 bottom). The difference in efficacy between the two BECC molecules can be due to the engineered loss of the 1 position phosphate on BECC470 that results in altered binding to the MD-2/TLR4 receptor complex, versus the bis-phosphorylated BECC438 (FIG. 20).

### E. REFERENCES

Almagro, P., M. Salvado, C. Garcia-Vidal, M. Rodriguez-Carballeira, E. Cuchi, J. Torres and J. L. Heredia (2012). “Pseudomonas aeruginosa and mortality after hospital admission for chronic obstructive pulmonary disease.” Respiration 84(1): 36-43.
Anantharajah, A., M. P. Mingeot-Leclercq and F. Van Bambeke (2016). “Targeting the Type Three Secretion System in Pseudomonas aeruginosa.” Trends Pharmacol Sci 37(9): 734-749.
Baker, S. M., J. B. McLachlan and L. A. Morici (2020). “Immunological considerations in the development of Pseudomonas aeruginosa vaccines.” Hum Vaccin Immunother 16(2): 412-418.
Bento, D., H. F. Staats, T. Goncalves and O. Borges (2015). “Development of a novel adjuvanted nasal vaccine: C48/80 associated with chitosan nanoparticles as a path to enhance mucosal immunity.” Eur J Pharm Biopharm 93: 149-164.
CDC (2019). Antibiotic Resistance Threats in the United States. Atlanta, GA, U.S. Department of Health and Human Services, CDC; 2019.
Corthesy, B. and G. Bioley (2018). “Lipid-Based Particles: Versatile Delivery Systems for Mucosal Vaccination against Infection.” Front Immunol 9: 431.
Das, S., D. R. Howlader, Q. Zheng, S. S. K. Ratnakaram, S. K. Whittier, T. Lu, J. D. Keith, W. D. Picking, S. E. Birket and W. L. Picking (2020). “Development of a Broadly Protective, Self-Adjuvanting Subunit Vaccine to Prevent Infections by Pseudomonas aeruginosa.” Front Immunol 11: 583008.
Elsen S, Huber P, Bouillot S, Coute Y, Fournier P, Dubois Y, Timsit J F, Maurin M, Attree I. A type III secretion negative clinical strain of Pseudomonas aeruginosa employs a two-partner secreted exolysin to induce hemorrhagic pneumonia. Cell Host Microbe. 2014; 15(2):164-76. Epub 2014/02/18. doi: 10.1016/j.chom.2014.01.003. PubMed PMID: 24528863.
Gan, Q. and T. Wang (2007). “Chitosan nanoparticle as protein delivery carrier-systematic examination of fabrication conditions for efficient loading and release.” Colloids Surf B Biointerfaces 59(1): 24-34.
Goure, J., A. Pastor, E. Faudry, J. Chabert, A. Dessen and I. Attree (2004). “The V antigen of Pseudomonas aeruginosa is required for assembly of the functional PopB/PopD translocation pore in host cell membranes.” Infect Immun 72(8): 4741-4750.
Gregg, K. A., E. Harberts, F. M. Gardner, M. R. Pelletier, C. Cayatte, L. Yu, M. P. McCarthy, J.

D. Marshall and R. K. Ernst (2018). “A lipid A-based TLR4 mimetic effectively adjuvants a Yersinia pestis rF-V1 subunit vaccine in a murine challenge model.” Vaccine 36(28): 4023-4031.
Grimwood, K., J. M. Kyd, S. J. Owen, H. M. Massa and A. W. Cripps (2015). “Vaccination against respiratory Pseudomonas aeruginosa infection.” Hum Vaccin Immunother 11(1): 14-20.
Lefeber, D. J., B. Benaissa-Trouw, J. F. Vliegenthart, J. P. Kamerling, W. T. Jansen, K. Kraaijeveld and H. Snippe (2003). “Th1-directing adjuvants increase the immunogenicity of oligosaccharide-protein conjugate vaccines related to Streptococcus pneumoniae type 3.” Infect Immun 71(12): 6915-6920.
Loebinger, M. R., A. U. Wells, D. M. Hansell, N. Chinyanganya, A. Devaraj, M. Meister and R. Wilson (2009). “Mortality in bronchiectasis: a long-term study assessing the factors influencing survival.” Eur Respir J 34(4): 843-849.
Marasini, N., M. Skwarczynski and I. Toth (2017). “Intranasal delivery of nanoparticle-based vaccines.” Ther Deliv 8(3): 151-167.
Martinez-Becerra, F. J., J. M. Kissmann, J. Diaz-McNair, S. P. Choudhari, A. M. Quick, G. Mellado-Sanchez, J. D. Clements, M. F. Pasetti and W. L. Picking (2012). “Broadly protective Shigella vaccine based on type III secretion apparatus proteins.” Infect Immun 80(3): 1222-1231.
Merakou, C., M. M. Schaefers and G. P. Priebe (2018). “Progress Toward the Elusive Pseudomonas aeruginosa Vaccine.” Surg Infect (Larchmt) 19(8): 757-768.
Norton, E. B., L. B. Lawson, Z. Mahdi, L. C. Freytag and J. D. Clements (2012). “The A subunit of Escherichia coli heat-labile enterotoxin functions as a mucosal adjuvant and promotes IgG2a, IgA, and Th17 responses to vaccine antigens.” Infect Immun 80(7): 2426-2435.
Reboud E, Basso P, Maillard A P, Huber P, Attree I. Exolysin Shapes the Virulence of Pseudomonas aeruginosa Clonal Outliers. Toxins (Basel). 2017; 9(11). Epub 2017/11/10. doi: 10.3390/toxins9110364. PubMed PMID: 29120408; PMCID: PMC5705979.
Sainz-Mejias, M., I. Jurado-Martin and S. McClean (2020). “Understanding Pseudomonas aeruginosa-Host Interactions: The Ongoing Quest for an Efficacious Vaccine.” Cells 9(12).
Valli E, Harriett A J, Nowakowska M K, Baudier R L, Provosty W B, McSween Z, Lawson L B, Nakanishi Y, Norton E B. LTA1 is a safe, intranasal enterotoxin-based adjuvant that improves vaccine protection against influenza in young, old and B-cell-depleted (muMT) mice. Sci Rep. 2019; 9(1):15128. Epub 2019/10/24. doi: 10.1038/s41598-019-51356-w. PubMed PMID: 31641151; PMCID: PMC6805908.
Wagner, A. a. W., B. (2020). “Vaccines to Prevent Infectious Diseases in the Older Population: Immunological Challenges and Future Perspectives.” Frontiers in Immunology 11.
Wang, Y., X. Cheng, C. Wan, J. Wei, C. Gao, Y. Zhang, H. Zeng, L. Peng, P. Luo, D. Lu, Q. Zou and J. Gu (2021). “Development of a Chimeric Vaccine Against Pseudomonas aeruginosa Based on the Th17-Stimulating Epitopes of PcrV and AmpC.” Frontiers in Immunology 11(3538).
Wei, Y., J. Xiong, N. R. Larson, V. Iyer, G. Sanyal, S. B. Joshi, D. B. Volkin and C. R. Middaugh (2018). “Effect of 2 Emulsion-Based Adjuvants on the Structure and Thermal Stability of Staphylococcus aureus Alpha-Toxin.” J Pharm Sci 107(9): 2325-2334.
Wei, Y., N. R. Larson, S. K. Angalakurthi and C. Russell Middaugh (2018). “Improved Fluorescence Methods for High-Throughput Protein Formulation Screening.” SLAS Technol 23(6): 516-528.
Winsor, G. L., E. J. Griffiths, R. Lo, B. K. Dhillon, J. A. Shay and F. S. Brinkman (2016). “Enhanced annotations and features for comparing thousands of Pseudomonas genomes in the Pseudomonas genome database.” Nucleic Acids Res 44(D1): D646-653.

### F. SEQUENCES

ExlA Full length nt
SEQ ID NO: 1
ATGCATAGAGACAATCCTGTCTTCCACCTCTCCCCCCGGGGAAAACTCCGTTGCCTG
ATCGCCGGCCTGCTGCTGGCGCCCCACCTGCCCCAGGCTTTCGCCGGCGGGCTGGAG
GCGGCCGGCGGGCCGGGCGGCACGCCGCAACTGCTCAACCAGGGCGGCGTGCCCAT
CGTCAATATCGTCGCGCCGAACGCCGCCGGCCTGTCGCACAACCAGTTCCTCGACTA
CAACGTCGACCGCCAGGGCCTGGTCCTCAACAATGCGCTGCAAGCCGGCGCCTCGC
AGCTCGCCGGACAACTGGCGGCCAACCCGCAGCTGCGCGGCGACGCCGCCAGTGCC
ATCCTCAACGAAGTGATCAGCCGCAACGCCTCGGCGATCAACGGCCCGCAGGAAAT
CTTCGGCCAGGCCGCCGACTACATCCTGGCCAACCCCAACGGCATCTCGGTGAACG
GCGGCAGCTTCATCAACACGCCCCATGCCAGCCTGCTGGTCGGCCGTCCGGAACTG
GCCGACGGCAAGCTGCAGGCGCTGAACACCAACGACGCCGTCGGCGCCCTGCAGAT
CCAGGGCCAGGGCCTGAGCAACCGGGATGGCAGCATCGCCCTGCTGGCGCCGCGAG
TCGACAGCCAAGGCAAGATCGAGGCGTCGGCCGAGCTCGACCTGACCGTCGGTCGC
AACCGCATCGACTACCCGAGCGGCAAGGTCGAGCGCGACCCGTCCGGCGACGTCCG
TCCGGGCGAGCGGCGCATCGACGCCAGCCTGTTCGGCGCCATGCAGGCCGGACGCA
TCAACATCCTCAGCACCGCCGAGGGCGCCGGCGTGCGCGTCGGCCCGGTCGGGATC
GACGGCAGGGACGGCGTCGACCTGCGCTCCGCCGGCGACCTGTCGATCAGCGGCCA
GGCGCTTCCCGACAACAGCCTGAACGCCTTGCGCGCGGCGATCCGCAGCGACAGCG
GCAACGTCGGCCTGCATGCCCGCGGCGACCTGAGCCTGGCCGCCGCCGACGTCAGC
GGCGGCAGGGTCGACCTCAAGTCCGGGCGCAACCTGACCCTGGGCAGCGTGGAAAG
CCGCAACCTGCGGGAAAGCCGCGAGCGCTGGAGCAACAGCACCATCGGCATCACCT
GGGAAACCTACGACCGCACGCGTACCGTCACCGATTCGAAGCAGCACGGCAGCCGG
ATCGATGCCCGCGCCGACGCGAGCCTGGCCGCGCGGGGCGACAGCGAACTGAGGG
CGGCGACGGTCAAGGCCGGCGCTACCCTGAAAGTCTCCAGCGGCGGCGATACCCGT
CTTCTCGCCGCCACCGAGACCCGCACCGAACGCGACCAGGGCGCGCACCGCAAGCA
CCTGTGGAAAGCCAACTGGGACAAGGGCAGCAGCGAGCAACGCAGCGTGGCCAGC
AGCCTCGAAGGCGCGCGGGTCGAACTCGGCGGCGGCCGGCGCCTGAATCTGGAAGG
CGCCGACGTGGCCAGCCGCGGCGACCTCGACCTGCAAGCCAAGAGCGTCGACATCG
GCAGCGCCAGCCGCAGCCACAGCAGCCGCGACAACAGCTACTCCGGCGACCTGGTC
GGCGGCAGCTTCTTCGGCAGCCACGGCGATGGCGACAGCGGCAAGACCCTGCAACA
GGGCAGCCGGGTCAAGGCCGACGGCGCACTGACGGTGACCGCCGATGCGGTCGAG
GTGCGCGGCAGCCAGGTACGCGGCGCGCGCAAGGCCGAGGTAGTCAGCGGGAAAG
GCTCGCTGCGCATCGACGGCGTGGAGGAAACCGCCCACAGCAACAGCTACAGCAAG
GACAGCAAGTTCTTCGGCATCGCCAAGGACGAGAGTCGCCAGCGCAGCAAGGACA
GCAGCAACCGCGCCAGCGAAGTACGCTCGGACAGCAACCTGACCCTGCGCAGCGCC
GCCGACATCGCCATTCGCGGCTCCCGGGTCGAGGCTGGCGGCGCCCTCGCCGCCGA
GGCCAAGGGCAACCTGGAGATAGCCTCGGCGCAGGAGCGCCACGACGGCAACGAC
AGCCGCCACACCCGCGGCTTCGACGCCTATGCCGGCGAGCAGACCCCAGGCAGCCG
CCAATACCGCGCCGGGGTGCGCTACCAGGACCAGCGGACCAGCGTCCGCCGCGAGG
AAACCCGCAACAGCGGCTCCAGCCTGGGCGGCGCCTCCCTGGCCGTGAAGGCCGGC
GGCGATCTCACCGTGAAGGGGGCCGAGCTGAAGGCCAGCGCGGGCGACGCCAGCC
TCTCCGGGAAGAACGTCGCCCTGCTCGCCGAGCAGGACAGCAAGACCCGCAGCAGC
GAACAGACCACCACCGGCGGCGGCTTCTACTATACCGGCGGCCTGGACCGTGCCGG
CAGCGGTATCGAAGTCGGCCACCAGCGGATCGACGAGAACGACGCCGAAAGCCAT
GCGCGCACCAGCCAGGTGAACGCGACGGGCAATCTCAGGATCGACGCCGCCCAGG
GCAGCCTGACGACGCAGGGCGCGCGCCTGGAGGCCGGCGACAGCCTGGCGGTCGCC
GCCGGCACGGTCGACAACCAGGCCGCCCGCGACAGCCAGAGCAGCCAGCGCCACG
ACAGCGGCTGGAGCGGCGACATCGGCGCCAACCTCGAGTACCGCGGCATCGCCCGG
CCGATCGAGAAGGCGGTCGAAGGCGTCGCCCAGCGCAAGGTCCACCAGCCCGGCCT
GCTCGACAACCTGGAGCAGCCGAACGTCGGCGTCGACCTCGAGATCAGCCACCGCG
ACAGCCGCGGCGAGCAACAGGCGAGCCAGGCGCAGGTCAGCAGCTTCGCGGGCGG
CCAGGTCGAACTGAAGGTCGGCGACGCCCTGCGGGACGAGGGCACCCGTTACCAGG
CTCGCAGCGGAGGCCTCCTCATCGACGCCGCCAGGCATGACGCCAGGGCGGCGGAG
AACACCTCCGGCAGCCATGAGCAGAGCCTCGACGCCAAGGTCGGCGGGCGCCTCTA
CACCACCACCGGCCAGGACCTGAACCTGCGCCTGAGCGGCATTGGCGGCAGCAGCG
AGAACAGCGCCAGCCAGACCACCGCGGTGGTCGGCGAATACGCCGCGAAGCAGGG
CGTCGAGATCCGCCTCGGCGGAGACGGCCTCTACCAGGGCAGCCGCTTCGACGGCG
GCGAAGCCGGGGTCAGGCTCAGCGCCGGCGGCAACCTGGCCCTGGAACAGGCCAA
CGACCGGCAGAGCGCCAGCAGCGCCAGCCTGCGTGGCGACGCCGCGTTGAGCGGCG
GCATGGCCCCCAGCGCCAACGGCAAAGGGCTGAACGCCAGCGCCGGCCTGCAACTC
GACCACAAGGCCGGCGACAGCCGGGACAGCCAGGCGCGGGTCGCCGACATCCAGG
CCAAGGGCGCGGTGGAGCTGCGCAGCGGCGGCGATCTGGTCCTGCAAGGCAGCAAT
ATCGGCAGCGCGGCAGCGAAGACCGGCGACATAGTCCTGGCCGCCGGCGGCAAGCT
CGACCTGCAGGCCGCCCGCGATAGCCACCGGGCCGGGGGAAACAACCTCGGCGGC
GGCTTCAGCCTAGGCGGCGGCAGCGTTCGCGACGCCGAAACGAGCAGCAAGAACG
GCAGCGTCAGCGGCAACTTCAACATCGGCCGGGTCGACGAGGAACGTCACGCGCTG
AACGGCGGCAACCTGCACAGCGCGACCAAGGCCAGCCTTTCCAGCGCCGCAGACGA
CGCCACCGCGGTACGCCTGCAAGGCACTCGCATCGAAGCCGCCCAGGTCAGCCTCG
AAGCCGGCAACGGCGGCATTCTCCAGGAGTCCGCCGAATCCAGCGAGCGGCGCGAC
AACTGGGGCGTGCTGCTCGGTGCCGGGGCCAACGGCGGCAAGACCACCGGCGCGCC
GAGCGACTACCGGAGCGACTATGCCGTCCAGGCCCGCGCCAAGGTCGATGTCGATG
TCCTGCGCAGCCAGACCCAGGGCGACAGCGTCATCCAGGCCGACCGGGTAATCCTG
GCGAGCCAGGGCGACACCCGCCTGGAGGGCGCGCGCATCGACGCGGCACAGGTGG
ACGGGCGCATCGGCGGCGACCTGCGGGTGGAGAGCCGCCAGGACCGCGCGGAGGG
CGTGAAGGTCAACGTCGACGCGCGCCTGGGCGTGGAGAAGAACCAGCCCGGCCTGG
TGAACAAGCTGGCGAGCAAGACCGGACCGTTGAAGGACAAGCTGGAAACCAAGGC
CGAGAATGCTTTCGACAAGCACCGCGGCAAGTTGGAGAACGGCATCGACCGTAATG
TCGAGCGGCTCGGCAAGGCCGGGGACAACCTCCTCGCCAAAGCCGAAAAGGCCAA
GGAGCGCCTGGGCGAGAAGCTGGTCCGCAGCGGCAGCTACGAGGTCAACCCGGAG
CCGCGCGGCGCCTTCGCCAGCAAGCTGGACAGGGCCAGGGGCTATCTGGCGGAGAA
AGGCGAAGCGCTCGGCGACCGGCTGTCCGGCCTCAAGCAGCGCCTGTCGCCGAACA
AGACCGGTAGCTATGTGGTGAACGACAAGCAGACGGCCGGCGCCAAGGTCGGCAA
TGCCGCCGAGAACGTGCTGTTCGGCGACAAGAGCGGCGAAGCCTCGGTAACCCCGA
CGCTGTACCTGGACGTCAGCCACGTCAGCCGCAACTACGTCACCGAGGCCTCCGGC
ATCACCGGCAGGCAGGGCGTGAACCTGCAGGTGGGCGCAGCGACCCAGCTGACCG
GCGCACGGATCAGCGCCAGCGACGGCAAGGTCGACCTCGGCGGCTCGCGCGTGGAA
ACCCGCGCCCTGGCCGGCAAGGACTACCGCGCCGATCTCGGCCTGAACGTCTCCAG
GTCGCCGGTGGACCTGGCCTTCGGTATCAAGGACGAGTTCAGCCAGGAGCACGACC
AGGCGACCCGCGACGACCAGGCCTTCAACCTCGGCGCCCTGCGCGTCGGCGGACGC
AACCGCGACCAGCAGTTGCAGGCCGGCATCGAGCAGAAGGCCGACTGA
ExlA Full length AA
SEQ ID NO: 2
MHRDNPVFHLSPRGKLRCLIAGLLLAPHLPQAFAGGLEAAGGPGGTPQLLNQGGVPIVN
IVAPNAAGLSHNQFLDYNVDRQGLVLNNALQAGASQLAGQLAANPQLRGDAASAILN
EVISRNASAINGPQEIFGQAADYILANPNGISVNGGSFINTPHASLLVGRPELADGKLQAL
NTNDAVGALQIQGQGLSNRDGSIALLAPRVDSQGKIEASAELDLTVGRNRIDYPSGKVE
RDPSGDVRPGERRIDASLFGAMQAGRINILSTAEGAGVRVGPVGIDGRDGVDLRSAGDL
SISGQALPDNSLNALRAAIRSDSGNVGLHARGDLSLAAADVSGGRVDLKSGRNLTLGSV
ESRNLRESRERWSNSTIGITWETYDRTRTVTDSKQHGSRIDARADASLAARGDSELRAA
TVKAGATLKVSSGGDTRLLAATETRTERDQGAHRKHLWKANWDKGSSEQRSVASSLE
GARVELGGGRRLNLEGADVASRGDLDLQAKSVDIGSASRSHSSRDNSYSGDLVGGSFF
GSHGDGDSGKTLQQGSRVKADGALTVTADAVEVRGSQVRGARKAEVVSGKGSLRIDG
VEETAHSNSYSKDSKFFGIAKDESRQRSKDSSNRASEVRSDSNLTLRSAADIAIRGSRVE
AGGALAAEAKGNLEIASAQERHDGNDSRHTRGFDAYAGEQTPGSRQYRAGVRYQDQR
TSVRREETRNSGSSLGGASLAVKAGGDLTVKGAELKASAGDASLSGKNVALLAEQDSK
TRSSEQTTTGGGFYYTGGLDRAGSGIEVGHQRIDENDAESHARTSQVNATGNLRIDAAQ
GSLTTQGARLEAGDSLAVAAGTVDNQAARDSQSSQRHDSGWSGDIGANLEYRGIARPI
EKAVEGVAQRKVHQPGLLDNLEQPNVGVDLEISHRDSRGEQQASQAQVSSFAGGQVEL
KVGDALRDEGTRYQARSGGLLIDAARHDARAAENTSGSHEQSLDAKVGGRLYTTTGQ
DLNLRLSGIGGSSENSASQTTAVVGEYAAKQGVEIRLGGDGLYQGSRFDGGEAGVRLS
AGGNLALEQANDRQSASSASLRGDAALSGGMAPSANGKGLNASAGLQLDHKAGDSRD
SQARVADIQAKGAVELRSGGDLVLQGSNIGSAAAKTGDIVLAAGGKLDLQAARDSHRA
GGNNLGGGFSLGGGSVRDAETSSKNGSVSGNFNIGRVDEERHALNGGNLHSATKASLS
SAADDATAVRLQGTRIEAAQVSLEAGNGGILQESAESSERRDNWGVLLGAGANGGKTT
GAPSDYRSDYAVQARAKVDVDVLRSQTQGDSVIQADRVILASQGDTRLEGARIDAAQV
DGRIGGDLRVESRQDRAEGVKVNVDARLGVEKNQPGLVNKLASKTGPLKDKLETKAE
NAFDKHRGKLENGIDRNVERLGKAGDNLLAKAEKAKERLGEKLVRSGSYEVNPEPRG
AFASKLDRARGYLAEKGEALGDRLSGLKQRLSPNKTGSYVVNDKQTAGAKVGNAAEN
VLFGDKSGEASVTPTLYLDVSHVSRNYVTEASGITGRQGVNLQVGAATQLTGARISASD
GKVDLGGSRVETRALAGKDYRADLGLNVSRSPVDLAFGIKDEFSQEHDQATRDDQAFN
LGALRVGGRNRDQQLQAGIEQKAD
ExlA C-term nt
SEQ ID NO: 3
GTCAACGTCGACGCGCGCCTGGGCGTGGAGAAGAACCAGCCCGGCCTGGTGAACAA
GCTGGCGAGCAAGACCGGACCGTTGAAGGACAAGCTGGAAACCAAGGCCGAGAAT
GCTTTCGACAAGCACCGCGGCAAGTTGGAGAACGGCATCGACCGTAATGTCGAGCG
GCTCGGCAAGGCCGGGGACAACCTCCTCGCCAAAGCCGAAAAGGCCAAGGAGCGC
CTGGGCGAGAAGCTGGTCCGCAGCGGCAGCTACGAGGTCAACCCGGAGCCGCGCG
GCGCCTTCGCCAGCAAGCTGGACAGGGCCAGGGGCTATCTGGCGGAGAAAGGCGA
AGCGCTCGGCGACCGGCTGTCCGGCCTCAAGCAGCGCCTGTCGCCGAACAAGACCG
GTAGCTATGTGGTGAACGACAAGCAGACGGCCGGCGCCAAGGTCGGCAATGCCGCC
GAGAACGTGCTGTTCGGCGACAAGAGCGGCGAAGCCTCGGTAACCCCGACGCTGTA
CCTGGACGTCAGCCACGTCAGCCGCAACTACGTCACCGAGGCCTCCGGCATCACCG
GCAGGCAGGGCGTGAACCTGCAGGTGGGCGCAGCGACCCAGCTGACCGGCGCACG
GATCAGCGCCAGCGACGGCAAGGTCGACCTCGGCGGCTCGCGCGTGGAAACCCGCG
CCCTGGCCGGCAAGGACTACCGCGCCGATCTCGGCCTGAACGTCTCCAGGTCGCCG
GTGGACCTGGCCTTCGGTATCAAGGACGAGTTCAGCCAGGAGCACGACCAGGCGAC
CCGCGACGACCAGGCCTTCAACCTCGGCGCCCTGCGCGTCGGCGGACGCAACCGCG
ACCAGCAGTTGCAGGCCGGCATCGAGCAGAAGGCCGACTGA
ExlA C-term AA
SEQ ID NO: 4
VNVDARLGVEKNQPGLVNKLASKTGPLKDKLETKAENAFDKHRGKLENGIDRNVERL
GKAGDNLLAKAEKAKERLGEKLVRSGSYEVNPEPRGAFASKLDRARGYLAEKGEALG
DRLSGLKQRLSPNKTGSYVVNDKQTAGAKVGNAAENVLFGDKSGEASVTPTLYLDVS
HVSRNYVTEASGITGRQGVNLQVGAATQLTGARISASDGKVDLGGSRVETRALAGKDY
RADLGLNVSRSPVDLAFGIKDEFSQEHDQATRDDQAFNLGALRVGGRNRDQQLQAGIE
QKAD
PaF nucleic acid sequence
SEQ ID NO: 5
CATATGGAAGTCAGAAACCTTAATGCCGCTCGCGAGCTGTTCCTGGACGAGCTCCTG
GCCGCGTCGGCGGCGCCTGCCAGTGCCGAGCAGGAGGAACTGCTGGCCCTGTTGCG
CAGCGAGCGGATCGTGCTGGCCCACGCCGGCCAGCCGCTGAGCGAGGCGCAAGTGC
TCAAGGCGCTCGCCTGGTTGCTCGCGGCCAATCCGTCCGCGCCTCCGGGGCAGGGC
CTCGAGGTACTCCGCGAAGTCCTGCAGGCACGTCGGCAGCCCGGTGCGCAGTGGGA
TCTGCGTGAGTTCCTGGTGTCGGCCTATTTCAGCCTGCACGGGCGTCTCGACGAGGA
TGTCATCGGTGTCTACAAGGATGTCCTGCAGACCCAGGACGGCAAGCGCAAGGCGC
TGCTCGACGAGCTCAAGGCGCTGACCGCGGAGTTGAAGGTCTACAGCGTGATCCAG
TCGCAGATCAACGCCGCGCTGTCGGCCAGGCAGGGCATCAGGATCGACGCTGGCGG
TATCGATCTGGTCGACCCCACGCTATATGGCTATGCCGTCGGCGATCCCAGGTGGAA
GGACAGCCCCGAGTATGCGCTGCTGAGCAATCTGGATACCTTCAGCGGCAAGCTGT
CGATCAAGGATTTTCTCAGCGGCTCGCCGAAGCAGAGCGGGGAACTCAAGGGCCTC
AGCGATGAGTACCCCTTCGAGAAGGACAACAACCCGGTCGGCAATTTCGCCACCAC
GGTGAGCGACCGCTCGCGTCCGCTGAACGACAAGGTCAACGAGAAGACCACCCTGC
TCAACGACACCAGCTCCCGCTACAACTCGGCGGTCGAGGCGCTCAACCGCTTCATCC
AGAAATACGACAGCGTCCTGAGCGACATTCTCAGCGCGATCGGATCCATGAACCCG
ATTACGCTGGAACGTGCTGGTCTGCCGTATGGTGTTGCCGATGCTGGTGACATCCCG
GCTCTGGGTCGCCCGGTCGCACGTGATGTGGAAAGTCTGCGTGTTGAACGTCTGGCA
GCACCGGCAGCTGCAAGCGCATCTGGCACCGGTGTCGCTCTGACGCCGCCGTCTGC
AGCAAGTCAGCAACGTCTGGAAGTTGCTAACCGCGCGGAAATTGCCTCACTGGTCC
AGGCAGTGGGTGAAGACGTGGGTCTGGCACGTCAAGTGGTTCTGGCAGGTGCATCG
ACCCTGCTGAGCGCAGGTCTGATGTCGCCGCAGGCGTTCGAAATTGAACTGGCCAA
AATCACCGGCGAAGTTGAAAATCAGCAGAAAAAACTGAAACTGACGGAAATCGAA
CAGGCCCGTAAACAGAACCTGCAAAAAATGGAAGATAACCAGCAAAAAATCCGCG
AATCGGAAGAAGCTGCGAAAGAAGCGCAGAAAAGCGGCCTGGCCGCAAAAATTTT
TGGTTGGATTTCTGCTATCGCGAGTATTATCGTGGGTGCAATCATGGTTGCAACCGG
TGTCGGTGCTGCAGCAGGTGCACTGATGATTGCTGGCGGTGTCATGGGTGTCGTGAG
TCAGTCCGTGCAGCAAGCAGCTGCGGATGGTCTGATCTCAAAAGAAGTGATGGAAA
AACTGGGCCCGGCCCTGATGGGTATTGAAATGGCCGTGGCACTGCTGGCCGCAGTT
GTCTCCTTTGGTGGTTCAGCAGTTGGTGGTCTGGCACGTCTGGGTGCAAAAATCGGC
GGTAAAGCTGCGGAAATGACGGCATCCCTGGCTTCAAAAGTGGCAGACCTGGGCGG
TAAATTCGGCTCTCTGGCGGGCCAGTCACTGTCGCATAGCCTGAAACTGGGTGTGCA
AGTTTCTGATCTGACCCTGGACGTTGCAAACGGCGCCGCACAGGCTACGCACAGTG
GTTTTCAAGCGAAAGCTGCGAATCGTCAGGCCGATGTTCAAGAATCCCGTGCAGAC
CTGACCACGCTGCAGGGTGTCATTGAACGTCTGAAAGAAGAACTGAGCCGCATGCT
GGAAGCCTTTCAGGAAATTATGGAACGCATCTTCGCAATGCTGCAAGCGAAAGGCG
AAACCCTGCACAATCTGTCTTCCCGTCCGGCGGCTATCTGAGGATCC
PaF amino acid sequence
SEQ ID NO: 6
MEVRNLNAARELFLDELLAASAAPASAEQEELLALLRSERIVLAHAGQPLSEAQVLKAL
AWLLAANPSAPPGQGLEVLREVLQARRQPGAQWDLREFLVSAYFSLHGRLDEDVIGVY
KDVLQTQDGKRKALLDELKALTAELKVYSVIQSQINAALSARQGIRIDAGGIDLVDPTL
YGYAVGDPRWKDSPEYALLSNLDTFSGKLSIKDFLSGSPKQSGELKGLSDEYPFEKDNN
PVGNFATTVSDRSRPLNDKVNEKTTLLNDTSSRYNSAVEALNRFIQKYDSVLSDILSAIG
SMNPITLERAGLPYGVADAGDIPALGRPVARDVESLRVERLAAPAAASASGTGVALTPP
SAASQQRLEVANRAEIASLVQAVGEDVGLARQVVLAGASTLLSAGLMSPQAFEIELAKI
TGEVENQQKKLKLTEIEQARKQNLQKMEDNQQKIRESEEAAKEAQKSGLAAKIFGWIS
AIASIIVGAIMVATGVGAAAGALMIAGGVMGVVSQSVQQAAADGLISKEVMEKLGPAL
MGIEMAVALLAAVVSFGGSAVGGLARLGAKIGGKAAEMTASLASKVADLGGKFGSLA
GQSLSHSLKLGVQVSDLTLDVANGAAQATHSGFQAKAANRQADVQESRADLTTLQGV
IERLKEELSRMLEAFQEIMERIFAMLQAKGETLHNLSSRPAAI
LTA1-PaF nucleic acid sequence
SEQ ID NO: 7
CATatggacaatggcgatcgtttataccgtgccgactcgcgtcccccagatgagattaaacgtagcggtgggttaatgccacgtggg
caatgagtattttgaccgtggaacacagatgaacattaacctttacgatcatgcccgtgggacccagaccgggtttgtccgttatgatga
cgggtatgttagtacgagtttgtccttacgctccgcacaccttgcgggacaaagtattttatcaggctacagcacatattacatttatgt
gatcgccactgccccaaacatgttcaatgtgaacgatgtgttgggggtttacagcccccatccatatgaacaagaagtctcggcccttgg
ggggatcccatatagccagatttatggttggtaccgcgtaaattttggtgtgattgatgaacgtttgcatcgtaaccgtgaataccgcga
tcgctactaccgtaacttgaacattgcacctgccgaggacggctatcgtttagcgggattcccacccgatcatcaggcgtggcgtgagga
accgtggatccatcacgcccctcaggggggggaacagtagtcgcCATATGGAAGTCAGAAACCTTAATGCCGCTCGCGAGCT
GTTCCTGGACGAGCTCCTGGCCGCGTCGGCGGCGCCTGCCAGTGCCGAGCAGGAGG
AACTGCTGGCCCTGTTGCGCAGCGAGCGGATCGTGCTGGCCCACGCCGGCCAGCCG
CTGAGCGAGGCGCAAGTGCTCAAGGCGCTCGCCTGGTTGCTCGCGGCCAATCCGTC
CGCGCCTCCGGGGCAGGGCCTCGAGGTACTCCGCGAAGTCCTGCAGGCACGTCGGC
AGCCCGGTGCGCAGTGGGATCTGCGTGAGTTCCTGGTGTCGGCCTATTTCAGCCTGC
ACGGGCGTCTCGACGAGGATGTCATCGGTGTCTACAAGGATGTCCTGCAGACCCAG
GACGGCAAGCGCAAGGCGCTGCTCGACGAGCTCAAGGCGCTGACCGCGGAGTTGA
AGGTCTACAGCGTGATCCAGTCGCAGATCAACGCCGCGCTGTCGGCCAGGCAGGGC
ATCAGGATCGACGCTGGCGGTATCGATCTGGTCGACCCCACGCTATATGGCTATGCC
GTCGGCGATCCCAGGTGGAAGGACAGCCCCGAGTATGCGCTGCTGAGCAATCTGGA
TACCTTCAGCGGCAAGCTGTCGATCAAGGATTTTCTCAGCGGCTCGCCGAAGCAGA
GCGGGGAACTCAAGGGCCTCAGCGATGAGTACCCCTTCGAGAAGGACAACAACCCG
GTCGGCAATTTCGCCACCACGGTGAGCGACCGCTCGCGTCCGCTGAACGACAAGGT
CAACGAGAAGACCACCCTGCTCAACGACACCAGCTCCCGCTACAACTCGGCGGTCG
AGGCGCTCAACCGCTTCATCCAGAAATACGACAGCGTCCTGAGCGACATTCTCAGC
GCGATCGGATCCATGAACCCGATTACGCTGGAACGTGCTGGTCTGCCGTATGGTGTT
GCCGATGCTGGTGACATCCCGGCTCTGGGTCGCCCGGTCGCACGTGATGTGGAAAG
TCTGCGTGTTGAACGTCTGGCAGCACCGGCAGCTGCAAGCGCATCTGGCACCGGTG
TCGCTCTGACGCCGCCGTCTGCAGCAAGTCAGCAACGTCTGGAAGTTGCTAACCGC
GCGGAAATTGCCTCACTGGTCCAGGCAGTGGGTGAAGACGTGGGTCTGGCACGTCA
AGTGGTTCTGGCAGGTGCATCGACCCTGCTGAGCGCAGGTCTGATGTCGCCGCAGG
CGTTCGAAATTGAACTGGCCAAAATCACCGGCGAAGTTGAAAATCAGCAGAAAAAA
CTGAAACTGACGGAAATCGAACAGGCCCGTAAACAGAACCTGCAAAAAATGGAAG
ATAACCAGCAAAAAATCCGCGAATCGGAAGAAGCTGCGAAAGAAGCGCAGAAAAG
CGGCCTGGCCGCAAAAATTTTTGGTTGGATTTCTGCTATCGCGAGTATTATCGTGGG
TGCAATCATGGTTGCAACCGGTGTCGGTGCTGCAGCAGGTGCACTGATGATTGCTGG
CGGTGTCATGGGTGTCGTGAGTCAGTCCGTGCAGCAAGCAGCTGCGGATGGTCTGA
TCTCAAAAGAAGTGATGGAAAAACTGGGCCCGGCCCTGATGGGTATTGAAATGGCC
GTGGCACTGCTGGCCGCAGTTGTCTCCTTTGGTGGTTCAGCAGTTGGTGGTCTGGCA
CGTCTGGGTGCAAAAATCGGCGGTAAAGCTGCGGAAATGACGGCATCCCTGGCTTC
AAAAGTGGCAGACCTGGGCGGTAAATTCGGCTCTCTGGCGGGCCAGTCACTGTCGC
ATAGCCTGAAACTGGGTGTGCAAGTTTCTGATCTGACCCTGGACGTTGCAAACGGC
GCCGCACAGGCTACGCACAGTGGTTTTCAAGCGAAAGCTGCGAATCGTCAGGCCGA
TGTTCAAGAATCCCGTGCAGACCTGACCACGCTGCAGGGTGTCATTGAACGTCTGA
AAGAAGAACTGAGCCGCATGCTGGAAGCCTTTCAGGAAATTATGGAACGCATCTTC
GCAATGCTGCAAGCGAAAGGCGAAACCCTGCACAATCTGTCTTCCCGTCCGGCGGC
TATCTGAGGATCC
LTA1-PaF Amino acid sequence
SEQ ID NO: 8
MDNGDRLYRADSRPPDEIKRSGGLMPRGHNEYFDRGTQMNINLYDHARGTQTGFVRY
DDGYVSTSLSLRSAHLAGQSILSGYSTYYIYVIATAPNMENVNDVLGVYSPHPYEQEVS
ALGGIPYSQIYGWYRVNFGVIDERLHRNREYRDRYYRNLNIAPAEDGYRLAGFPPDHQ
AWREEPWIHHAPQGCGNSSRMEVRNLNAARELFLDELLAASAAPASAEQEELLALLRS
ERIVLAHAGQPLSEAQVLKALAWLLAANPSAPPGQGLEVLREVLQARRQPGAQWDLRE
FLVSAYFSLHGRLDEDVIGVYKDVLQTQDGKRKALLDELKALTAELKVYSVIQSQINAA
LSARQGIRIDAGGIDLVDPTLYGYAVGDPRWKDSPEYALLSNLDTFSGKLSIKDFLSGSP
KQSGELKGLSDEYPFEKDNNPVGNFATTVSDRSRPLNDKVNEKTTLLNDTSSRYNSAVE
ALNRFIQKYDSVLSDILSAIGSMNPITLERAGLPYGVADAGDIPALGRPVARDVESLRVE
RLAAPAAASASGTGVALTPPSAASQQRLEVANRAEIASLVQAVGEDVGLARQVVLAGA
STLLSAGLMSPQAFEIELAKITGEVENQQKKLKLTEIEQARKQNLQKMEDNQQKIRESEE
AAKEAQKSGLAAKIFGWISAIASIIVGAIMVATGVGAAAGALMIAGGVMGVVSQSVQQ
AAADGLISKEVMEKLGPALMGIEMAVALLAAVVSFGGSAVGGLARLGAKIGGKAAEM
TASLASKVADLGGKFGSLAGQSLSHSLKLGVQVSDLTLDVANGAAQATHSGFQAKAA
NRQADVQESRADLTTLQGVIERLKEELSRMLEAFQEIMERIFAMLQAKGETLHNLSSRP
AAI
SseB nucleic acid sequence
SEQ ID NO: 9
Atgtcttcaggaaacatcttatggggaagtcaaaaccctattgtgtttaaaaatagcttcggcgtcagcaacgctgataccgggagccag
gatgacttatcccagcaaaatccgtttgccgaagggtatggtgttttgcttattctccttatggttattcaggctatcgcaaataataaa
tttattgaagtccagaagaacgctgaacgtgccagaaatacccaggaaaagtcaaatgagatggatgaggtgattgctaaagcagccaaa
ggggatgctaaaaccaaagaggaggtgcctgaggatgtaattaaatacatgcgtgataatggtattctcatcgatggtatgaccattgat
gattatatggctaaatatggcgatcatgggaagctggataaaggtggcctacaggcgatcaaagcggctttggataatgacgccaaccgg
aataccgatcttatgagtcaggggcagataacaattcaaaaaatgtctcaggagcttaacgctgtccttacccaactgacagggcttatc
agtaagtggggggaaatttccagtatgatagcgcagaaaacgtactca
SscB amino acid sequence
SEQ ID NO: 10
MSSGNILWGSQNPIVFKNSFGVSNADTGSQDDLSQQNPFAEGYGVLLILLMVIQAIANN
KFIEVQKNAERARNTQEKSNEMDEVIAKAAKGDAKTKEEVPEDVIKYMRDNGILIDGM
TIDDYMAKYGDHGKLDKGGLQAIKAALDNDANRNTDLMSQGQITIQKMSQELNAVLT
QLTGLISKWGEISSMIAQKTYS
LTA1-SscB Nucleic acid sequence
SEQ ID NO: 11
ATGGGCAGCAGCCATCATCATCATCATCACAGCAGCGGCCTGGTGCCGCGCGGCAG
CCATatggacaatggcgatcgtttataccgtgccgactcgcgtcccccagatgagattaaacgtagcggtgggttaatgccacgtgggc
acaatgagtattttgaccgtggaacacagatgaacattaacctttacgatcatgcccgtgggacccagaccgggtttgtccgttatgatg
acgggtatgttagtacgagtttgtccttacgctccgcacaccttgcgggacaaagtattttatcaggctacagcacatattacatttatg
tgatcgccactgccccaaacatgttcaatgtgaacgatgtgttgggggtttacagcccccatccatatgaacaagaagtctcggcccttg
gggggatcccatatagccagatttatggttggtaccgcgtaaattttggtgtgattgatgaacgtttgcatcgtaaccgtgaataccgcg
atcgctactaccgtaacttgaacattgcacctgccgaggacggctatcgtttagcgggattcccacccgatcatcaggcgtggcgtgagg
aaccgtggatccatcacgcccctcaggggtgcgggaacagtagtcgcgggtccgcggcatccatgtcttcaggaaacatcttatggggaa
gtcaaaaccctattgtgtttaaaaatagcttcggcgtcagcaacgctgataccgggagccaggatgacttatcccagcaaaatccgtttg
ccgaagggtatggtgttttgcttattctccttatggttattcaggctatcgcaaataataaatttattgaagtccagaagaacgctgaac
gtgccagaaatacccaggaaaagtcaaatgagatggatgaggtgattgctaaagcagccaaaggggatgctaaaaccaaagaggaggtgc
ctgaggatgtaattaaatacatgcgtgataatggtattctcatcgatggtatgaccattgatgattatatggctaaatatggcgatcatg
ggaagctggataaaggtggcctacaggcgatcaaagcggctttggataatgacgccaaccggaataccgatcttatgagtcaggggcaga
taacaattcaaaaaatgtctcaggagcttaacgctgtccttacccaactgacagggcttatcagtaagtggggggaaatttccagtatga
tagcgcagaaaacgtactcataaGGATCC
LTA1-SseB Amino acid sequence
SEQ ID NO: 12
MGSSHHHHHHSSGLVPRGSHMDNGDRLYRADSRPPDEIKRSGGLMPRGHNEYFDRGT
QMNINLYDHARGTQTGFVRYDDGYVSTSLSLRSAHLAGQSILSGYSTYYIYVIATAPNM
FNVNDVLGVYSPHPYEQEVSALGGIPYSQIYGWYRVNFGVIDERLHRNREYRDRYYRN
LNIAPAEDGYRLAGFPPDHQAWREEPWIHHAPQGCGNSSRGSAASMSSGNILWGSQNPI
VFKNSFGVSNADTGSQDDLSQQNPFAEGYGVLLILLMVIQAIANNKFIEVQKNAERARN
TQEKSNEMDEVIAKAAKGDAKTKEEVPEDVIKYMRDNGILIDGMTIDDYMAKYGDHG
KLDKGGLQAIKAALDNDANRNTDLMSQGQITIQKMSQELNAVLTQLTGLISKWGEISS
MIAQKTYS*
LTA1 nucleic acid sequence
SEQ ID NO: 13
CATAtggacaatggcgatcgtttataccgtgccgactcgcgtcccccagatgagattaaacgtagcggtgggttaatgccacgtgggca
caatgagtattttgaccgtggaacacagatgaacattaacctttacgatcatgcccgtgggacccagaccgggtttgtccgttatgatga
cgggtatgttagtacgagtttgtccttacgctccgcacaccttgcgggacaaagtattttatcaggctacagcacatattacatttatgt
gatcgccactgccccaaacatgttcaatgtgaacgatgtgttgggggtttacagcccccatccatatgaacaagaagtctcggcccttgg
ggggatcccatatagccagatttatggttggtaccgcgtaaattttggtgtgattgatgaacgtttgcatcgtaaccgtgaataccgcga
tcgctactaccgtaacttgaacattgcacctgccgaggacggctatcgtttaggggattcccacccgatcatcaggcgtggcgtgaggaa
ccgtggatccatcacgcccctcaggggtgcgggaacagtagtcgc
LTA1 amino acid sequence
SEQ ID NO: 14
MDNGDRLYRADSRPPDEIKRSGGLMPRGHNEYFDRGTQMNINLYDHARGTQTGFVRY
DDGYVSTSLSLRSAHLAGQSILSGYSTYYIYVIATAPNMFNVNDVLGVYSPHPYEQEVS
ALGGIPYSQIYGWYRVNFGVIDERLHRNREYRDRYYRNLNIAPAEDGYRLAGFPPDHQ
AWREEPWIHHAPQGCGNSSR
22BF Amino Acid Sequence
SEQ ID NO: 15
MTIDLGVSLTSQAGGLGIDLKSMDIQTLMVYVQGRRAELLTAQMQTQAEVVQKANE
RMAQLNEVLSALSRAKAEFPPNPKPGDTIPGWDNQKVSRIEVPLNDALRAAGLTGMFE
ARDGQVTAPGGRGTQVVNGTGVMAGSTTYKELESAYTTVKGMLDTASNTQQMDMIR
LQAASNKRNEAFEVMTNTEKRRSDLNSSITNNMRKLMTVMSTTISTAPSGAALAPSRID
MRAPEPGSAGEGAGILAPVTTLALAAGRPAFPASPSLRTAPVLDPPVRDLSPADLADLLR
VLRSRAVDGQLATARENLQDAQVKAKQNTQAQLDKLDAWFRKAEEAESKGWLSKVF
GWIGKVLAVVASALAVGFAAVASVATGAAATPMLLLSGMALVSAVTSLADQISQEAG
GPPISLGGFLSGLAGRLLTALGVDQSQADQIAKIVAGLA VPVVLLIEPQMLGEMAQGVA
RLAGASDATAGYIAMAMSIVAAIAVAAINAAGTAGAGSASAIKGAWDRAAAVATQVL
QGGTAVAQGGVGVSMAVDRKQADLLVADKADLAASLTKLRAAMEREADDIKKILAQ
FDEAYHMIAKMISDMASTHSQVSANLGRRQAV
Bsp22 Nucleotide Sequence
SEQ ID NO: 16
CATatgaccattgatctcggagtttcactcacgtcgcaggccggcggcctgcaaggcatcgacctcaagagcatggatatccagactct
catggtgtatgtgcagggtcgtcgcgccgaactcctcacggctcaaatgcagacccaggccgaagtggtgcagaaggccaatgaacgca
tggcgcagctcaacgaggtcctgtccgcgctgtcccgggccaaggccgagtttccgcccaatccgaagccgggcgacaccatcccggg
ctgggacaaccagaaggtcagccggatcgaggttcctctcaatgatgcgctgcgcgctgccggcctgacgggcatgttcgaagcgcgcg
atggccaagtgaccgcccccggcggccggggtacgcaggtcgtgaacggcacgggcgtcatggccggttccacgacctataaggaact
cgaaagtgcctacaccaccgtaaaggggatgctggatacggcgtccaatacgcaacagatggacatgatcaggctgcaggccgccagc
aacaagcgcaacgaggctttcgaggtcatgaccaacaccgagaagcggcgcagcgacctgaacagttccatcaccaacaacatgcgc
Bsp22 Amino Acid Sequence
SEQ ID NO: 17
MTIDLGVSLTSQAGGLQGIDLKSMDIQTLMVYVQGRRAELLTAQMQTQAEVVQKANE
RMAQLNEVLSALSRAKAEFPPNPKPGDTIPGWDNQKVSRIEVPLNDALRAAGLTGMFE
ARDGQVTAPGGRGTQVVNGTGVMAGSTTYKELESAYTTVKGMLDTASNTQQMDMIR
LQAASNKRNEAFEVMTNTEKRRSDLNSSITNNMR
BopB Nucleotide Sequence
SEQ ID NO: 18
Atgaccgtcatgagtacgaccatatccacagccccgagcggcgccgcgcttgcgccgtctcgcatagatatgcgggcaccggagcccg
ggagtgccggcgaaggcgccggcatcctggcgccggtgacgacgctggctctggcggcgggccggccggcttttccagcgtcaccgt
cgctgcgcaccgcgcccgtcctggatccgccagtgcgcgatctcagccccgccgacttggccgacctgctgcgcgtcttgcgatccagg
gcggtggacgggcagttggccacggcgcgcgagaacctgcaggacgcgcaagtcaaggcgaagcagaacacccaggcccagctcg
acaagctggacgcatggtttcggaaggccgaagaggccgagagcaagggatggctgagcaaggtgttcggctggatcggcaaggtgct
ggcggtcgtggcatcggccctggcggtgggctttgccgccgtcgccagcgtggccaccggcgcggcggccacacccatgctgctgctc
agcggcatggcactggtcagcgccgtgacatcgctggccgaccagatatcgcaagaggcgggaggcccgcctatcagcctgggcggg
tttctctccgggctggccggacgtctgctgacagcgttgggggtggatcagtcgcaggccgaccaaattgccaagatcgtcgccggcctg
gccgtgcccgtcgtcttgctgatcgaaccccagatgctgggcgaaatggcgcaaggcgtggccaggctggctggcgccagcgatgcca
ccgggggtacatagccatggcgatgtccatcgtggcggcgatcgcggtcgccgcgatcaatgccgccggtacagccggcgcgggtag
cgcttcggcgatcaagggggcctgggatcgggccgccgcggtagccacccaggtccttcaagggggtacggcagtggcgcaaggcg
gcgtcggcgtgtcgatggcagtcgatcgcaaacaggccgatctcctggtcgccgacaaggcggatctggcggcgagcctgacaaaact
gcgggcggccatggagcgtgaggcggacgatatcaagaagatcctggctcaattcgacgaggcctatcacatgatcgcgaagatgatca
gcgatatggcgagtacgcacagccaggtcagcgccaacctcgggggcgccaggcggtgtagCTCGAG
BopB Amino Acid Sequence
SEQ ID NO: 19
MTVMSTTISTAPSGAALAPSRIDMRAPEPGSAGEGAGILAPVTTLALAAGRPAFPASPSL
RTAPVLDPPVRDLSPADLADLLRVLRSRAVDGQLATARENLQDAQVKAKQNTQAQLD
KLDAWFRKAEEAESKGWLSKVFGWIGKVLAVVASALAVGFAAVASVATGAAATPML
LLSGMALVSAVTSLADQISQEAGGPPISLGGFLSGLAGRLLTALGVDQSQADQIAKIVAG
LAVPVVLLIEPQMLGEMAQGVARLAGASDATAGYIAMAMSIVAAIAVAAINAAGTAG
AGSASAIKGAWDRAAAVATQVLQGGTAVAQGGVGVSMAVDRKQADLLVADKADLA
ASLTKLRAAMEREADDIKKILAQFDEAYHMIAKMISDMASTHSQVSANLGRRQAV
LTA1-GSAAS-IpaD-LE-IpaB (DBF) Amino Acid sequence
SEQ ID NO: 20
MDNGDRLYRADSRPPDEIKRSGGLMPRGHNEYFDRGTQMNINLYDHARGTQTGFVRY
DDGYVSTSLSLRSAHLAGQSILSGYSTYYIYVIATAPNMFNVNDVLGVYSPHPYEQEVS
ALGGIPYSQIYGWYRVNFGVIDERLHRNREYRDRYYRNLNIAPAEDGYRLAGFPPDHQ
AWREEPWIHHAPQGCGNSSRGSAASMNITTLTNSISTSSFSPNNINGSSTETVNSDIKTTT
SSHPSSLTMLNDTLHNIRTTNQALKKELSQKTLRNEYPINKDARELLHSAPKEAELDGD
QMISHRELWAKIANSINDINEQYLKVYEHAVSSYTQMYQDFSAVLSSLAGWISPGGNDG
NSVKLQVNSLKKALEELKEKYKDKPLYPANNTVSQEQANKWLTELGGTIGKVSQKNG
GYVVSINMTPIDNMLKSLDNLGGNGEVVLDNAKYQAWNGFSAEDETMKNNLQTLVQ
KYSNANSIFDNLVKVLSSTISSCTDTDKLFLHFLEMHNVSTTTTGFPLAKILTSTELGDNT
IQAANDAANKLFSLTIADLTANQNINTTNAHSTSNILIPELKAPKSLNASSQLTLLIGNLIQ
ILGEKSLTALTNKITAWKSQQQARQQKNLEFSDKINTLLSETEGLTRDYEKQINKLKNA
DSKIKDLENKINQIQTRLSNLDPESPEKKKLSREEIQLTIKKDAAVKDRTLIEQKTLSIHSK
LTDKSMQLEKEIDSFSAFSNTASAEQLSTQQKSLTGLASVTQLMATFIQLVGKNNEESLK
NDLALFQSLQESRKTEMERKSDEYAAEVRKAEELNRVMGCVGKILGALLTIVSVVAAA
FSGGASLALAAVGLALMVTDAIVQAATGNSFMEQALNPIMKAVIEPLIKLLSDAFTKML
EGLGVDSKKAKMIGSILGAIAGALVLVAAVVLVATVGKQAAAKLAENIGKIIGKTLTDL
IPKFLKNFSSQLDDLITNAVARLNKFLGAAGDEVISKQIISTHLNQAVLLGESVNSATQA
GGSVASAVFQNSASTNLADLTLSKYQVEQLSKYISEAIEKFGQLQEVIADLLASMSNSQ
ANRTDVAKAILQQTTA

What is claimed is:

1. A fusion polypeptide comprising i) a fusion of a needle tip protein or an antigenic fragment thereof and/or a translocator protein or an antigenic fragment thereof from a Type III secretion system (T3SS) of a Gram negative bacteria and ii) a double mutant labile toxin (dmLT) or an antigenic fragment thereof from Enterotoxigenic Escherichia coli or cholera toxin or an antigenic fragment thereof.

2. The polypeptide of claim 1, wherein the fusion polypeptide is arranged so that the needle tip protein is 5′ of the translocator protein.

3. The polypeptide of claim 1, wherein the gram negative bacteria comprises Pseudomonas spp., Shigella spp, Salmonella enterica, Bordetella spp., Burkholderia spp., or Yersinia spp.

4. The polypeptide of claim 1, wherein the needle tip protein comprises PcrV, IpaD, SseB, Bsp22, LcrV, or BipD.

5. The polypeptide of claim 1, wherein the translocator protein comprises PopB, IpaB, SseC, BopB, YopB, or BipB.

6. The polypeptide of claim 1, further comprising Pseudomonas spp exolysin A (ExlA).

7. The polypeptide of any of claim 1, wherein the LTA1 is 5′ of the needle tip protein and/or translocator protein fusion.

8. The polypeptide of claim 1, wherein the fusion comprises the sequence as set forth in SEQ ID NO: 41.

9. The polypeptide of claim 1, wherein the dmLT comprises the active moiety LTA1.

10. The polypeptide of claim 1, wherein the dmLT retains its ADP ribosylation activity.

11. The polypeptide of claim 1, wherein the dmLT is 5′ of the fusion of IpaD and IpaB.

12. The polypeptide of claim 1, wherein polypeptide comprises the amino acid sequence as set forth in SEQ ID NO: 16, SEQ ID NO: 28, or SEQ ID NO: 38.

13. A vaccine comprising one or more of the fusion polypeptides of claim 1.

14. The vaccine of claim 8, further comprising MedImmune Emulsion (ME), Chitosan-C48/80 (Chi) nanoparticles, Bacterial Enzymatic Combinatorial Chemistry (BECC) candidate 438 (BECC438), and/or BECC470.

15. A method of treating, inhibiting, reducing, ameliorating, and/or preventing an infection of a Gram negative bacterial infection in a subject comprising administering to the subject the fusion polypeptide of claim 1.

16. The method of claim 15, wherein the bacteria comprises Pseudomonas spp., Shigella spp, Salmonella enterica, Bordetella spp., or Burkholderia spp.

17. A method of eliciting an immune response in a subject to a Gram negative bacteria comprising administering to the subject one or more of the fusion polypeptide of claim 1.

18. The method of claim 17, wherein the bacteria comprises Pseudomonas spp., Shigella spp, Salmonella enterica, Bordetella spp., or Burkholderia spp.
